(19) United States
(12) Patent Application Publication
(10) Pub. No.: US 2007/0123492 A1
(43) Pub. Date: May 31, 2007

(54) Analogs of lysophosphatidic acid and methods of making and using thereof

(76) Inventors: Glenn D. Prestwich, Salt Lake City, UT (US); Yong Xu, Salt Lake City, UT (US); Lian Qian, King of Prussia, PA (US)

Correspondence Address: NEEDLE & ROSENBERG, P.C., SUITE 1000, 999 PEACHTREE STREET, ATLANTA, GA, 30309-3915, US

(21) Appl. No.: 10/552,382

(22) PCT Filed: Apr. 9, 2004
(86) PCT No.: PCT/US04/11060

§ 371 Date: Aug. 28, 2006

Related U.S. Application Data
(60) Provisional application No. 60/462,095, filed on Apr. 9, 2003.

(51) Int. Cl. A61K 31/675; C07F 9/06; A61K 31/66

(52) U.S. Cl. 514/79; 514/109; 546/22; 548/413; 558/83; 544/243

(57) ABSTRACT

Described herein are analogs of lysophosphatidic acid. Also described herein are methods of making and using analogs of lysophosphatidic acid.

### CROSS REFERENCE TO RELATED APPLICATIONS

This application is a continuation-in-part of U.S. provisional application Ser. No. 60/462,095, filed Apr. 9, 2003. This application is hereby incorporated by this reference in its entirety for all of its teachings.

### ACKNOWLEDGEMENTS

The research leading to this invention was funded in part by the National Institutes of Health, Grant No. NS 29632. The U.S. Government may have certain rights in this invention.

### BACKGROUND

Lysophosphatidic acid (1- or 2-O-acyl-sn-glycero-3-phosphate, sn-1 or sn-2 LPA), a simple phospholipid, is an intercellular signaling molecule with a variety of biologic effects¹. LPA induces cell proliferation, morphological changes, and has been shown to be involved in many physiological and pathological processes including neurogenesis², myelination, angiogenesis³, wound healing⁴, and cancer progression⁵.

Normally, LPA is present in serum at low levels and is not detectable in platelet-poor plasma, whole blood, or cerebrospinal fluid. LPA is present at elevated levels, however, in the ascites of ovarian cancer patients and may thus contribute to the progression of human cancer⁶. Interestingly, LPA produced by stimulated platelets is chemically distinct from that found in ascites of ovarian cancer patients. sn-1 LPA is preferentially produced in platelets, whereas sn-2 type is found to be predominant in ascites. Therefore, levels of sn-2 LPA seem to be associated with the initiation and progression of ovarian cancer⁷. On the other hand, it has been demonstrated that sn-2 LPA is not stable under physiological conditions; it is rapidly converted to sn-1 LPA and vis versa as a result of intramolecular acyl chain migration. This reaction, facilitated by acidic and basic conditions, yields an equilibrium mixture of 1-acyl and 2-acyl-sn-glycerol-3-phosphate favoring the 1-acyl isomer. The instability of 2-acyl-sn-glycerol-3-phosphate is therefore a challenge against isolation and structure-activity studies of individual LPA species.

Although three mammalian genes, Edg-2/LPA₁, Edg-4/LPA₂, and Edg-7/LPA₃ encoding high-affinity LPA receptors have been cloned and characterized⁸, the function of particular receptors in the mammalian system and the molecular mechanism of LPA actions have not been elucidated⁹. Among the reasons for this ignorance is the lack of molecular tools, especially the metabolically stable and selective ligands for LPA receptors¹⁰. Described herein are LPA analogs with improved stability and/or with receptor-selective activity. One approach is to produce and investigate acylic analogs of LPA. Another approach involves the preparation and analysis of cyclic analogs of LPA. Although cyclic compounds are known¹¹⁻¹⁹, the cyclic as well as acyclic analogs described herein possess improved metabolic stability and biological activity.

### SUMMARY

Described herein are analogs of lysophosphatidic acid. Also described herein are methods of malking and using analogs of lysophosphatidic acid.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 shows a reaction scheme for producing a diol having the formula III.

FIG. 2 shows a reaction scheme for converting a diol having the formula III to other derivatives.

FIG. 3 shows a reaction scheme for producing α,α-difluoro compounds described herein.

FIG. 4 shows a reaction scheme for producing α,α-difluoro compounds described herein.

FIG. 5 shows a reaction scheme for producing difluoro compounds described herein.

FIG. 6 shows a reaction scheme for producing hydroxyethoxy compounds described herein.

FIG. 7 shows a reaction scheme for producing hydroxyethoxy compounds described herein.

FIG. 8 shows a reaction scheme for producing α-monofluoro compounds described herein.

FIG. 9 shows a reaction scheme for producing α-monofluoro compounds described herein.

FIG. 10 shows a reaction scheme for producing α-monofluoro compounds described herein.

FIG. 11 shows a reaction scheme for producing α-monofluoro compounds described herein.

FIG. 12 shows a reaction scheme for producing α-monofluoro compounds described herein.

FIG. 13 shows a reaction scheme for producing α-monofluoro compounds described herein.

FIG. 14 shows a reaction scheme for producing α-monofluoro compounds described herein.

FIG. 15 shows a reaction scheme for producing α-monofluoro compounds described herein.

FIG. 16 shows the structures of selected known analogs of LPA described herein.

FIG. 17 shows a reaction scheme for producing cyclic analogs of LPA described herein.

FIG. 18 shows a proposed reaction scheme for producing cyclic analogs of LPA described herein.

FIG. 19 shows a proposed reaction scheme for producing cyclic analogs of LPA described herein.

FIG. 20 shows a proposed reaction scheme for producing cyclic analogs of LPA described herein.

FIG. 21 shows a reaction scheme for producing cyclic analogs of LPA described herein.

FIG. 22 shows a reaction scheme for producing cyclic analogs of LPA described herein.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Variables such as R¹, R², R³, R⁶, R⁷, X¹, X², Y¹, Y², U, V, W, and Z used throughout the application are the same variables as previously defined unless stated to the contrary.

The term “substantially” with respect to the stereochemistry at carbon a refers to greater than 95%, greater than 97%, greater than 98%, greater than 99%, greater than 99.5%, or 100% of one enantiomer with respect to the other enantiomer. The terms “R” and “S” with respect to the stereochemistry at carbon a are also referred to in the art as “D” and “L,” respectively.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Examples of longer chain alkyl groups include, but are not limited to, an oleate group or a pahnitate group. A “lower alkyl” group is an alkyl group containing from one to six carbon atoms.

The term “cycloalkyl group” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “silyl group” as used herein is represented by the formula —SiRR′R″, where R, R′, and R″ can be, independently, hydrogen, an alkyl, aryl, cycloalkyl, halogenated alkyl, alkoxy, or heterocycloalkyl group described above.

The term “protecting group” as used herein is a group that can be chemically bound to an oxygen atom, and subsequently removed (either chemically, in-vitro, or in-vivo) from the oxygen atom by predictable methods. Examples of many of the possible protective groups can be found in Protective Groups in Organic Synthesis by T. W. Green, John Wiley and Sons, 1981, which is incorporated herein by reference in its entirety.

The term “cationic counterion” as used herein is any ion bearing a positive charge. The cationic counterion can be mono- or multivalent.

I. Analogs of LPA

a. Acylic Compounds

In one aspect described herein is a compound having the formula I 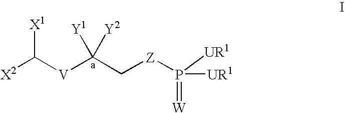
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur,
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
   or the pharmaceutically acceptable salt or ester thereof,
   wherein when Y¹ and Y² are different groups, the stereochemistry at carbon a is either substantially R or substantially S, and
   wherein the compound having the formula I is not 1-acyl-sn-glycerol 3-phosphate and 2-acyl-sn-glycerol 3-phosphate.
   The compounds 1-acyl-sn-glycerol 3-phosphate and 2-acyl-sn-glycerol 3-phosphate are generally referred to as lysophosphatidic acid (LPA).

In one aspect, both of R¹ can be part of a cyclo or heterocyclo group. For example, the heterocyclic group can be morpholino, piperidino, etc.

In one aspect, compounds having the formula I are monofluoro compounds. In one aspect, each U comprises oxygen, W is oxygen, V is not present, X¹ is hydrogen, and X² is fluorine. In another aspect, each U comprises oxygen, W is oxygen, V is not present, Z is oxygen, X¹ is hydrogen, and X² is fluorine. In another aspect, each U comprises oxygen, W is oxygen, V is not present, Z is oxygen, X¹ is hydrogen, X² is fluorine, Y¹ is hydrogen, and Y² is OC(O)R³, wherein R³ is a branched or straight chain C₁ to C₂₅ alkyl group, and R¹ is hydrogen. In another aspect, Z is oxygen, X¹ is hydrogen, X² is fluorine, Y¹ is hydrogen, and Y² is OC(O)R³, wherein R³ is an oleate group or a palmitate group, and R¹ is hydrogen, and the stereochemistry at carbon a is R or S.

In another aspect, the monofluoro compound is a compound having the formula I, wherein each U comprises oxygen, W is oxygen, V is not present, Z is oxygen, Y¹ is hydrogen, and Y² is fluorine. In another aspect, each U comprises oxygen, W is oxygen, V is not present, Z is oxygen, Y¹ is hydrogen, Y² is fluorine, X¹ is hydrogen, X² is OC(O)R³, wherein R³ is a branched or straight chain C₁ to C₂₅ alkyl group, and each R¹ is hydrogen. In a further aspect, each U comprises oxygen, W is oxygen, V is not present, Z is oxygen, Y¹ is hydrogen, Y² is fluorine, X¹ is hydrogen, X² is OC(O)R³, wherein R³ is an oleate group or a palmitate group, wherein the stereochemistry at carbon a is R or S.

In another aspect, the monofluoro compound is a compound having the formula I, wherein each U comprises oxygen, W is oxygen, V is not present, Z is CBF, Y¹ is hydrogen, Y² is a hydroxyl group. In one aspect, each U comprises oxygen, W is oxygen, V is not present, Z is CHF, Y¹ is hydrogen, Y² is a hydroxyl group, X¹ is hydrogen, X² is OC(O)R³, wherein R³ is a branched or straight chain C₁ to C₂₅ alkyl group, and each R¹ is hydrogen. In one aspect, each U comprises oxygen, W is oxygen, V is not present, Z is CHF, Y¹ is hydrogen, Y² is a hydroxyl group, X¹ is hydrogen, X² is OC(O)R³, wherein R³ is an oleate group or a palmitate group, and each R¹ is hydrogen, wherein the stereochemistry at carbon a is R or S.

In another aspect, each U comprises oxygen, W is oxygen, V is not present, Z is CHF, Y¹ is hydrogen, and Y² is a hydroxyl group. In one aspect, each U comprises oxygen, W is oxygen, V is not present, X¹ is hydrogen, X² is OC(O)R³, wherein R³ is a branched or straight chain C₁ to C₂₅ alkyl group, and each R¹ is ethyl. In a further aspect, each U comprises oxygen, W is oxygen, V is not present, Z is CHF, Y¹ is hydrogen, Y² is a hydroxyl group, X¹ is hydrogen, X² is a silyl group or an alkyl group, and each R¹ is ethyl.

In another aspect, each U comprises oxygen, W is oxygen, V is not present, Z is CHF, Y¹ is hydrogen, and Y² is an alkyl group. In one aspect, each U comprises oxygen, W is oxygen, V is not present, Z is CHF, Y¹ is hydrogen, Y² is a hydroxyl group, X¹ is hydrogen, X² is a silyl group, a hydroxyl group, or OC(O)R³, wherein R³ is a branched or straight chain C₁ to C₂₅ alkyl group, and each R¹ is ethyl or each R¹ is hydrogen.

In a further aspect, each U comprises oxygen, W is oxygen, V is not present, Z is CHF, Y¹ is hydrogen, and Y² is a hydroxyl group. In another aspect, each U comprises oxygen, W is oxygen, V is not present, Z is CHF, Y¹ is hydrogen, Y² is a hydroxyl group, X¹ is hydrogen, X² is an alkyl group, and each R¹ is ethyl or each R¹ is hydrogen.

Methods for preparing monofluoro compounds having the formula I are presented below.

In another aspect, the compound having the formula I is a difluoro compound, wherein Z is CF₂. In one aspect, each U comprises oxygen, W is oxygen, V is not present, Z is CF₂, Y¹ is hydrogen, Y² is OC(O)R³, wherein R³ is a branched or straight chain C₁ to C₂₅ alkyl group, and each R¹ is an ethyl group or a sodium ion. In one aspect, each U comprises oxygen, W is oxygen, V is not present, Z is CF₂, Y¹ is hydrogen, Y² is OC(O)R³, wherein R³ is a branched or straight chain C₁ to C₂₅ alkyl group, each R¹ is an ethyl group or a sodium ion, X¹ is hydrogen and X² is OH or OC(O)R³, wherein R³ is a branched or straight chain C₁ to C₂₅ alkyl group, wherein the stereochemistry at carbon a is R or S.

In another aspect, each U comprises oxygen, W is oxygen, V is not present, Z is CF₂, X¹ is hydrogen, X² is OC(O)R³, wherein R³ is a branched,or straight chain C₁ to C₂₅ alkyl group, and each R¹ is an ethyl group or a sodium ion. In a further aspect, each U comprises oxygen, W is oxygen, V is not present, Z is CF₂, X¹ is hydrogen, X² is OC(O)R³, wherein R³ is a branched or straight chain C₁ to C₂₅ alxyl group, each R¹ is an ethyl group or a sodium ion, Y¹ is hydrogen and Y² is OH or OC(O)R³, wherein R³ is a branched or straight chain C₁ to C₂₅ aLkyl group, wherein the stereochemistry at carbon a is R or S.

In another aspect, each U comprises oxygen, W is oxygen, V is not present, Z is CF₂, X¹ is hydrogen, X² is OH, Y¹ is hydrogen, Y² is OH, and each R¹ is an ethyl group.

Methods for preparing difluoro compounds having the formula I where Z is CF₂ are described below in the Examples section.

In another aspect, the compounds having the formula I are difluoro compounds, wherein each U comprises oxygen, W is oxygen, V is not present, Z is CH₂ and X¹ and X² are fluorine. In one aspect, each U comprises oxygen, W is oxygen, V is not present, Z is CH₂, X¹ and X² are fluorine, Y¹ is hydrogen, Y² is a hydroxyl group, OR², or OC(O)R³. In another aspect, each U comprises oxygen, W is oxygen, V is not present, Z is CH₂, X¹ and X² are fluorine, Y¹ is hydrogen, Y² is a hydroxyl group, OR², or OC(O)R³, and each R¹ is hydrogen or a methyl group, wherein the stereochemistry at carbon a is R or S.

Methods for preparing difluoro compounds having the formula I where Z is CH₂ and X¹ and X² are fluorine are described in the Examples section below.

In another aspect, the compounds having the formula I are nonfluoro compounds. In one aspect, each U comprises oxygen, W is oxygen, V is not present, Z is oxygen, Y¹ is hydrogen, and Y² is OCH₂CH₂OR², wherein R² is hydrogen or a protecting group. In another aspect, each U comprises oxygen, W is oxygen, V is not present, Z is oxygen, Y¹ is hydrogen, Y² is OCH₂CH₂OR², wherein R² is hydrogen or a protecting group, X¹ is hydrogen, and X² is OC(O)R³, wherein R³ is a branched or straight chain C₁ to C₂₅ alkyl group. In a further aspect, each U comprises oxygen, W is oxygen, V is not present, Z is oxygen, Y¹ is hydrogen, Y² is OCH₂CH₂OR², wherein R² is hydrogen or a protecting group, X¹ is hydrogen, and X² is OC(O)R³, wherein R³ is a branched or straight chain C₁ to C₂₅ alkyl group, each R¹ is a methyl group or hydrogen, and the stereochemistry at carbon a is R or S.

In another aspect, the compounds having the formula I are nonfluoro compounds, wherein each U comprises oxygen, W is oxygen, V is not present, Z is oxygen, X¹ is hydrogen and X² is OCH₂CH₂OR², wherein R² is hydrogen or a protecting group. In one aspect, each U comprises oxygen, each U comprises oxygen, W is oxygen, V is not present, Z is oxygen, X¹ is hydrogen, X² is OCH₂CH₂OR², wherein R² is hydrogen or a protecting group, Y¹ is hydrogen, and Y² is OC(O)R³, wherein R³ is a branched or straight chain C₁ to C₂₅ alkyl group. In a further aspect, each U comprises oxygen, W is oxygen, V is not present, Z is oxygen, X¹ is hydrogen, X² is OCH₂CH₂OR², wherein R² is hydrogen or a protecting group, Y¹ is hydrogen, and Y² is OC(O)R³, wherein R³ is a branched or straight chain C₁ to C₂₅ alkyl group, each R¹ is a methyl group or hydrogen, and the stereochemistry at carbon a is R or S.

Methods for preparing nonfluoro compounds having the formula I discussed above are described below in the Examples section.

In one aspect, when V is not present in formula I, each U comprises oxygen, W is oxygen, X¹ and Y¹ are hydrogen, and X² is hydroxyl, then Y² is not hydroxyl.

In one embodiment, the compounds having the formula I are presented in Table 1 below.

TABLE 1

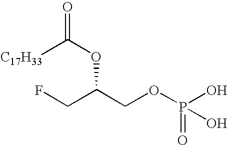

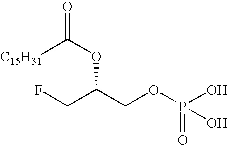

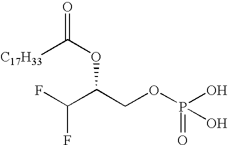

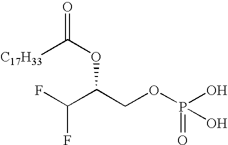

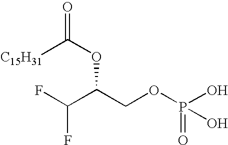

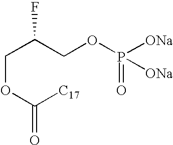

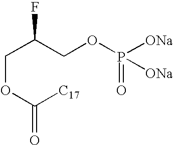

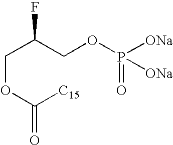

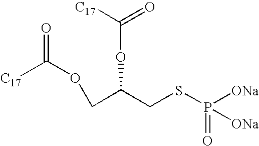

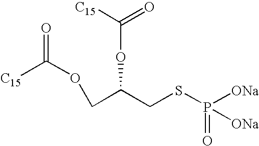

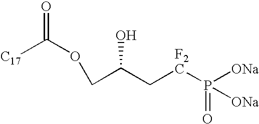

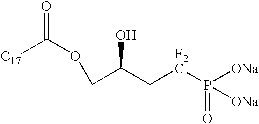

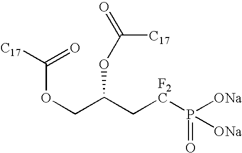

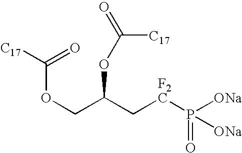

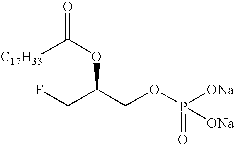

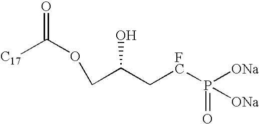

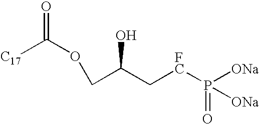

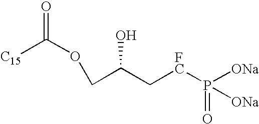

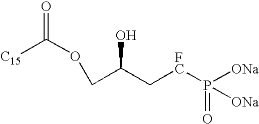

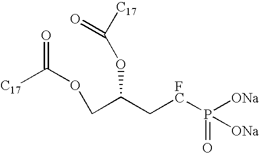

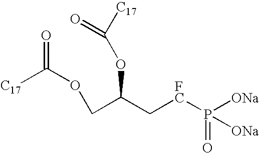

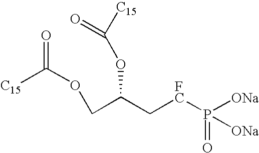

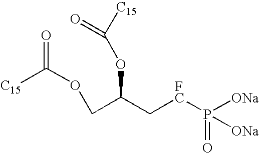

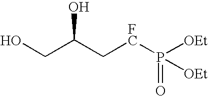

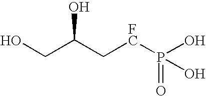

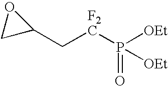

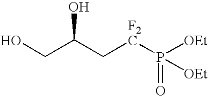

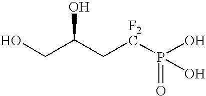

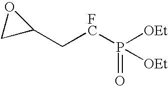

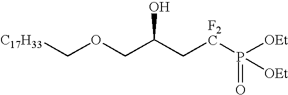

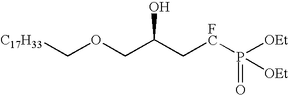

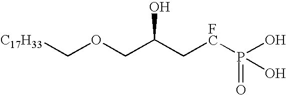

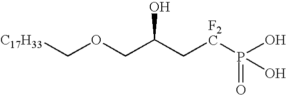

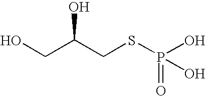

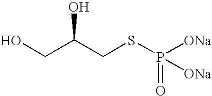

b. Cyclic Compounds

In one embodiment described herein are compounds having the formula VII 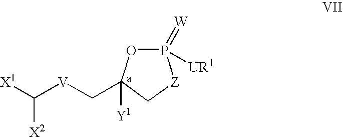
wherein
X¹, X², and Y¹ comprises, independently, hydrogen, fluorine, a hydroxyl group, a branched or straight chain C₁ to C₂₅ alkyl group, OR², OCH₂CH₂OR², OC(O)R³, or NC(O)R³;
U comprises oxygen, sulfur, or NR¹;
V is not present or when V is present, V comprises oxygen or sulfur;
W comprises oxygen or sulfur,
Z comprises oxygen, sulfur, NR¹, CH₂, CHF, CF₂, or CHOR²;
each R¹ comprises hydrogen, a branched or straight chain C₁ to C₂₅ alkyl group, or a cationic counterion;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group;
   or the pharmaceutically acceptable salt or ester thereof,
   wherein the stereochemistry at carbon a is either substantially R or substantially S,
   wherein when W is oxygen, V is not present, X¹ and Y¹ are hydrogen, and X² is OC(O)R³, then Z is not CH₂ or oxygen.

In another aspect, described herein are compounds having the formula VII 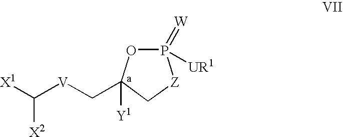
wherein
X¹, X², and Y¹ comprises, independently, hydrogen, fluorine, a hydroxyl group, a branched or straight chain C₁ to C₂₅ alkyl group, OR², OCH₂CH₂OR², OC(O)R³, or NC(O)R³;
U comprises oxygen, sulfur, or NR¹;
V is not present or when V is present, V comprises oxygen or sulfur;
W comprises oxygen or sulfur;
Z comprises sulfur, NR¹, CHF, CF₂, or CHOR²;
Each R¹ comprises hydrogen, a branched or straight chain C₁ to C₂₅ alkyl group, or a cationic counterion;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group;
   or the pharmaceutically acceptable salt or ester thereof,
   wherein the stereochemistry at carbon a is either substantially R or substantially S.

In one aspect, when the compound has the formula VII, U comprises oxygen, Y¹ is hydrogen and Z is CHF, CF₂, or CH₂. In another embodiment, U comprises oxygen, Y¹ is hydrogen, Z is CHF, and W is oxygen. In a further embodiment, U comprises oxygen, Y¹ is hydrogen, Z is CHF, W is oxygen, V is not present, and R¹ comprises hydrogen or a branched or straight chain C₁ to C₂₅ alkyl group. In another embodiment, U comprises oxygen, Y¹ is hydrogen, Z is CHF, W is oxygen, V is not present, R¹ comprises hydrogen or a branched or straight chain C₁ to C₂₅ alkyl group, X¹ is hydrogen and X² is OH or OC(O)R³, wherein R³ comprises a branched or straight chain C₁ to C₂₅ alkyl group such as, for example, an oleate group or a palmitate group.

In one aspect, when the compound has the formula VII, U comprises oxygen, Z is CF₂ and W is oxygen. In another aspect, U comprises oxygen, Z is CF₂, W is oxygen, V is not present, and R¹ comprises hydrogen or a branched or straight chain C₁ to C₂₅ alkyl group. In a further aspect, U comprises oxygen, Z is CF₂, W is oxygen, V is not present, R¹ comprises hydrogen or a branched or straight chain C₁ to C₂₅ alkyl group, X¹ is hydrogen, and X² is OH or OC(O)R³, wherein R³ is a branched or straight chain C₁ to C₂₅ alkyl group such as, for example, an oleate group or a palmitate group.

In one aspect, when the compound has the formula VII, U comprises oxygen, Z is CHF or CF₂ and W is oxygen. In another aspect, U comprises oxygen, Z is CHF or CF₂, W is oxygen, V is oxygen, and R¹ comprises hydrogen or a branched or straight chain C₁ to C₂₅ alkyl group. In a further aspect, U comprises oxygen, Z is CHF or CF₂, W is oxygen, V is oxygen, R¹ comprises hydrogen or a branched or straight chain C₁ to C₂₅ alkyl group, X¹ is hydrogen, and X² is OH or OC(O)R³, wherein R³ is a branched or straight chain C₁ to C₂₅ alkyl group such as, for example, an oleate group or a palmitate group.

In one aspect, when the compound has the formula VII, U comprises oxygen, Z is CH₂ and W is oxygen. In another aspect, U comprises oxygen, Z is CH₂, W is oxygen, V is not present, and R¹ is hydrogen or a branched or straight chain C₁ to C₂₅ alkyl group. In a further aspect, U comprises oxygen, Z is CH₂, W is oxygen, V is not present, R¹ is hydrogen or a branched or straight chain C₁ to C₂₅ alkyl group, X¹ is hydrogen, and X² is OH or OC(O)R³, wherein R³ comprises a branched or straight chain C₁ to C₂₅ aikyl group such as, for example, an oleate group or a palmitate group.

In one aspect, when the compound has the formula VII, U comprises oxygen, Z is CH₂, W is oxygen, V is oxygen, and R¹ comprises hydrogen or a branched or straight chain C₁ to C₂₅ alkyl group. In another aspect, U comprises oxygen, X¹ is hydrogen and X² is a branched or straight chain C₁ to C₂₅ alkyl group such as, for example, an oleate group or a palmitate group.

In one aspect, when the compound has the formula VII, U comprises oxygen, Z is CH₂ and W is sulfur. In another aspect, U comprises oxygen, Z is CH₂, W is sulfur, V is not present, and R¹ is hydrogen or a branched or straight chain C₁ to C₂₅ alkyl group. In a further aspect, U comprises oxygen, Z is CH₂, W is sulfur, V is not present, and R¹ is hydrogen or a branched or straight chain C₁ to C₂₅ alkyl group, X¹ is hydrogen, and X² is OH or OC(O)R³, wherein R³ is a branched or straight chain C₁ to C₂₅ alkyl group such as, for example, an oleate group or a palmitate group.

In one aspect, when the compound has the formula VII, U comprises oxygen, Z is sulfur and W is oxygen. In another aspect, U comprises oxygen, Z is sulfur, W is oxygen, V is not present, and R¹ is hydrogen or a branched or straight chain C₁ to C₂₅ alkyl group. In a further aspect, U comprises oxygen, Z is sulfur, W is oxygen, V is not present, R¹ is hydrogen or a branched or straight chain C₁ to C₂₅ alkyl group, X¹ is hydrogen, and X² is OH or OC(O)R³, wherein R³ is a branched or straight chain C₁ to C₂₅ alkyl group such as, for example, an oleate group or a palmitate group.

In one aspect, when the compound has the formula VII, U comprises oxygen, Z is sulfur, W is oxygen, V is oxygen, and R¹ is hydrogen or a branched or straight chain C₁ to C₂₅ alkyl group. In a further aspect, Z is sulfur, U comprises oxygen, W is oxygen, V is oxygen, R¹ is hydrogen or a branched or straight chain C₁ to C₂₅ alkyl group, X¹ is hydrogen and X² is OH or OC(O)R³, wherein R³ is a branched or straight chain C₁ to C₂₅ alkyl group such as, for example, an oleate group or a palmitate group.

In another embodiment, the compounds having the formula VII are presented in Table 2 below. Where applicable, C₁₇ denotes C₁₇H₃₃.

TABLE 2

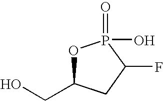

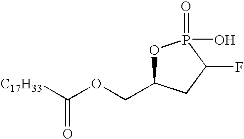

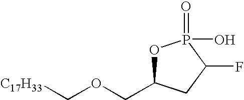

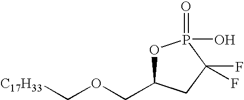

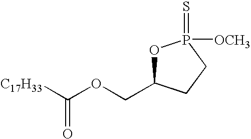

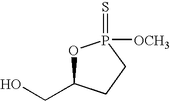

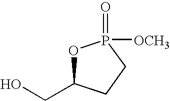

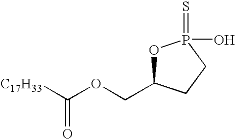

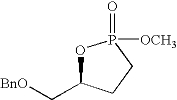

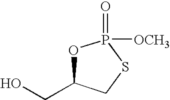

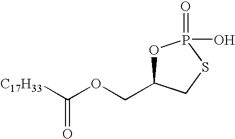

Any of the compounds described herein can be the pharmaceutically acceptable salt or ester thereof. Pharmaceutically acceptable salts are prepared by treating the free acid with an appropriate amount of a pharmaceutically acceptable base. Representative pharmaceutically acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. The molar ratio of compounds of structural formula I or VII to base used are chosen to provide the ratio desired for any particular salts. For preparing, for example, the ammonium salts of the free acid starting material, the starting material can be treated with approximately one equivalent of pharmaceutically acceptable base to yield a neutral salt.

Ester derivatives are typically prepared as precursors to the acid form of the compounds—as illustrated in the examples below—and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

II. Methods for Preparing LPA Analogs

In one aspect, described herein are methods for preparing compounds having the formula III 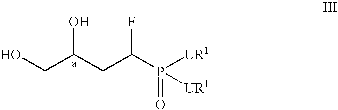
wherein each R¹ comprises, independently, hydrogen, a branched or straight chain C₁ to C₂₅ alkyl group, a cationic counterion, or both R¹ form a cyclic or heterocyclic group;
each U comprises, independently, oxygen, sulfur, or NR¹; and
the stereochemistry at carbon a is R or S, or the pharmaceutically acceptable salt or ester thereof. The method involves
(a) reacting a compound having the formula IV 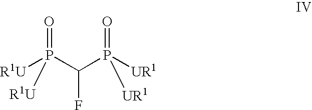
   with a compound having the formula V 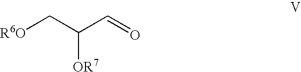
   wherein R⁶ and R⁷ are protecting groups,
   in the presence of a base;
(b) hydrogenating the compound produced in step (a); and
(c) deprotecting the compound produced in step (b) to produce a compound having the formula III.

The compound having the formula m can be prepared by treating (R₁O)₂(O)PCH₂P(O)(OR₁)₂ with a base followed by the addition of a fluorinating reagent. Any base that can deprotonate one of the hydrogen atoms present on the methylene group are suitable. Examples of bases include, but are not limited to hydrides such as sodium hydride. The fluorinating agent can be any compound that provides a source of electrophilic fluorine. Examples of fluorinating agents include, but are not limited to, Selectfluor (1-chloromethyl-4-fluoro-1,4-diazobicyclo[2.2.2]octane bis(tetrafluoroborate) (F-TEDA-BF₄)) and N-fluorodibenzenesulfonimide.

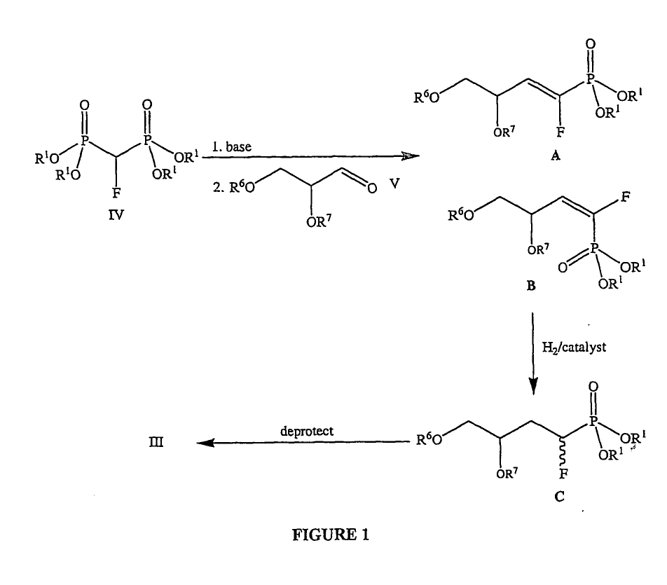

In step: (a), compounds IV and V react with one another in the presence of a base. The order at which compound IV, V, and the base are added to one another can vary. In one aspect, the compound having the formula IV is reacted with a base to produce a carbanion species. Any base that can deprotonate the CHF proton in formula IV is suitable. Examples of bases include organolithium compounds such as, for example, n-butyllithium. In this aspect, after the carbanion species is produced, aldehyde compound V is added and condenses with the carbonion species. The condensation product is shown in FIG. 1, where two isomers (A and B) are shown. The two isomers can be separated using techniques known in the art such as, for example, by column chromatography. The protecting groups R⁶ and R⁷ can be any of those disclosed in Protective Groups in Organic Synthesis by T. W. Green, John Wiley and Sons, 1981, which is incorporated by reference in its entirety. R⁶ and R⁷ they can be the same or different. In one aspect, R⁶ and R⁷ together form a ring. For example, the compound having the formula VI can be used.

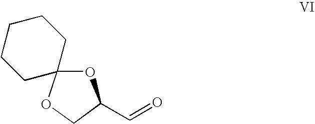

By controlling the stereochemistry of the aldehyde compound V, it is possible to control the stereochemistry at carbon a in formula III. For example, if the aldehyde compound VI is used in step (a), the stereochemistry at carbon a of formula III will be S.

Step (b) involves hydrogenating the alkene group of the condensation product produced after step (a). The reaction generally involves exposing the condensation product to hydrogen in the presence of a catalyst. Numerous hydrogenation catalysts are known in the art. In one aspect, the catalyst is Pd-C. The hydrogenation product is depicted as compound C in FIG. 1. In another aspect, asymmetric hydrogenation catalysts can be used in step (b). In this aspect, the resultant hydrogenation product can be substantially one enantiomer or diastereorner. The use of asymmetric hydrogenation catalysts are know in the in the art. Examples of asymmetric hydrogenation catalysts useful in the methods described herein include, but are not limited to, the catalysts shown below.

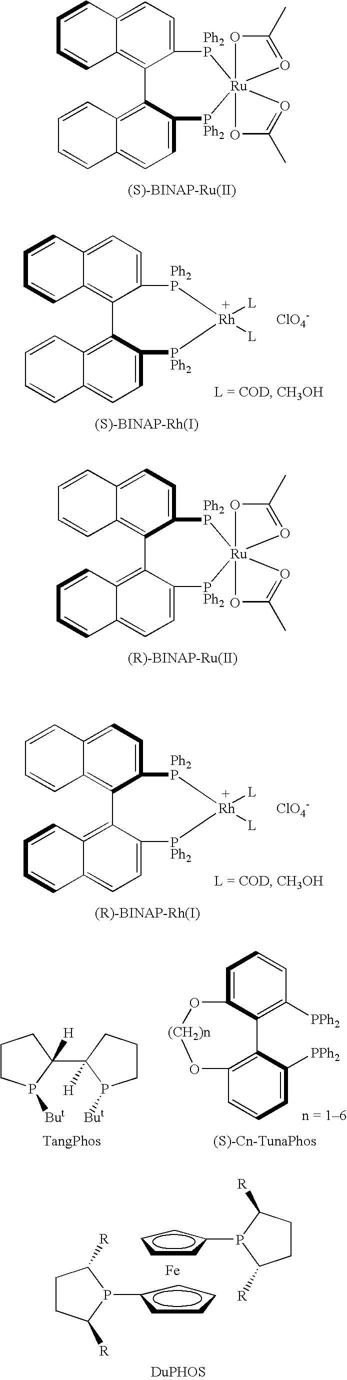

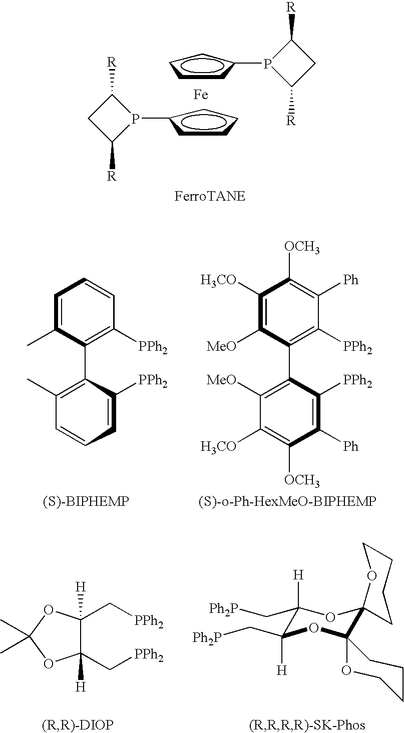

After the hydrogenation step (b), the protecting groups R⁶ and R⁷ are removed. The deprotection can be performed using techniques known in the art. For example, the techniques disclosed in Protective Groups in Organic Synthesis by T. W. Green, John Wiley and Sons, 1981, which is incorporated by reference in its entirety, are useful. In one aspect, a catalytic amount of an acid such as, for example, p-touenesulfonic acid, can be used. Depending upon the identity of R⁶ and R⁷, one or both of R⁶ and R⁷ can be removed (i.e., deprotected). Removal of R⁶ and R⁷ produces the diol compound III (FIG. 1).

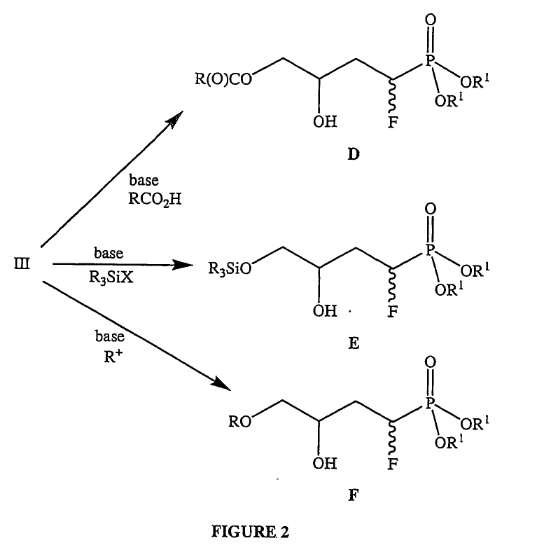

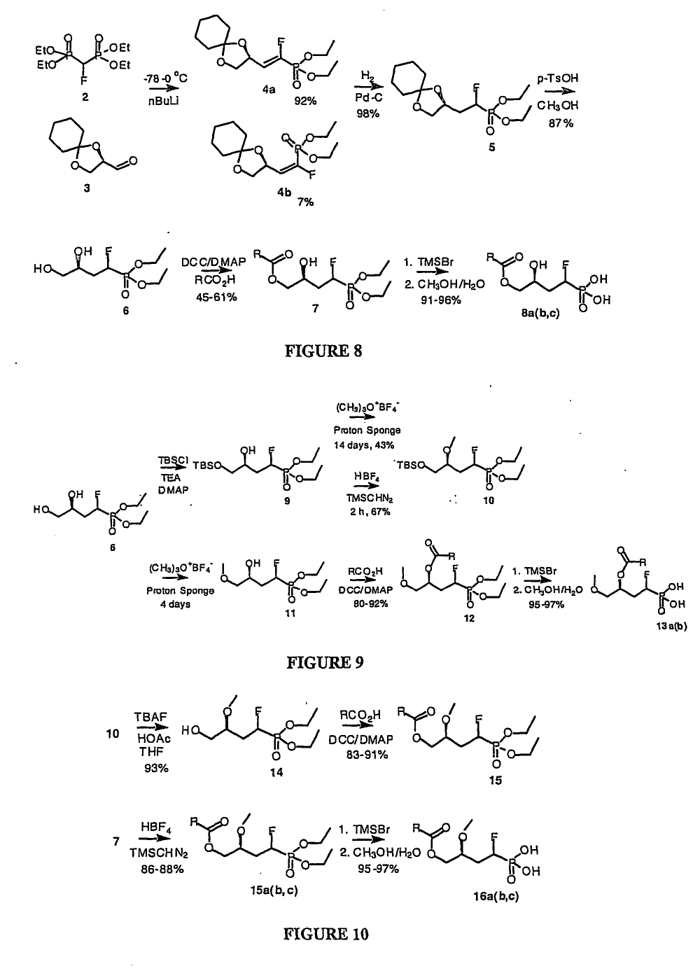

The diol compound III can be converted to numerous other compounds using techniques known in the art. In one aspect, reacting the diol compound III with a base followed by a carboxylic acid can convert the primary hydroxyl group to the corresponding ester D (FIG. 2). In another aspect, the diol compound III can be treated with a base followed by the addition of an organosilane or alkylating agent to convert the primary hydroxyl group to the corresponding silyl or alkoxy compounds E and F, respectively. Once the primary hydroxyl group is protected, the secondary hydroxyl group can be converted to another functional group such an alkoxy or ester group. Depicted in FIGS. 8-10 are various, specific reaction sequences for protecting and deprotecting the hydroxyl groups of compound III. Specific procedures are shown below.

In another aspect, compounds having the formula VII can be prepared by reacting a compound having the formula VIII 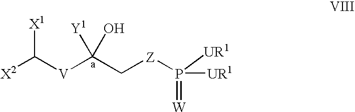
wherein
X¹, X², and Y¹ comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;

R² comprises hydrogen, a branched or straight chain C₁ to C₂₅ alkyl group, a cycloalkyl group, a heterocycloalkyl group, an aryl group, a heteroaryl group or a protecting group;

R³ comprises a branched or straight chain C₁ to C₂₅ alkyl group, a cycloalkyl group, a heterocycloalkyl group, an aryl group, a heteroaryl group;

or the pharmaceutically acceptable salt or ester thereof,

wherein the stereochemistry at carbon a is either substantially R or substantially S, with a dehydrating agent

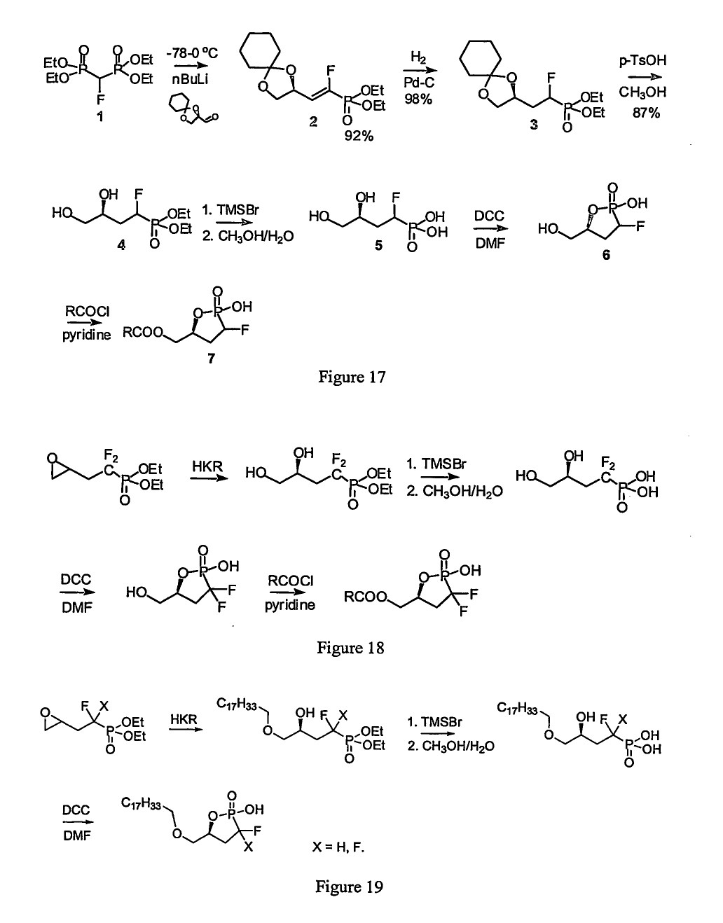

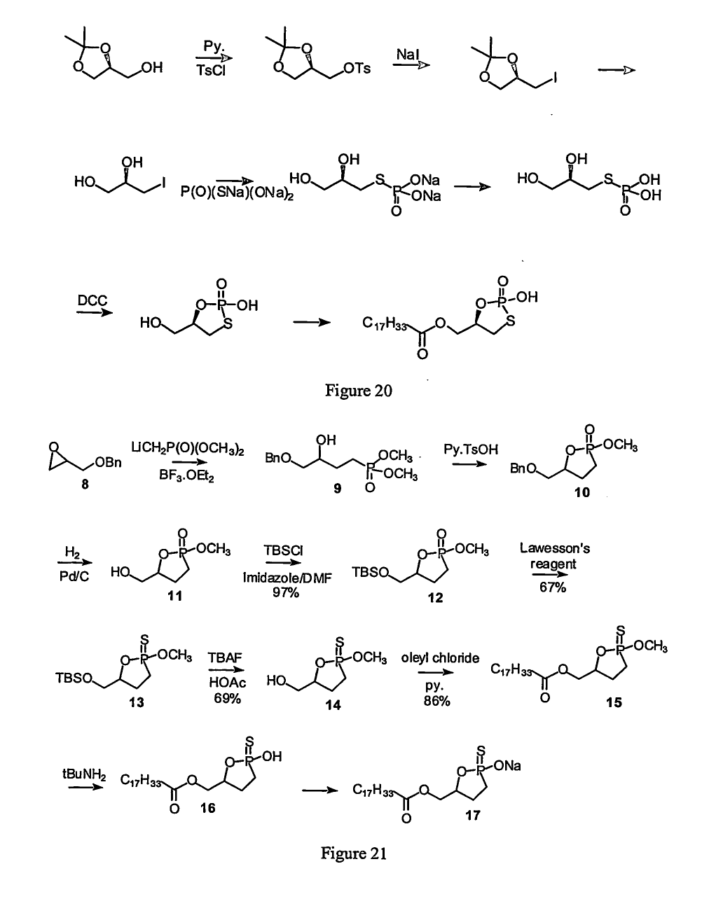

The dehydrating agent facilitates the cyclization of the compound having the formula VIII to produce cyclic compound VII, which produces water or an alcohol depending upon the identity of R¹. Using techniques known in the art and described herein, it is possible to control the stereochemistry of the hydroxyl group at carbon a. Examples of dehydrating agents include, but are not limited to, acids such as, for example, Lewis acids (organic acids) or Bronsted acids. In another aspect, the dehydrating agent includes dicyclohexylcarbodiimide (DCC) or p-toluenesulfonic acid. Once the cyclic compound VII is produced, the compound can undergo further chemical manipulations known in the art. For example, when W is oxygen in formula VII, the P═O group can be converted to a P═S group by reacting the cyclic compound possessing the P═O group with a compound such as, for example, Lawesson's agent. In one aspect, the reaction schemes depicted in FIGS. 17-22 can be used to synthesize and derivatize the cyclic compounds described herein.

III. Pharmaceutical Compositions

In one aspect, any of the compounds having the formula I can be combined with at least one pharmaceutically-acceptable carrier to produce a pharmaceutical composition. The pharmaceutical compositions can be prepared using techniques known in the art In one aspect, the composition is prepared by admixing the compound having the formula I with a pharmaceutically-acceptable carrier. The term “admixing” is defined as mixing the two components together so that there is no chemical reaction or physical interaction. The term “admixing” also includes the chemical reaction or physical interaction between the compound having the formula I and the pharmaceutically-acceptable carrier.

Pharmaceutically-acceptable carriers are known to those skilled in the art. These most typically would be standard carriers for administration to humans, including solutions such as sterile water, saline, and buffered solutions at physiological pH.

Molecules intended for pharmaceutical delivery may be formulated in a pharmaceutical composition. Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally).

Preparations for administration include sterile aqueous or non-aqueous solutions, suspensions, and emulsions. Examples of non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

It will be appreciated that the actual preferred amounts of active compound in a specified case will vary according to the specific compound being utilized, the particular compositions formulated, the mode of application, and the particular situs and mammal being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (1999).

IV. Methods of Use

LPA is an important lysophospholipid mediator produced by activated platelets. LPA elicits a variety of biological effects, which includes platelet aggregation, smooth muscle contraction, changes in cell morphology, and stimulation of cell growth and proliferation. Moreover, the observation that LPA is the key cell proliferation factor overproduced in ascites of human ovarian cancer patients has led to the validation of the G-protein-coupled seven-transmembrane domain LPA receptors as targets for cancer therapy. In addition, phosphatidic acid (PA), the product of the action of phospholipase D on phosphatidylcholine and other phospholipids, is well-established as an important intermediate in the biosynthesis of phosphoglycerides as a regulator of phosphoinositide metabolism, in physiological processes from cell growth to protein trafficking.

The compounds described herein possess improved properties over LPA. For example, the compounds described herein have prolonged biological activity by altering pharmocokinetics, metabolism, and ligand binding.

In one aspect, the compounds described herein can be used as long-lasting agonists, antagonists, or enzyme inhibitors.

In one aspect, the compounds described herein are a PPARγ agonist For example, the compounds described herein can stimulate PPAR-responsive element reporter expression, the endogenous PPARγ-controlled gene CD36, and induce monocyte lipid accumulation from oxidized LDL via the CD36 scavenger receptor. The techniques disclosed in McIntyre et al. Proc. Nat. Acad. Sci. 100, pp 131-136, January 2003, which is incorporated by reference in its entirety, can be used to determine if the compounds described herein can be used as PPARγ agonists.

In another aspect, the compounds described herein can inhibit lipid phosphatase activity, lipid kinase, or phopholipase in order to treat or prevent a disease in a subject.

In one aspect, described herein are methods for improving wound healing in a subject in need of such improvement by contacting the wound of a mammal with any of the compounds described herein. The compounds or pharmaceutical compositions described herein can be delivered onto cells, tissues, and/or organs, for example, by injection, spraying, squirting, brushing, painting, coating, and the like. Delivery can also be via a cannula, catheter, syringe with or without a needle, pressure applicator, pump, and the like. In one aspect, any of the compounds described herein can be incorporated into a sponge, dressing, bandage, hydrogel, or cream in order to enhance wound healing.

In another aspect, described herein are methods for treating or preventing in a subject a disease by administering to the subject any of the compounds described herein. Examples of diseases treated by the compounds described herein include, but are not limited to, cancer and diabetes. In one aspect, the compounds described herein can be used to treat ovarian cancer.

In a further aspect, described herein are methods for reducing inflammation or an allergic response in a subject by administering to the subject the compound any of the compounds described herein. In another aspect, described herein are methods for increasing or altering cardiovascular function in a subject by administering to the subject any of the compounds described herein. For example, the compounds can vasodilate or vasoconstrict blood vessels depending upon the selection of the compound.

In another aspect, described herein are methods for eliciting or inhibiting platelet aggregation in a subject by administering to the subject any of the compounds described herein.

In an additional aspect, described herein are methods for maintaining or terminating embryonic development in a subject by administering to the subject any of the compounds described herein.

Described herein are methods for determining the activity of lysophosphatidic acid or phosphatidic acid. The method involves (a) measuring the activity of any of the compounds described herein; and (b) measuring the same activity of lysophosphatidic acid or phosphatidic acid.

In one aspect, when a compound having the formula I has an acyl group, a reporter group is present on the acyl group. In one aspect, the reporter group is attached to the acyl group via a tether. Examples of reporter groups include, but are not limited to, a fluorescent tag, a radiolabel, a targeting moiety, a lipid, a peptide, a radionuclide chelator with a radionuclide, a spin-label, a glass surface, a plastic surface, or a combination thereof. Examples of fluorescent groups include, but are not limited to, BODIPY, fluorescein, or NBD-hexanoyl. Examples of radiolabels include, but are not limited to, 125I-tyrosine, 3H-acetyl, or ¹⁴C-acetyl. Examples of targeting moieties include, but are not limited to, 6-aminohexanoyl (Z) derivatives of integrin targeting peptide, such as ZYRGDS, Z-tat decapeptide for cell penetration, Z-GFLG for lysosome targeting, or HA oligosaccharide for CD-44 cancer targeting. Examples of spin labels include, but are not limited to, proxyl or doxyl groups. Examples of glass surfaces include, but are not limited to, glass silanized with an epoxy, activated ester, or thiol-reactive electrophilic functional groups, beads, or coverslips. Examples of plastics include, but are not limited to, plasma-etched polypropylene, chemically-modified polystyrene, or any other plastic material. In this aspect, the LPA analog having a reporter group can be used to target discovery of diseases, which can ultimately lead to drug discovery.

In another aspect, the compounds described herein can be used to maintain, increase, or inhibit cell growth or proliferation in cultures. In this aspect, the compounds can be used in tissue engineering.

In another aspect, the compounds described herein can be used to identify edg and non-edg receptor cites.

The following is a partial list of the many activities that can be determined in the present screening method:
1. Receptor agonist/antagonist activity:

A compendia of examples of specific screens for measuring these activities can be found in: “The RBI Handbook of Receptor Classification and Signal Transduction” K. J. Watling, J. W. Kebebian, J. L. Neumeyer, eds. Research Biochemicals International, Natick, Mass., 1995, and references therein. Methods of analysis can be found in: T. Kenakin “Pharmacologic Analysis of Drug-Receptor Interactions” 2nd Ed. Raven Press, New York, 1993, and references therein. In one aspect, agonists or antagonists of lysophosphatidic acid binding to or activating lysophosphatidic acid receptors of the edg class in a cell.
2. Enzyme inhibition:

A compendia of examples of specific screens for measuring these activities can be found in: H. Zollner “Handbook of Enzyme Inhibitors”, 2nd Ed. VCH Weinheim, FRG, 1989, and references therein.
3. Central nervous system, autonomic nervous system (cardiovascular and gastrointestinal tract), antihistaminic, anti-inflammatory, anaesthetic, cytotoxic, and antifertility activities:

A compendia of examples of specific screens for measuring these activities can be found in: E. B. Thompson, “Drug Bioscreening: Drug Evaluation Techniques in Pharmacology”, VCH Publishers, New York, 1990, and references therein.
4. Anticancer activities:

A compendia of examples of specific screens for measuring these activities can be found in: I. J. Fidler and R. J. White “Design of Models for Testing Cancer Therapeutic Agents”, Van Nostrand Reinhold Company, New York, 1982, and references therein.
5. Antibiotic and antiviral (especially anti-HIV) activities:

A compendia of examples of specific screens for measuring these activities can be found in: “Antibiotics in Laboratory Medicine”, 3rd Ed., V. Lorian, ed. Williams and Wilkens, Baltimore, 1991, and references therein. A compendia of anti-HIV screens for measuring these activities can be found in: “HIV Volume 2: Biochemistry, Molecular Biology and Drug Discovery”, J. Karn, ed., IRL Press, Oxford, 1995, and references therein.
6. Immunomodulatory activity:

A compendia of examples of specific screens for measuring these activities can be found in: V. St. Georgiev (1990) “Immunomodulatory Activity of Small Peptides” Trends Pharm. Sci. 11, 373-378.
7. Pharmacokinetic properties:

The pharmacological activities assayed in the screening method include half-life, solubility, or stability, among others. For example, methods of analysis and measurement of pharmacokinetic properties can be found in: J.-P. Labaune “Handbook of Pharmacokinetics: Toxicity Assessment of Chemicals”, Ellis Horwood Ltd., Chichester, 1989, and references therein.

The compounds described herein are stable when compared to LPA. For example, acyl migration occurs in LPA, which complicates studies of positional specificity. By testing any of the compounds described herein, it is possible to identify potential activities of LPA. Once the potential activity has been identified, it is possible to test the activity with LPA. Thus, the compounds described herein are useful tools in determining other potential activities of LPA, which will ultimately lead to the treatment or prevention of additional diseases.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

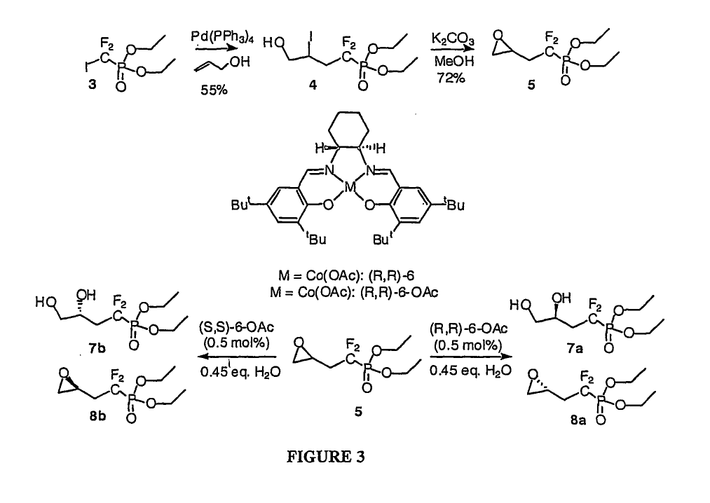

I. Synthesis of α-Difluoro-Analogs of LPA One approach to the synthesis of difluoromethylene anlogs of LPA is depicted in FIG. 3. The addition reaction of diethyl iododifluoromethylenephosphonate 3 to allyl alcohol catalyzed by tetrakis(triphenylphosphine)-palladium in hexane gave the corresponding iodohydrin 4 in 79% yield. However, treatment of the iodohydrin 4 with diluted K₂CO₃/MeOH solution for 5 min at room temperature provided the desired epoxide 5 in good yield (72%). Next, terminal epoxide 5 was employed for the HKR reaction, constituting the first application of HKR in a substrate containing both fluorine and phosphonate functionalities. Few examples of HKR with fluorine-containing epoxides were found, and no HKR substrates have been reported for phosphonate or phosphate-containing epoxides. The reaction of racemic epoxide with 0.45 equiv of H₂O in a minimum volume of THF in the presence of 1.0 mol % of (R,R)-6-OAc gave diol 7a in 99% ee and 69% isolated yield. Similarly, catalyst (S,S)-6-OAc provided the opposite configuration of diol 7b in 99% ee and 70% yield. The epoxide and diol were readily separated by flash chromatography, providing an excellent example of the scope and utility of the HKR process.

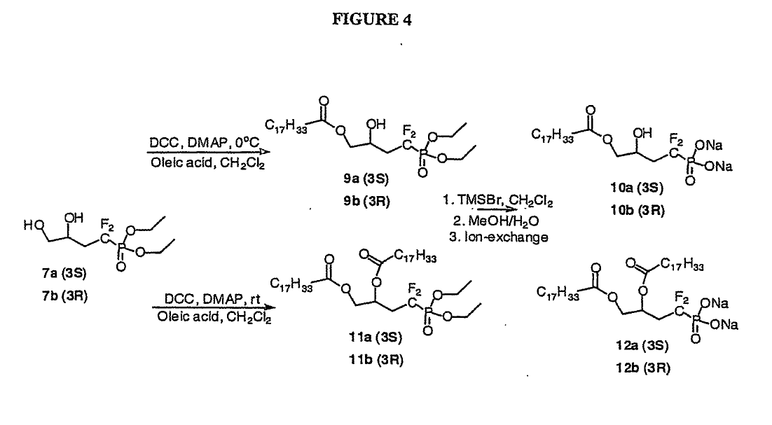

Regioselective acylation at the primary hydroxyl of the 1,2-diol was readily accomplished. Thus, treatment of 7a with 0.95 equiv of oleic acid and 1.2 equiv DCC and DMAP in CH₂Cl₂ at 0° C. gave 9a in 42% yield after chromatography, accompanied by a small amount of diester (FIG. 4). When the reaction was performed at rt, the ratio of primary ester to diester decreased. Diesters bearing identical acyl chains, e.g., 11a and 11b, could be obtained in 73% yield, with 2.4 equiv of oleic acid in the presence of excess DCC and DMAP in CH₂Cl₂. Dealkylation of phosphonic acid diethyl esters was achieved by treatment with excess bromotrimethylsilane (10.0 equiv) for 8 hr at rt; interestingly, use of only 3.0 equiv of TMSBr did not result in complete dealklylation. After hydrolysis by aqueous methanol (95%) followed by ion exchange chromatography, the sodium salts of LPA analogues 10 and PA analogues 12 were obtained in essentially quantitative yield.

The enantiomeric purity of diols 7a and 7b was determined by Mosher's ester method, and optical purities were measured by integration of the ¹H NMR. The double doublet at δ 4.35 ppm in 12a was shifted to δ 4.44 ppm in 12b. There was no detectable signal at δ 4.44 ppm in 12a, nor at δ 4.35 ppm in 12b, indicating that each diol had been obtained in >99% ee.

General Procedure. Chemicals were obtained from Aldrich and Acros and were used without prior purification. Solvents used were of reagent grade and were distilled before use: THF was distillated from sodium wire, and methylene chloride was distillated from CaH₂. Reactions were performed under an inert atmosphere (N₂ or Ar) unless otherwise indicated. ¹H and ¹³C spectra were recorded at 25° C. at 400 MHz (¹H), 101 MHz (¹³C), 162 MHz (³¹P) and 376 MHz (¹⁹F). Chemical shifts are given in ppm with TMS as internal standard (δ=0.00); ³¹P, 85% H₃PO₄ (δ=0.00); ¹⁹F, CFCl₃ (δ=0.00). Optical Rotations were measured on Perlin Elmer 343 Polarimeter.

Diethyl [1,1-difluoro-3-iodo-4-hydroxy-butyl]phosphonate 4. To a stirred solution of Pd(PPh₃)₄ (0.718 g, 0.621 mmol, 0.026 eq.) and allyl alcohol (2.774 g, 47.76 mmol) in hexane (20 mL) at rt was added diethyl iododifluoromethylphosphonate (7.499 g, 23.88 mmol), and the resultant mixture was stirred for 10 min. The reaction mixture was dissolved in 100 mL of hexane/ethyl acetate (1:1). The resulting solid was removed by filtrate and the solid was washed with hexane/ethyl acetate solvent. The combined solution were then concentrated to give a residue which was purified by flash chromatograph on silica gel (HE:AE=1:1, R_{f}=0.26) gave a colorless liquid (7.010 g, 18.844 mmol, 79%). ¹H NMR (CDCl₃): 4.48 (m, 1H), 4.27 (m, 4H), 3.75 (d, J=5.2 Hz, 2H), 2.98 (m, 1H), 2.71 (m, 1H), 2.01 (br, 1H), 1.36 (m, 6H). ¹³C NMR (CDCl₃): 119.79 (td, J=262.36, 215.50 Hz), 67.94 (s), 64.86 (dd, J=9.96, 3.12 Hz), 40.36 (td, J=19.91, 16.09 Hz), 23.54 (s), 16.27 (d, J=5.33 Hz). ¹⁹F NMR (CDCl₁₃): −110.77 (1F, dddd, J=297.29, 105.37, 39.51, 13.17 Hz), −112.03 (1F, dddd, J=297.29, 105.37, 39.51, 13.17 Hz). ³¹P NMR (CDCl₃): 6.94 (t, J=105.41 Hz).

Diethyl [1,1-difluoro-3,4epoxy-butyl]phosphonate 5. K₂CO₃ (0.245 g, 1.774 mmol) was added to a solution of compound 4 (0.110 g, 0.296 mmol) in MeOH (15 mL). The reaction mixture was stirred for 10 min at rt and then diluted with water (15 mL) and extracted with CH₂Cl₂ (20 mL×3). The organic phase was dried (Na₂SO₄), filtrated, and concentrated in vacuo. The residue was purified by flash column chromatograph to give epoxide as a colorless oil (52 mg, 0.213 mmol, 72%, HB:AE=1:1, R_{f}=0.27). ¹H NMR (CDCl₃): 4.25 (m, 4H), 3.20 (m, 1H), 2.80 (t, J=4.5 Hz, 1H), 2.53 (dd, J=2.4, 7.6 Hz, 1H), 2.37 (m, 1H), 2.17 (m, 1H), 1.35 (t, J=7.2 Hz, 6H). ¹³C NMR (CDCl₃): 119.79 (td, J=262.36,215.50 Hz), 64.62 (d, J=6.84 Hz), 46.24 (s), 45.54 (dd, J=13.88, 6.94 Hz), 37.92 (m), 16.32 (d, J=5.03 Hz). ¹⁹F NMR (CDCl₃): −110.40 (1F, dddd, J=302.56, 105.37, 21.07, 17.31 Hz), −111.48 (1F, dddd, J=302.56, 105.37, 21.07, 17.31 Hz). ³¹P NMR (CDCl₃): 7.24 (t, J=105.41 Hz). MS (CI) m/z 245.0 (M⁺+1, 100.00). HRMS, M⁺+1, Found: 245.0751. Calcd for C₈H₁₆F₂O₅P, 245.0754.

Hydrolytic Kinetic Resolution of Epoxide 5 with (R,R) catalyst. A 10 mL flask equipped with a stir bar was charged with (R,R)-1 (20.2 mg, 33 μmol, 0.01 equiv). The catalyst was dissolved in 0.4 mL of PhMe and treated with AcOH (8 μL, 0.132 mmol). The solution was allowed to stir at room temperature open to air for 30 min over which time the color changed from orange-red to a dark brown. The solution was concentrated in vacuo to leave a crude brown solid. The resulting catalyst residue was dissolved in epoxide (0.816 g, 3.344 mmol) and THF (120 μL) at room temperature, the reaction flask was cooled to 0° C., and H₂O (27.1 μL, 1.505 mmol, 0.45 equiv) was added dropwise over 5 min. The reaction was allowed to warm to room temperature and stir 14 h. Chromatograph on silicon gel get (R)-epoxide (0.400 g, 1.637 mmol, 98%, R_{f}=0.27, HE:AE=1:1) and (S)-diol (0.302 g, 1.154 mmol, 69%, R_{f}=0.27, AE). The ee value of the diol was determined to be >99.0% by Mosher ester.

Diethyl [1,1-Difluoro-3 (S), 4-dihydroxybutyl]phosphonate 7a. Colorless liquid. ¹H NMR (CDCl₃): 4.24 (m, 4H), 4.10 (m, 1H), 3.62 (dd, J=10.8, 3.6 Hz, 1H), 3.49 (dd, J=10.8, 6.0 Hz, 1H), 2.21 (m, 2H), 1.35 (m, 6H). ¹³C NMR (CDCl₃): 120.17 (td, J=260.45, 215.20 Hz), 66.26 (s), 65.97 (m), 65.04 (dd, J=24.54, 6.94 Hz), 39.10 (m), 16.29 (d, J=5.43 Hz). ¹⁹F NMR (CDCl₃): −106.69 (1F, ddt, J=302.56, 103.86, 16.93 Hz), −111.10 (1F, ddt, J=302.56, 103.86, 16.93 Hz). ³¹P NMR (CDCl₃): 8.39 (t, J=107.51 Hz). MS (CI) m/z 263.1 (M⁺+1,100.00), 217.0 (M+−C₃H₈, 3.59). HRMS, M⁺+1, Found: 263.0876. Calcd for C₈H₁₈F₂O₅P, 263.0860. [α]²⁰_{D}=−10.39 (c=0.38, MeOH).

Diethyl [1,1-difluoro-3(R)-3,4-epoxy-butyl]phosphonate 8a. Colorless liquid. ¹H NMR (CDCl₃): 4.22 (m, 4H), 3.15 (m, 1H), 2.77 (dd, J=4.8, 4.0 Hz, 1H), 2.49 (dd, J=4.4, 2.0 Hz, 1H), 2.33 (m, 1H), 2.14 (m, 1H), 1.32 (m, 6H). ¹³C NMR (CDCl₃): 119.52 (td, J=260.75, 216.20 Hz), 64.56 (d, J=6.84 Hz), 46.15 (s), 45.45 (m), 37.86 (m), 16.24 (d, J=6.13 Hz). ¹⁹F NMR (CDCl₃): −110.48 (1F, dddd, J=302.56, 105.37, 21.07, 15.81 Hz), −111.41 (1F, dddd, J=302.56, 105.37, 21.07, 15.81 Hz). ³¹P NMR (CDCl₃): 7.21 (t, J=105.41 Hz). [α]²⁰_{D}+6.53 (c=1.50, MeOH).

Hydrolytic Kinetic Resolution of Epoxide 5 with (S,S) catalyst. A 10 mL flask equipped with a stir bar was charged with (S,S)-1 (27.7 mg, 46 μmol, 0.01 equiv). The catalyst was dissolved in 0.5 mL of PhMe and treated with AcOH (10 μL, 0.183 mmol). The solution was allowed to stir at room temperature open to air for 30 min over which time the color changed from orange-red to a dark brown. The solution was concentrated in vacuo to leave a crude brown solid. The resulting catalyst residue was dissolved in epoxide (1.119 g, 4.586 mmol) and THF (150 μL) at room temperature, the reaction flask was cooled to 0° C., and H₂O (37.2 μL, 2.064 mmol, 0.45 equiv) was added dropwise over 5 min. The reaction was allowed to warm to room temperature and stir 14 h. Chromatograph on silicon gel get (S)-epoxide (0.549 g, 2.250 mmol, 98%) and (S)-diol (0.422 g, 1.611 mmol, 70%). The ee of the diol was determined to be >99.0% by Mosher ester.

Diethyl [1,1-Difluoro-3 (R), 4dihydroxybutyl]phosphonate 7b. Colorless liquid. ¹H NMR (CDCl₃): 4.29-4.22 (m, 4H), 4.08 (m, 1H), 3.77 (br, 2H), 3.60 (dd, J=11.2, 3.6 Hz, 1H), 3.47 (dd, J=11.2, 6.4 Hz, 1H), 2.29-2.12 (m, 2H), 1.33 (m, 6H). ¹³C NMR (CDCl₃): 120.14 (td, J=260.05, 214.80 Hz), 66.22 (s), 65.97 (m), 65.00 (dd, J=22.22, 6.94 Hz), 38.89 (td, J=19.91, 15.29 Hz), 16.25 (d, J=5.33 Hz). ¹⁹F NMR (CDCl₃): −107.01 (1F, ddt, J=302.56, 105.37, 17.31 Hz), −111.09 (1F, ddt, J=302.56, 105.37, 17.31 Hz). ³¹P NMR (CDCl₃): 8.29 (dd, J=110.75, 105.41 Hz). [α]²⁰_{D}=+9.98 (c=0.48, MeOH).

Diethyl [1,1-difluoro-3(S)-3,4-epoxy-butyl]phosphonate 8b. Colorless liquid. ¹H NMR (CDCl₃): 4.22 (m, 4H), 3.15 (m, 1H), 2.77 (dd, J=4.8, 4.0 Hz, 1H), 2.49 (dd, J=4.4, 2.0 Hz, 1H), 2.33 (m, 1H), 2.14 (m, 1H), 1.32 (m, 6H). ¹³C NMR (CDCl₃): 119.52 (td, J=260.75, 216.20 Hz), 64.56 (d, J=6.84 Hz), 46.15 (s), 45.45 (m), 37.86 (m), 16.24 (d, J=6.13 Hz). ¹⁹F NMR (CDCl₃): −110.48 (1F, dddd, J=302.56, 105.37, 21.07, 15.81 Hz), −111.41 (1F, dddd, J=302.56, 105.37, 21.07, 15.81 Hz). ³¹P NMR (CDCl₃): 7.21 (t, J=105.41 Hz). [α]²⁰_{D}=−6.11 (c=0.72, MeOH).

Diethyl [1,1-Difluoro-3(S)-hydroxyl4-(oleoyl)butyl]phosphonate 9a. To a solution of diol (67 mg, 0.256 mmol) and oleic acid (68 mg, 0.243 mmol) in dry CH₂Cl₂ (1 mL) was added a solution of DCC (63 mg, 0.307 mmol) and DMAP (9 mg, 0.154 mmol) in dry CH₂Cl₂ (1 mL) at 0° C. The solution was stirred for 16 h at 0° C., filtered, concentrated in vacuo, and the residue was purified on silica gel (n-hexane/ethyl acetate, HE:AE=2:1, R_{f}=0.17 ) to afford ester (56 mg, 0.108 mmol, 42%) as a waxy solid. ¹H NMR (CDCl₃): 5.32 (m, 2H), 4.32-4.24 (m, 5H), 4.09 (d, J=5.2 Hz, 2H), 3.82 (br, 1H), 2.32 (m, 2H), 2.22 (m, 2H), 1.97 (m, 4H), 1.58 (t, J=7.2 Hz, 2H), 1.38 (m, 6H), 1.27 (m, 20H), 0.85 (t, J=7.2 Hz, 3H). ¹³C NMR (CDCl₃): 173.66 (s), 129.98 (s), 129.72 (s), 67.23 (s), 65.08 (dd, J=33.79, 6.94 Hz), 63.98 (m), 39.76 (td, J=19.21, 16.09 Hz), 34.07 (s), 31.88 (s), 29.74 (s), 29.67 (s), 29.50 (s), 29.20 (s), 29.14 (s), 29.08 (s), 27.19 (s), 27.14 (s), 24.87 (s), 22.66 (s), 16.35 (d, J=5.43 Hz), 14.08 (s). ¹⁹F NMR (CDCl₃): −106.19 (1F, ddt, J=304.07, 102.74, 15.81 Hz), −111.43 (1F, ddt, J=304.07, 102.74, 15.81 Hz). ³¹P NMR (CDCl₃): 8.42 (dd, J=109.78, 101.04 Hz). [α]²⁰_{D}=−1.67 (c=0.12, MeOH).

Diethyl [1,1-Difluoro-3 (R)-hydroxyl-4-(oleoyl)butyl]phosphonate 9b. Colorless liquid. ¹H NMR (CDCl₃): 5.32 (m, 2H), 4.32-4.23 (m, 5H), 4.08 (d, J=4.8 Hz, 2H), 3.83 (br, 1H), 2.32 (m, 2H), 2.23 (m, 2H), 1.97 (m, 4H), 1.60 (t, J=7.2 Hz, 2H), 1.37 (t, J=7.2 Hz, 6H), 1.25 (m, 20H), 0.85 (t, J=7.2 Hz, 3H). ¹³C NMR (CDCl₃): 173.65 (s), 129.96 (s), 129.70 (s), 120.17 (td, J=260.45, 215.20 Hz), 67.22 (s), 65.06 (dd, J=32.98, 7.64 Hz), 63.96 (m), 39.71 (td, J=19.91, 16.09 Hz), 34.07 (s), 31.86 (s), 29.73 (s), 29.66 (s), 29.48 (s), 29.28 (s), 29.13 (s), 29.06 (s), 27.18 (s), 27.13 (s), 24.85 (s), 22.64 (s), 16.33 (d, J=5.43 Hz), 14.06 (s). ¹⁹F NMR (CDCl₃): −106.28 (1F, ddt, J=302.94, 101.98, 16.18 Hz), −111.43 (1F, ddt, J=302.94, 101.98, 16.18 Hz). ³¹P NMR (CDCl₃): 8.40 (dd, J=109.78, 102.17 Hz). MS (CI) m/z 527.1 (M⁺+1, 12.66), 481.1 (M⁺—OC₂H₅, 100.00). HRMS, M⁺+1, Found: 527.3319. Calcd for C₂₆H₅₀F₂O₆P, 527.3316. [α]²⁰_{D}=+1.36 (c=0.22, MeOH).

Sodium [1,1-Difluoro-3 (S)-hydroxyl-4-(oleoyl)butyl]phosphonate 10a.

Thoroughly dried diethyl precursor 9a (30 mg, 0.057 mmol, 5 h under high vacuum) was dissolved in anhydrous methylene chloride (0.2 mL) at room temperature. Bromotrimethylsilane (38 μL, 0.290 mmol) was added with a dry syringe and stirred 4 h. TLC indicated that all of the reactant had disappeared, then the solvent removed under reduced pressure and dried under vacuum. The residue was dissolved in 95% methanol (1 mL) for 1 h and concentrated in vacuuo got colorless oil, which made a cloudy solution when dissolved in water. The water turned to clear after added 1-2 drops triethylamine (PH=7-8). This solution was absorbed to a sodium ion-exchange column (DOWEX 50WX8-200 resin, neutral Na⁺ form), and eluted with water. The fraction was lyophilized to give a colorless liquid (28 mg, 0.055 mmol, 96%). ¹H NMR (CD₃OD): 5.28 (m, 1H), 5.16 (m, 2H), 3.49 (dd, J=11.2, 4.8 Hz, 1H), 3.40 (dd, J=11.2, 5.2 Hz, 1H), 2.33 (m, 2H), 2.16 (td, J=7.2, 1.6 Hz, 2H), 1.84 (m, 4H), 1.44 (m, 2H), 1.15-1.11 (m, 20H), 0.72 (t, J=6.6 Hz, 3H). ¹³C NMR (CD₃OD): 174.04 (s), 130.88 (s), 130.79 (s), 67.71 (s), 39.72 (td, J=19.91, 16.09 Hz), 35.22 (s), 35.08 (s), 33.06 (s), 30.84 (s), 30.78 (s), 30.61 (s), 30.45 (s), 30.35 (s), 30.26 (s), 30.16 (s), 30.12 (s), 28.13 (s), 25.90 (s), 23.74 (s), 14.47 (s). ¹⁹F NMR (CD₃OD): −113.96 (m). ³¹P NMR (CDCl₃): 5.74 (dd, J=102.01 Hz). [α]²⁰_{D}=+4.83 (c=0.60, MeOH).

Sodium [1,1-Difluoro-3 (R)-hydroxyl-4-(oleoyl)butyl]phosphonate 10b. Following the above procedure with precursor 9b gave a colorless oil with analogous spectral properties but with [α]²⁰_{D}=−5.27 (c=0.22, MeOH).

Diethyl [1,1-Difluoro-3 (S), 4-Bis(oleoyl)butyl]phosphonate 11a. To a solution of diol (35 mg, 0.134 mmol) and oleic acid (91 mg, 0.322 mmol) in dry CH₂Cl₂ (1 mL) was added a solution of DCC (0.347 mmol) and DMAP (0.347 mmol) in dry CH₂Cl₂ (1 mL) at rt. The solution was stirred for 16 h at rt, filtered, concentrated in vacuo, and the residue was purified on silica gel (n-hexane/ethyl acetate=3:1, R_{f}=0.33) to ester (77 mg, 0.098 mmol, 73%) as a colorless oil. ¹H NMR (CDCl₃): 5.48 (m, 1H), 5.31 (m, 4H), 4.30-4.20 (m, 5H), 4.04 (dd, J=11.6, 5.6 Hz, 1H), 2.38 (m, 2H), 2.27 (m, 4H), 1.98 (m, 8H), 1.56 (m, 4H), 1.34 (t, J=8.0 Hz, 6H), 1.21 (m, 40H), 0.84 (t, J=6.8 Hz, 3H). ¹³C NMR (CDCl₃): 173.17 (s), 172.47 (s), 129.94 (s), 129.66 (s), 64.98 (m), 64.72 (dd, J=6.94, 6.13 Hz), 64.53 (s), 34.93 (td, J=19.91, 15.38 Hz), 34.18 (s), 33.97 (s), 31.85 (s), 29.71 (s), 29.67 (s), 29.47 (s), 29.27 (s), 29.14 (s), 29.07 (s), 29.00 (s), 27.16 (s), 27.13 (s), 24.79 (s), 24.71 (s), 22.63 (s), 16.32 (d, J=5.33 Hz), 14.05 (s). ¹⁹F NMR (CDCl₃): −111.63 (1F, dddd, J=260.41, 65.86, 23.71, 14.18 Hz), −112.40 (1F, ddt, J=260.41, 65.86, 23.71, 14.18 Hz). ³¹P NMR (CDCl₃): 7.18 (t, J=105.41 Hz). [α]²⁰_{D}=−1.02 (c=0.88, MeOH).

Diethyl [1,1-Difluoro-3 (R), 4-Bis(oleoyl)butyl]phosphonate 11b. ¹H NMR (CDCl₃): 5.48 (m, 1H), 5.31 (m, 4H), 4.31-4.21 (m, 5H), 4.04 (dd, J=11.6, 5.6 Hz, 1H), 2.38 (m, 2H), 2.28 (m, 4H), 1.98 (m, 8H), 1.58 (m, 4H), 1.35 (t, J=8.0 Hz, 6H), 1.21 (m, 40H), 0.84 (t, J=6.8 Hz, 3H). ¹³C NMR (CDCl₃): 173.17 (s), 172.48 (s), 129.95 (s), 129.67 (s), 65.00 (m), 64.71 (dd, J=6.94, 6.13 Hz), 64.54 (s), 34.48 (td, J=19.21, 16.09 Hz), 34.19 (s), 31.85 (s), 29.72 (s), 29.67 (s), 29.47 (s), 29.27 (s), 29.15 (s), 29.08 (s), 29.05 (s), 29.01 (s), 27.17 (s), 27.13 (s), 24.80 (s), 24.72 (s), 22.63 (s), 16.32 (d, J=5.43 Hz), 14.05 (s). ¹⁹F NMR (CDCl₃): −111.63 (1F, dddd, J=260.41, 65.86, 23.71, 14.18 Hz), −112.40 (1F, ddt, J=260.41, 65.86, 23.71, 14.18 Hz). ³¹P NMR (CDCl₃): 7.18 (t, J=105.41 Hz). MS (CI) m/z 791.4 (M⁺+1,100.00), 509.2 (M⁺—C₁₇H₃₃CO₂, 18.15). HRMS, M⁺, Found: 790.5684. Calcd for C₄₄H₈₁F₂O₇P, 790.5688. [α]²⁰_{D}=+1.47 (c=0.51, MeOH).

Sodium [1,1-difluoro-3 (R), 4-Bis(oleoyl)butyl]phosphonate 12b. Thoroughly dried precursor (35 mg, 0.035 mmol, 5 h under high vacuum) was dissolved in anhydrous methylene chloride (0.2 mL) at room temperature. Bromotrimethylsilane (46 μL, 0.35 mmol) was added with a dry syringe and stirred 4 h. TLC indicated that all of the reactant had disappeared, then the solvent removed under reduced pressure and dried under vacuum. The residue was dissolved in 95% methanol (1 mL) for 1 h and concentrated in vacuuo got colorless oil, which made a cloudy solution when dissolved in water. The water turned to clear after added 1-2 drops triethylamine (PH=7-8). This solution was absorbed to a sodium ion-exchange column (DOWEX 50WX8-200 resin, neutral Na⁺ form), and eluted with water. The fraction was lyophilized to give product (32 mg, 0.041 mmol, 93%). ¹H NMR (CD₃OD): 5.36 (m, 1H), 5.18 (m, 4H), 4.24 (d, J=11.2 Hz, 1H), 3.89 (m, 1H), 2.26 (m, 2H), 2.14 (m, 4H), 1.86 (m, 8H), 1.44 (m, 4H), 1.16-1.13 (m, 40H), 0.73 (m, 3H). ¹³C NMR (CD₃OD): 174.65 (s), 174.11 (s), 130.91 (s), 130.77 (s), 66.80 (m), 65.88 (s), 65.32 (m), 35.16 (s), 34.89 (s), 33.10 (s), 30.89 (s), 30.86 (s), 30.67 (s), 30.50 (s), 30.41 (s), 30.36 (s), 30.26 (s), 30.22 (s), 30.18 (s), 28.20 (s), 26.00 (s), 25.93 (s), 23.77 (s). ¹⁹F NMR (CD₃OD): −114.20 (m). ³¹P NMR (CD₃0D): 5.88 (t, J=252.80 Hz). [α]²⁰_{D}=+0.87 (c=0.58, MeOH).

Sodium [1,1-Difluoro-3 (S), 4-Bis(oleoyl)butyl]phosphonate 12a was obtained similarly, [α]²⁰_{D}=−0.52 (c=0.29, MeOH).

Diethyl [1,1-Difluoro-3 (S)-[(S)-α-methoxy-α-(trifluoromethyl)phenylacetyl]4-(oleoyl)butyl]phosphonate 13a. A solution of alcohol 9a (8 mg, 0.015 mmol) and (R)-α-methoxy-α-trifluoromethyl-phenylacetic acid chloride (15 mg, 0.061 mmol) in pyridine (1 mL) was stirred for 20 at rt The mixture was diluted with CH₂Cl₂ (10 mL), washed with aq. NaHCO₃ (3 mL), dried, filtered, and concentrated in vacuo. Flashed chromatography on silicon gel gave the corresponding MTPA ester as colorless oil (10 mg, 0.0135 mmol, 89%, HE:AE/2:1, R_{f}=0.27). ¹H NMR (CDCl₃): 7.52-7.49 (m, 2H), 7.39-7.35 (m, 3H), 5.76-5.71 (m, 1H), 5.34-5.31 (m, 2H), 4.35 (dd, J=12.0, 3.6 Hz, 1H), 4.29-4.23 (m, 4H), 4.03 (dd, J=12.0, 5.6 Hz, 1H), 3.53 (s, 3H), 2.56-2.41 (m, 2H), 2.18 (t, J=7.6 Hz, 2H), 1.98 (m, 4H), 1.52 (m, 2H), 1.38 (t, J=6.8 Hz, 6H), 1.25 (m, 20H), 0.86 (t, J=6.8 Hz, 3H). ¹³C NMR (CDCl₃): 173.01 (s), 165.66 (s), 131.98 (s), 130.02 (s), 129.72 (s), 129.61 (s), 128.36 (s), 127.34 (s), 67.58 (m), 64.91 (d, J=6.13 Hz), 64.14 (s), 55.49 (s), 34.98 (td, J=20.71, 15.38 Hz), 33.78 (s), 31.89 (s), 29.76 (s), 29.70 (s), 29.52 (s), 29.31 (s), 29.15 (s), 29.06 (s), 27.22 (s), 27.17 (s), 24.63 (s), 22.67 (s), 16.35 (d, J=5.33 Hz), 14.09 (s). ¹⁹F NMR (CDCl₃): −72.07 (s), −111.84 (1F, dtd, J=105.37, 22.58, 15.43 Hz), −112.11 (1F, ddt, J=105.37, 22.58, 15.43 Hz). ³¹P NMR (CDCl₃): 6.92 (t J=104.28 Hz).

Diethyl [1,1-Difluoro-3 (R)-[(S)-α-methoxy-α-(trifluoromethyl)phenylacetyl]4-(oleoyl)butyl]phosphonate 13b. A solution of alcohol 9b (18 mg, 0.034 mmol) and (R)-α-methoxy-α-trifluoromethyl-phenylacetic acid chloride (35 mg, 0.137 mmol) in pyridine (2 mL) was stirred for 20 at rt. The mixture was diluted with CH₂Cl₂ (20 mL), washed with aq. NaHCO₃ (5 mL), dried, filtered, and concentrated in vacuo. Flashed chromatography on silicon gel gave the corresponding MTPA ester as colorless oil (19 mg, 0.0256 mmol, 75%, HE:AE/2:1, R_{f}=0.26). ¹H NMR (CDCl₃): 7.53-7.51 (m, 2H), 7.38-7.34 (m, 3H), 5.81-5.75 (m, 1H), 5.33-5.29 (m, 2H), 4.44 (dd, J=12.4, 3.2 Hz, 1H), 4.26-4.18 (m, 4H), 4.09 (dd, J=12.0, 7.2 Hz, 1H), 3.53 (s, 3H), 2.47-2.27 (m, 2H), 2.56 (t, J=7.6 Hz, 2H), 1.98 (m, 4H), 1.55 (m, 2H), 1.36 (t, J=6.8 Hz, 6H), 1.26 (m, 20H), 0.85 (t, J=6.8 Hz, 3H). ¹³C NMR (CDCl₃): 173.01 (s), 165.48 (s), 131.88 (s), 129.99 (s), 129.70 (s), 129.61 (s), 128.32 (s), 127.34 (s), 67.58 (m), 64.86 (d, J=6.83 Hz), 64.35 (s), 55.37 (d, J=1.51 Hz), 34.80 (td, J=20.71, 15.38 Hz), 33.87 (s), 31.88 (s), 29.74 (s), 29.67 (s), 29.50 (s), 29.30 (s), 29.12 (s), 29.06 (s), 27.19 (s), 27.15 (s), 24.66 (s), 22.66 (s), 16.32 (d, J=5.33 Hz), 14.08 (s). ¹⁹F NMR (CDCl₃): −72.07 (s), −112.10 (1F, dtd, J=103.86, 22.20, 16.93 Hz), −112.38 (1F, ddt, J=103.86, 22.20, 16.93 Hz). ³¹P NMR (CDCl₃): 6.81 (t, J=104.28 Hz).

II. Synthesis of Difluoro Analogs of LPA

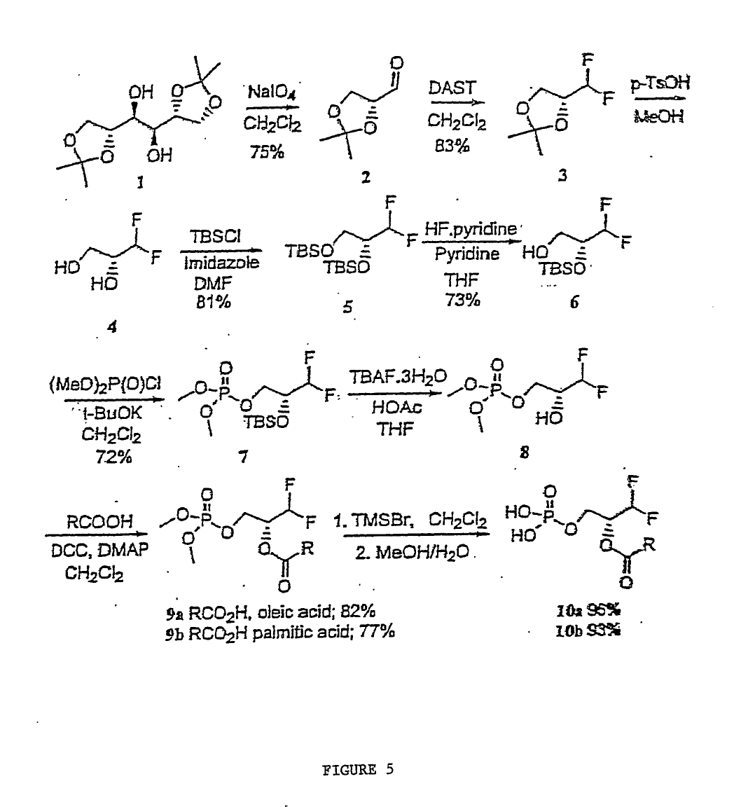

Another approach to the synthesis of difluoromethylene analogs of LPA is depicted in FIG. 5. Synthesis of the target LPA analogues 10a and 10b (FIG. 5) involved non-reductive deprotection of the penultimate dimethyl phosphates 9 with trimethylsilane bromide to permit incorporation of unsaturated acyl chains. The key step for the synthesis was the introduction of the difluoromethyl group by the 1,1-difluorination of a C-1 aldehyde. Thus, commercially-available D-mannitol 1,2:5,6-bis-acetonide was oxidatively cleaved with NaIO₄ to afford the acetonide-protected D-glyceraldehyde 2.10 Addition of (diethylamino)sulfur trifluoride (DAST) to a solution of the aldehyde 2 in CH₂Cl₂ afforded the difluorinated compounds in high yield after purification by distillation under reduced pressure.

Next, acidic cleavage of the acetonide-protecting group provided the diol intermediate 4. The crude diol obtained after removal of the acetonide was immediately converted to the bis-silyl ether 5, and the more labile TBDMS ether of the primary alcohol was cleaved selectively by treatment with a solution of pyridinium hydrofluoride in a mixture of pyridine and THF at rt. Initial attempts to obtain the primary alcohol 6 from bis-TBDMS ether 5, utilizing 4.0 eq. of pyridinium hydrofluoride resulted in disappointing yields (17%) after 48 h at rt. However, increasing to 6.0 equiv. gave the primary alcohol in good yield (73%) after 20 h at rt. The primary alcohol 6 was then phosphorylated with dimethylphosphoryl chloride in the presence of t-BuOK to give good yield of phosphate 7. The 2-TBDMS ether was further deprotected with tetra(n-butyl)ammonium fluoride (TBAF) in THF to give alcohol 8 in 72% yield; neutralization of TBAF with acetic acid permitted desilyation of the secondary alcohol without the migration of phosphate. DCC-promoted esterification of alcohol 8 with oleic acid or palmitic acid provided good yield of esters 9a and 9b, respectively. Importantly, the introduction of the acyl groups at this stage circumvents problems with acyl group migration during other synthetic operations. Finally, treatment of protected phosphates 9 with bromotrimethylsilane and subsequent addition of 5% aq. methanol provided the desired difluorinated LPA analogues 10 in essentially quantitative yield.

General procedures. Chemicals were obtained from Aldrich and Acros and used without prior purification. Solvents were reagent-grade and distilled before use: THF was distilled from sodium wire, and CH₂Cl₂ was distilled from CaH₂. Reactions were performed under an inert atmosphere (N₂ or Ar) unless otherwise indicated. NMR spectra were recorded at 25° C. at 400 MHz (1H), 101 MHz (¹³C), 162 MHz (³¹P) and 376 MHz (¹⁹F). Chemical shifts are given in ppm relative to tetramethylsilane as the internal standard for ¹H and ¹³C spectra (δ=0.00); external standards were used for ³¹P (85% H₃PO₄, δ=0.00) and ¹⁹F (CFCl₃, δ=0.00).

(R)-Glyceraldehyde acetonide (2) was prepared from D-mannitol-1,2:5,6-bis-acetonide as described 10 to give aldehyde 2 as a clear liquid: [α]²⁰_{D}: +64.4 (lit.19 [α]²⁰_{D}+64.9).

(2R)-3,3-Difluoro-1,2-propanediol 1,2-acetonide (3). To a well-stirred solution of 8.10 g (62.3 mmol) of aldehyde 2 in dry CH₂Cl₂ (100 mL) was slowly added 10.2 mL (74.8 mmol) of DAST. After stirring 24 h at rt, the reaction mixture was quenched with 10% NaHCO₃ solution (80 mL). The aqueous layer was extracted with CH₂Cl₂ (2×100 mL) and the combined organics were dried (Na₂SO₄). The solvent was removed by fractional distillation until the head temperature reached 40° C. The residue was then distilled at reduced pressure (ca. 24 mm Hg), collecting the fraction distilling at 65-66° C. to give 6.5 g (51.2 mmol, 83%) of difluoride 3 as a clear liquid. ¹H NMR (CDCl3): δ 5.68 (td, J=56.0, 4.8 Hz, 1H), 4.23 (m, 1H), 4.10 (m, 2H), 1.45 (s, 3H), 1.37 (s, 3H). ¹³C NMR (CDCl3): δ 114.83 (t, J=243.9 Hz), 111.19 (s), 74.83 (t, J=27.6 Hz), 64.19 (dd, J=5.3, 2.0 Hz), 26.50 (s), 25.11 (s). 19F NMR (CDCl3): δ −127.02 (1F, ddd, 2JFF=292.0, 2JFH=54.0, 3JFH=10.5 Hz), −129.82 (1F, ddd, 2JFF=292.0, 2JFH=54.0, 3JFH=10.5 Hz). MS (CI) m/z 153.0 (M⁺+1, 100.00), 137.0 (M⁺—CH₃, 6.56). HRMS, M⁺+1, Found: 153.0739. Calcd for C₆H₁₁O₂F₂, 153.0727. [α]²⁰_{D}: −3.1 (1.09, MeOH).

(2R)-3,3-Difluoro-1,2-di{[1(t-butyl)-1,1-dimethylsilyl]oxyl}-propane (5). To a solution of acetonide 3 (2.20 g, 14.47 mmol) in MeOH (30 mL) was added pTsOH (0.412 g, 2.17 mmol, 0.15 eq.) and the solution was stirred for 24 h at rt. After addition of NEt₃ (1 mL), the solvent was removed under reduced pressure. Next, crude diol 4 was dissolved in anhydrous DMF (16 mL) and stirred with imidazole (2.96 g, 43.41 mmol, 2.9 eq.) and t-butyldimethylsilyl chloride (TBSCl) (6.11 g, 40.52 mmol, 2.8 eq.) for 24 h at rt. The solution was diluted with water (60 mL) and ethyl acetate (100 mL), and the aqueous layer was separated and extracted with ethyl acetate (3×80 mL). The combined organic layers were dried (Na₂SO₄), concentrated in vacuo, and the residue was purified on silica gel (n-hexane-ethyl acetate 60:1, R_{f}=0.36) to afford bis-TBDMS ether 5 as a colorless liquid 3.97 g (11.68 mmol, 81%). ¹H NMR (CDCl₃): δ 5.67 (td, J=55.6, 4.0 Hz, 1H), 3.72 (m, 2H), 3.62 (m, 1H), 0.84 (s, 9H), 0.83 (s, 9H), 0.04 (s, 3H), 0.03 (s, 3H), 0.003 (s, 3H), 0.000 (s, 3H). ¹³C NMR (CDCl₃): δ 120.79 (t, J=243.5 Hz), 78.26 (dd, J=23.7, 21.4 Hz), 68.83 (t, J=4.5 Hz), 31.40 (s), 31.24 (s), 23.86 (s), 23.73 (s), 0.76 (s), 0.58 (s), 0.03 (s), 0.00 (s). ¹⁹F NMR (CDCl₃): δ −130.58 (1F, ddd, J (as above)=284.1, 55.3, 5.3 Hz), −134.05 (1F, ddd, J=284.1, 55.3, 5.3 Hz). MS (CI) m/z 314.2 (M⁺+1, 100.00), 283.1 (M+—C4H9, 10.42). HRMS, M⁺+1, Found: 341.2134. Calcd for C₁₅H₃₅O₂F₂Si₂, 341.2143. [α]²⁰_{D}: −10.1 (0.61, MeOH).

(2R)-3,3-Difluoro-2-di[[1-(t-butyl)-1,1-dimethylsilyl]oxyl]-1-propanol (6). The HF-pyridine complex (70%, 30 mmol fluoride) was added to a mixture of pyridine (2.62 mL), and then a solution of bis-ether 5 (1.70 g, 5.00 mmol) in THF (25 mL) was added. The reaction mixture was stirred for 20 h at rt. After completion of the reaction (monitored by TLC), the solution was diluted with ethyl acetate (100 mL), washed with 0.5 M HCl (2×20 mL) and then with satd. CuSO₄ solution (20 mL), and dried (Na₂SO₄). After concentration in vacuo, the residue was purified on silica gel (n-hexane-ethyl acetate 5:1, R_{f}=0.31) to afford 0.82 g of mono-ether 6 as a colorless liquid (3.63 mmol, 73%). ¹H NMR (CDCl₃): δ 5.58 (td, J=53.6, 6.0 Hz, 1H), 3.68 (m, 2H), 3.59 (m, 1H), 1.79 (br, 1H), 0.79 (s, 9H), 0.00 (s, 6H). ¹³C NMR (CDCl₃): δ 120.05 (t, J=234.5 Hz), 77.37 (dd, J=27.6, 22.3 Hz), 67.21 (dd, J=6.5, 3.0 Hz), 30.62 (s), 23.06 (s), 0.11 (s), 0.00 (s). ¹⁹F NMR (CDCl₃): δ −128.55 (1F, ddd, J=289.4, 55.3,6.4 Hz), −130.25 (1F, ddd, J=289.4, 55.3, 6.4 Hz). MS (CI) m/z 227.1 (M⁺1, 100.00), 169.0 (M⁺—C4H9, 8.11). HRMS, M⁺+1, Found: 227.1264. Calcd for C₉H₂₁O₂F₂Si, 227.1279. [α]²⁰_{D}: −11.3 (0.79, MeOH).

(2R)-3,3-Difluoro-2-di[[1-(t-butyl)-1,1-dimethylsilyl]oxyl]-1-phospho-propane dimethyl ester (7). To a stirred solution of 128 mg (0.566 mmol) of ether 6 and dimethyl chlorophosphate (98 mg, 0.679 mmol, 1.2 eq.) in CH₂Cl₂ (10 mL) at 0° C. was added t-BuOK (89 mg, 0.792 mmol, 1.4 eq.). The mixture was stirred 2 h at rt and the reaction was complete as determined by TLC. The reaction was quenched by addition of satd. aq. NH₄Cl (5 mL), the mixture was stirred 10 min, and the aqueous phase was extracted with CH₂Cl₂ (3×5 mL). The organics were dried (Na₂SO₄), concentrated, and purified on silica gel (n-hexane-ethyl acetate 3:2, R_{f}=0.41) to afford 136 mg of phosphotriester 7 as a colorless liquid (0.407 mmol, 72%). ¹H NMR (CDCl₃): δ 5.88 (td, J=53.2, 3.2 Hz, 1H), 4.38 (m, 2H), 3.83 (m, 1H), 3.73 (d, J=0.8 Hz, 3H), 3.70 (d, J=0.8 Hz, 3H), 0.81 (s, 9H), 0.002 (s, 3H), 0.000 (s, 3H). ¹³C NMR (CDCl₃): δ 118.80 (td, J=234.2, 6.9 Hz), 81.75 (t, J=22.2 Hz), 66.65 (dd, J=8.5, 3.1 Hz), 60.21 (t, J=6.54 Hz), 31.35 (s), 23.85 (s), 0.00 (s), −0.03 (s). ¹⁹F NMR (CDCl₃): δ −131.75 (1F, ddd, J=292.4, 54.6, 7.9 Hz), −134.1 (1F, ddd, J=292.4, 54.6, 7.9 Hz). ³¹P NMR (CDCl₃): δ 1.467 (s). MS (CI) m/z 335.0 (M⁺+1, 100.00), 276.9 (M⁺—C4H10, 13.15). HRMS, M⁺1, Found: 335.1258. Calcd for C₁₁H₂₆F₂O₂PSi, 335.1255. [α]₂₀_{D}: −75.7 (0.504, MeOH).

(2R)-3,3-Difluoro-2-oleoyl-1-phospho-propane dimethyl ester (9a). A solution of TBDMS ether 7 (59 mg, 0.178 mmol) in THF (5 mL) was treated successively with acetic acid (41 μL, 0.706 mmol) and tetrabutylammoniumfluoride trihydrate (223 mg, 0.706 mmol) at rt. After stirring for 4 h, the reaction was complete (TLC), and the solvent removed in vacuo and the crude product was purified only by passing through a short silica gel bed (ethyl acetate , R_{f}=0.48) and concentrated in vacuo to afford the alcohol 8 as a colorless liquid. To the crude alcohol 8 was added 55 mg (62 μL, 0.194 mmol) of oleic acid in dry CH₂Cl₂ (2 mL) followed by dropwise addition of a solution of DCC (55 mg, 0.266 mmol) and DMAP (13 mg, 0.106 mmol) in dry CH₂Cl₂ (3 mL). The solution was stirred for 16 h at rt, filtered, concentrated in vacuo, and purified on silica gel (n-hexane-ethyl acetate 1:1, R_{f}=0.26) to afford 71 mg of oleate 9a as a waxy solid (0.146 mmol, 82%). ¹H NMR (CDCl₃): δ 5.86 (td, J=54.8, 4.0 Hz, 1H), 5.28 (m, 2H), 5.15 (m, 1H), 4.20 (m, 2H), 3.73 (d, J=4.4 Hz, 3H), 3.70 (d, J=4.4 Hz, 3H), 2.34 (t, J=7.6 Hz, 2H), 1.93 (m, 4H), 1.58 (m, 2H), 1.22 (m, 20H), 0.81 (t, J=6.4 Hz, 3H). ¹³C NMR (CDCl₃): δ 172.52 (s), 130.25 (s), 129.90 (s), 112.72 (t, J=244.6 Hz), 70.04 (td, J=25.24, 7.64 Hz), 63.91 (d, J=4.6 Hz), 54.76 (d, J=6.1 Hz), 34.18 (s ), 34.09 (s), 32.11 (s), 29.97 (s), 29.88 (s), 29.73 (s), 29.53 (s), 29.33 (s), 29.27 (s), 29.18 (s), 27.43 (s), 27.36 (s), 25.16 (s), 24.92 (s), 22.88(s), 14.31(s). ¹⁹F NMR (CDCl₃): δ −130.101 (1F, ddd, J=294.7, 53.8, 10.5 Hz), −131.0 (1F, ddd, J=294.7, 53.8, 10.5 Hz). ³¹P NMR (CDCl₃): δ 3 2.111 (s). MS (CI) m/z 485.3 (M⁺+1, 64.53), 359.2 (M³⁰ —C₂H₆PO₄, 100.00). HRMS, M⁺+1, Found: 485.2867. Calcd for C₂₃H₄₄F₂O₆P, 485.2844. [α]²⁰_{D}: −8.6 (1.08, MeOH).

(2R)-3,3-Difluoro-2-oleoyl-1-phospho-propane (10a). An aliquot of protected ester 9a (55 mg, 0.114 mmol) was thoroughly dried (5 h, 1 μm Hg), dissolved in dry CH₂Cl₂ (2 mL) at rt, and then bromotrimethylsilane (53 μL, 0.398 mmol) was added dropwise with a dry syringe and the mixture was stirred for 4 h at rt. When TLC indicated that all of the reactant had disappeared, solvents were removed in vacuo, the residue was dissolved in 95% methanol (1 mL) for 1 h, and then reconcentrated in vacuo to give 50 mg of LPA 2-oleate analogue 10a as a colorless oil (0.110 mmol, 96%) that was homogeneous by TLC: CH₂Cl₂/CH₃OH/H₂O, 20:10:1, R_{f}=0.58. ¹H NMR (CD₃OD): δ 6.03 (t, J=54.4 Hz, 1H), 5.53 (m, 2H), 5.24 (m, 1H), 4.18 (m, 2H), 2.41 (t, J=7.2 Hz, 2H), 2.02 (m, 4H), 1.63 (m, 2H), 1.30 (m, 20H), 0.89 (t, J=6.4 Hz, 3H). ¹³C NMR (CD₃OD): δ 173.70 (s), 130.88 (s), 130.78 (s), 114.43 (t, J=242.4 Hz), 71.22 (td, J=23.73, 8.45 Hz), 63.89 (d, J=4.6 Hz), 34.67 (s), 33.06 (s), 30.84 (s), 30.78 (s), 30.61 (s), 30.44 (s), 30.34 (s), 30.26 (s), 30.16 (s), 30.04 (s), 28.12 (s), 25.84 (s), 23.73 (s), 14.15 (s). ¹⁹F NMR (CD₃OD): δ −130.10 (1F, ddd, J=295.8, 55.3, 9.4 Hz), −131.7 (1F, ddd, J=295.8, 55.3, 9.4 Hz). ³¹P NMR (CDCl₃): δ 0.742 (s). MS (CI) m/z 457.2 (M⁺+1, 13.75), 377.2 (M++2−H2PO3, 100.00). HRMS, M⁺+1, Found: 457.2535. Calcd for C₂₁H₄₀F₂O₆P, 457.2531. [α]²⁰_{D}: −9.3 (1.02, MeOH).

(2R)-3,3-Difluoro-2-palmitoyl-1-phospho-propane dimethyl ester (9b). A solution of TBDMS ether 7 (59 mg, 0.178 mmol) in THF (5 mL) was treated successively with acetic acid (41 μL, 0.706 mmol) and tetrabutylammoniumfluoride trihydrate (223 mg, 0.706 mmol) and processed as described for 9a to give crude alcohol 8. The crude alcohol was directly esterified with 50 mg (0.194 mmol) of palmitic acid in dry CH₂Cl₂ (2 mL) at rt by dropwise addition of a solution of DCC (55 mg, 0.266 mmol) and DMAP (13 mg, 0.106 mmol) in dry CH₂Cl₂ (3 mL). The solution was stirred for 16 h at rt, filtered, concentrated in vacuo, and the residue was purified on silica gel (n-hexane/ethyl acetate 1:1, R_{f}=0.36) to afford 62 mg of ester 9b a waxy solid (0.136 mmol, 77%). ¹H NMR (CD₃OD): δ 6.05 (td, J=54.8, 4.4 Hz, 1H), 5.30 (m, 1H), 4.29 (m, 2H), 3.80 (d, J=5.2 Hz, 3H), 3.77 (d, J=4.8 Hz, 3H), 2.42 (t, J=7.6 Hz, 2H), 1.64 (m, 2H), 1.28 (m, 24H), 0.89 (t, J=6.8 Hz, 3H). ¹³C NMR (CD₃OD): δ 173.59 (s), 114.34 (t, J=244.0 Hz), 71.11 (td, J=25.34, 6.94 Hz), 65.39 (d, J=5.3 Hz), 54.42 (d, J=6.1 Hz), 34.76 (s), 34.65 (s), 33.08 (s), 30.78 (s), 30.69 (s), 30.57 (s), 30.48 (s), 30.37 (s), 30.04 (s), 26.76 (s), 26.05 (s), 25.86 (s), 23.73 (s), 14.44 (s). ¹⁹F NMR (CD₃OD): δ −131.7 (1F, dt, J=55.3, 10.5 Hz), −131.9 (1F, dt, J=55.3, 10.5 Hz). ¹⁹F NMR (CDCl₃): δ −130.1 (1F, ddd, J=296.2, 55.3, 12.0 Hz), −131.0 (1F, ddd, J=296.2, 55.3, 12.0 Hz). ³¹P NMR (CD₃OD): δ 1.816 (s). MS (CI) m/z 459.3 (M⁺+1, 83.09), 333.2 (M⁺—C₂H₆PO₄, 100.00). HRMS, M⁺+1, Found: 459.2708. Calcd for C₂₁H₄₂F₂O₆P, 4592687. [α]²⁰_{D}: −10.3 (0.80, MeOH).

(2R)-3,3-Difluoro-2-oleoyl-1-phospho-propane (10b). As described for 10a, thoroughly dried ester 9b (38 mg, 0.083 mmol) was dissolved in dry CH₂Cl₂ (1 mL) and deprotected with bromotrimethylsilane (38 μL, 0.290 mmol). The crude product was dissolved in 95% methanol (1 mL) for 1 h and reconcentrated and thoroughly dried in vacuo to give 33 mg of LPA palmitate analogue 10b (0.077 mmol, 93%). ¹H NMR (CD₃OD): δ 5.81 (td, J=55.2, 4.4 Hz, 1H), 5.03 (m, 1H), 3.96 (m, 2H), 2.20 (t, J=6.8 Hz, 2H), 1.41 (m, 2H), 1.07 (s, 24H), 0.68 (t, J=6.8 Hz, 3H). ¹³C NMR (CD₃OD): δ 173.72 (s), 114.43 (t, J=242.3 Hz), 71.22 (td, J=23.73, 8.45 Hz), 63.92 (d, J=4.6 Hz), 34.68 (s), 33.08 (s), 30.79 (s), 30.77 (s), 30.72 (s), 30.58 (s), 30.48 (s), 30.39 (s), 30.07 (s), 25.86 (s), 23.74 (s), 14.46 (s). ¹⁹F NMR (CD₃OD): −132.08 (1F, ddd, J=295.4, 54.2, 9.4 Hz), −132.7 (1F, ddd, J=295.4, 54.2, 9.4 Hz). ³¹P NMR (CD₃OD): 0.709 (s). MS (CI) m/z 431.1 (M⁺+1, 3.39), 333.1 (M⁺—H2PO4, 100.00). HRMS, M⁺+1, Found: 431.2369. Calcd for C₁₉H₃₈F₂O₆P, 431.2375. [α]²⁰_{D}: −2.1 (0.90, MeOH). (2R-3,3-Difluoro-2-O-[(S)-α-methoxy-α-(trifluoromethyl)phenylacetyl]-1-phospho-propane dimethyl ester (11). A solution of alcohol 8 and (R)-methoxy-(trifluoromethyl)phenylacetic acid chloride in pyridine was stirred for 20 h at rt. The mixture was diluted with CH₂Cl₂, washed with aq. NaHCO₃, dried, filtered, and concentrated. Flash chromatography on silica gel gave the corresponding MTPA ester as colorless oil. ¹H NMR (CDCl₃): δ 7.52 (m, 2H), 7.40 (m, 3H), 5.87 (td, J=54.4, 4.0 Hz, 1H), 5.47 (m, 1H), 4.40 (m, 1H), 4.28 (m, 1H), 3.72 (d, J=8.0 Hz, 3H), 3.75 (d, J=8.0 Hz, 3H), 3.55 (m, 3H). ¹⁹F NMR (CDCl₃): −72.36 (s), −129.37 (1F, ddd, J=296.2, 55.3, 11.0 Hz), −130.27 (1F, ddd, J=296.2, 55.3, 11.0 Hz); −72.17 (1.59), −72.36 (98.41), >97% ee. ³¹P NMR (CDCl₃): δ 1.728 (s).

III. Synthesis of Hydroxyethoxy Substituted Analogs of LPA

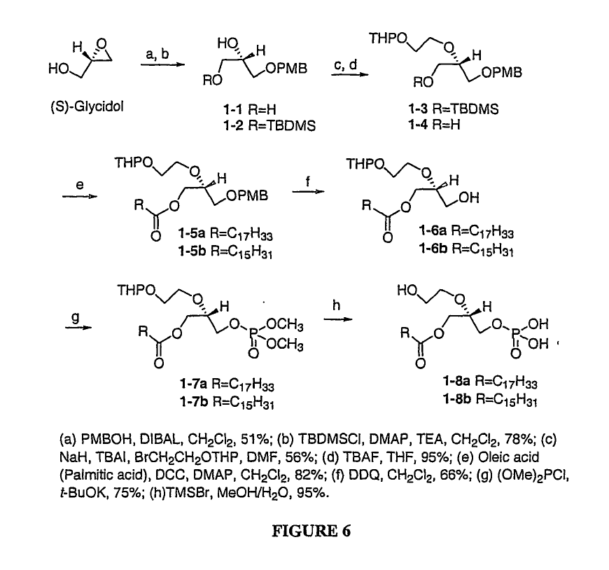

In the routes leading to syn-1 HE-LPA analogs (FIG. 6), the regiospecific and stereospecific ring opening of (S)-glycidol with 4-methoxybenzyl (PMB) alcohol by diisobutylaluminium hydride (DIBAL), generated the PMB protected glycerol (1-1). Using 4-(dimethylamino) pyridine (DMAP) as the catalyst, the primary alcohol of the diol was selectively silylated over the secondary alcohol by t-butyldimethylsilyl chloride in 78% yield. Initial attempts to obtain (1-3) from the secondary alcohol (1-2), using 2(2-bromoethoxy) tetrahydro-2-H-pyran in the presence of NaH in anhydrous DMF, resulted in no product after 48 h at room temperarture. However adding tetrabutylammonium iodide (TBAI) into the reaction gave the alkylated product in 56% yield after 18 h at room temperarture. Then the 1-TBDMS ether was deprotected with tetra(n-butyl)ammonium fluoride (TBAF) in THF to give alcohol (1-4), which was esterified with oleic acid or palmitic acid using DCC and DMAP to produce good yields of esters (1-5a) and (1-5b), respectively. Oxidative removal of the PMB groups with 2,3-dichloro-5,6-dicyanobenzoquinone (DDQ) produced corresponding alcohols (1-6a) and (1-6b). They were then phosphorylated with dimethyl chlorophosphate in the presence of t-BuOK to give good yields of phosphates (1-7a) and (1-7b). The non-reductive deprotection of dimethyl phosphates with bromotrimethylsilane was compatible with the unsaturated acyl chains. The trace of acid generated during workup (adding MeOH/H₂O) resulted in elimination of tetrahydropyranyl groups (THP) and generation of our target compounds (1-8a) and (1-8b).

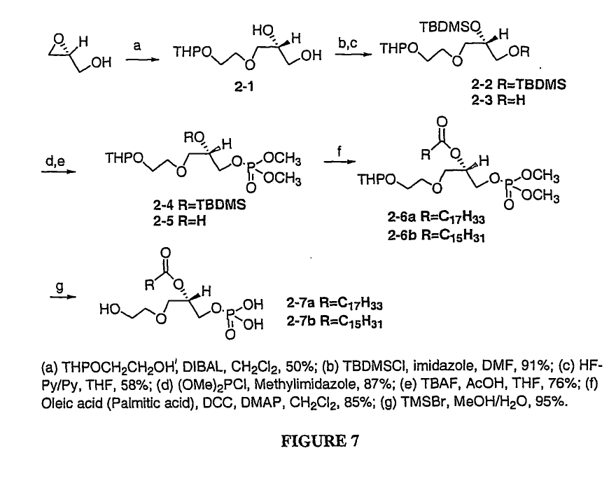

The strategies for the synthesis of non-migrating sn-2 HE-LPA analogs were similar to those used for the preparation of sn-1 HE-LPA (FIG. 7). In order to get (2S) enantiomer of the sn-2 HE-LPA analogs, (R)-Glycidol was used. After the regiospecific and stereospecific ring opening of Glycidol and TBDMS protection of the diol, the selective deprotection of bis-TBDMS ether (2-2) utilizing 6.0 eq. of pyridinium hydrofluoride (HF-Py/Py), resulted in 58% yield after 18 h at room temperarture. The amount of HF-Py was crucial to the reaction since more would cause deprotection of both TBDMS groups and less amount would lead to low yields. Interestingly, phosphorylation of (2-3) using mtheylimidazole instead of t-BuOK increased the yield from 10% to 87%. The 2-TBDMS ether was further deprotected with TBAF.3H₂O in THF to give alcohol (2-5); neutralization of TBAF with acetic acid allowed the desilyation of the secondary alcohol without the migration of phosphate. After DCC-promoted esterification and TMSBr deprotection, syn-2 LPA analogs (2-7a) and (2-7b) were obtained in good yields.

The enantiomeric purity of (1-2) and (2-5) was determined by Mosher's ester method, and optical purities were measured by integration of the ¹H-NMR.

General Procedures. Chemicals were purchased from Aldrich and Acros Chemical Corporation and used without prior purification. Solvents were reagent-grade and distilled before use: CH₂Cl₂ was distilled from CaH₂ and THF was distilled from sodium wire. TLC: precoated silica) gel aluminum sheets (EM SCIENCE silica gel 60F₂₅₄). Flash Chromatography (FC): Silica gel Whatman 230˜400 mesh astm. NMR spectra were recorded on a Varian INOVA 400 at 400 MHz (¹H), 101 MHZ (¹³C), 162 MHz (³¹P) at 25° C. Chemical shifts are given in ppm with TMS as internal standard (δ=0.00); ³¹p, 85% H₃PO₄ (δ=0.00).

3-O-Methoxybenzyl-sn-glycerol (1-1). To a solution of PMBOH (9.8 g, 70 mmol) in 25ml anhydrous CH₂Cl₂in an ice bath, 1.0M DIBAL-H in Hexane (30 mL) was added. The reaction mixture was warmed to rt and stirred for 0.5 h. (S)-Glycidol (2 mL, 30 mmol) was added to the reaction mixture which was then stirred at rt for 70 h. Sodium potassium tartrate (6.3 g, 30 mmol) in a minimum amount of water was then added to the mixture and stirring continued for 0.5 h. The solvent was evaporated and the mixture was extracted with ethyl acetate, washed with water, dried over sodium sulfate, and concentrated. The crude product was purified by flash chromatography (EtOAc) to afford colorless oil 3.3 g (51%). R_{f}0.28 (EtOAc); ¹H-NMR (CDCl₃) δ 3.517 (m, 2H), 3.599 (dd, 1H, J=11.2, 5.4 Hz), 3.5678 (dd, 1H, J=11.2, 3.4 Hz), 3.798 (s, 3H), 3.862 (m, 1H), 4.472 (s, 2H), 6.878 (dd, J=8.4, 2.0 Hz), 7.242 (dd, J=8.0, 2.4 Hz); ¹³C-NMR, δ 55.253, 64.054, 70.574, 71.474, 73.220, 113.875, 129.440, 129.722, 159.372; MS (FAB) m/z 235 (M⁺+Na, 24). HRMS, M⁺+Na, Found:235.0939, Calcd for C₁₁H₁₆O₄Na, 235.0946.

3-O-tert-butyl-dimethysily-1-O-Methoxybenzyl-sn-glycerol (1-2). A mixture of 1-1 (950 mg, 4.48 mmol), tert-butyldimethylsilyl chloride (810 mg, 5.4 mmol), TEA (546 mg, 5.4 mmol) and DMAP (55 mg, 0.448 mmol) in anhydrous CH₂Cl₂(15 mL) under an argon atmosphere was stirred at rt for 18 h. The reaction mixture was washed with NaCl saturated solution, dried over Na₂SO₄, and concentrated. FC (EtOAc/Hexane, 1/4, v/v) gave 1-2 as a colorless oil 980 mg (78%). R_{f}0.31 (EtOAc/Hexane 1/4); ¹H-NMR (CDCl₃, 400 MHz) δ 0.0 (s, 6H), 0.828 (s, 9H), 3.430 (m, 2H), 3.579 (m, 2H), 3.737 (s, 3H), 3.782 (m, 1H), 4.417 (s, 2H), 6.815 (dd, J=8.8, 2.0 Hz), 7.192 (dd, J=8.8, 2.0 Hz); ¹³C-NMR, δ −5.457, 18.237, 25.825, 55.208, 63.993, 70.628, 70.643, 73.045, 113.761, 129.333, 130.126, 159.228; MS (FAB) m/z 325 (M⁺+H, 7). HRMS, M⁺+H, Found: 325.1831, Calcd for C₁₇H₂₉O₄Si, 325.1835.

3-O-tert-butyl-dimethysily-1-O-Methoxybenzyl-2-O-(tetrahydro-pyran-2-yloxy)ethyl-sn-glycerol (1-3). To a solution of 2 (900 mg, 2.76 mmol) in dry DMF (25 mL) was added 60% NaH in oil dispersion (375 mg, 9.4 mmol). The mixture was stirred at rt for 0.5 h. The bromide (1.25 ml, 8.28 mmol) and TBAI (1 g, 2.76 mmol) was added to the reaction. The mixture was stirred at rt for 18 h. After adding 5 ml H₂O, the solvent was evaporated. The mixture was extracted with EtOAc (20 mL×3). The extract was washed with NaCl saturated solution, dried over Na₂SO₄, and concentrated. FC (EtOAc/Hexane, 1/4, v/v) gave 1-3 as a colorless oil mg (56%). R_{f} 0.35 (EtOAc/Hexane 1/4); ¹H-NMR (CDCl₃) δ 0.004 (s, 6H), 0.841 (s, 9H), 1.513 (m, 4H), 1.718 (m, 2H), 3.450 (m, 2H), 3.531 (m, 2H), 3.624 (m, 2H), 3.724˜3.754 (m, 1H), 3.759 (s, 3H), 3.802 (m, 2H), 4.44 (d, 2H, J=2.4 Hz), 4.586 (t, 1H, J=3.6 Hz), 6.824 (dd, J=8.4, 1.6 Hz), 7.195(dd, J=8.4, 1.6 Hz); ¹³C-NMR, δ −5.423, −5.377, 18.264, 19.431, 25.455, 25.875, 30.572, 55.258, 62.083, 62.114, 62.579 (d, J=7.68 Hz), 66.956 (d, J=7.68 Hz), 69.809 (d, J=6.16 Hz), 80.149 (d, J=7.68 Hz), 98.856 (d, J=7.68 Hz), 113.704, 113.818, 129.215, 129.360, 130.558, 159.102; MS (FAB) m/z 477 (M⁺+Na, 17). HRMS, M⁺+Na, Found: 477.2629, Calcd for C₂₄H₄₂O₆NaSi, 477.2648.

3-O-Methoxybenzyl-2-O-(tetrahydro-pyran-2-yloxy)ethyl-sn-glycerol (1-4). To a solution of 1-3 (330 mg, 0.726 mmol) in THF (5 mL) was added 1M TBAF in THF (1.45 mL). The reaction mixture was stirred at rt for 3 h. The mixture was washed with NaCl saturated solution, dried over Na₂SO₄, and concentrated. FC (EtOAc/Hexane, 3/1, v/v) gave 1-4 as a colorless oil 241 mg (95%). R_{f}0.22 (EtOAc/Hexane 3/2); ¹H-NMR (CDCl₃) δ 1.550 (m, 4H), 1.762 (m, 2H), 2.5 (br, 1H), 3.474˜3.743 (m, 7H), 3.805 (s, 3H), 3.858 (m, 2H), 4.464 (s, 2H), 4.637 (m, 1H), 6.876 (dd, J=7.6, 2.0 Hz), 7.251 (dd, J=7.6, 2.0 Hz); ¹³C-NMR, 19.393 (d, J=3.13 Hz), 25.287, 30.466 (d, J=7.78 Hz), 55.243, 62.335 (d, J=4.65 Hz), 62.838 (d, J=12.32 Hz), 67.132 (d, J=18.48 Hz), 69.824, 69.9 (d, J=4.65 Hz), 73.118, 79.745, 99.013 (d, J=10 Hz), 113.78, 129.254, 129.383, 130.115, 159.224; MS (FAB) m/z 363 (M⁺+Na, 33). HRMS, M⁺+Na, Found: 363.1769, Calcd for C₁₈H₂₈O₆Na, 363.1784.

1-O-Methoxybenzyl-3-O-Oleoyl-2-O-(tetrahydro-pyran-2-yloxy)ethyl-sn-glycerol (1-5a). A solution of 1-4 (240 mg, 0.705 mmol), oleic acid (319 mg, 1.13 mmol), DCC (233 mg, 1.13 mmol), DMAP (40 mg, 0.141 mmol) in CH₂Cl₂ (10 ml) was stirred at rt for 18 h, filtered through Celite, and concentrated. FC (EtOAc/Hexane, 1/4, v/v) gave 1-5a as a colorless oil 350 mg (82%). R_{f}0.26 (EtOAc/Hexane 1/4) ¹H-NMR (CDCl₃). δ 0.874 (t, J=6.8 Hz, 3H), 1.275 (m, 20H), 1.4˜1.8 (m, 8H), 2.002 (m, 2H), 2.284 (t, J=7.6 Hz, 2H), 3.45˜3.85 (m, 7H), 3.796 (s, 3H), 4.2 (m, 2H), 4.472 (s, 2H), 4.619 (m, 1H), 5.336 (m, 2H), 6.854 (dd, J=8.8, 2.0 Hz), 7.237 (dd, J=8.8, 2.0 Hz); ¹³C-NMR; MS (FAB) m/z 627 (M⁺+Na, 43). HRMS, M⁺+Na, Found: 627.4203, Calcd for C₃₆H₆₀O₇Na, 627.4237.

3-O-Oleoyl-2-O-(tetrahydro-pyran-2-yloxy)ethyl-sn-glycerol (1-6a). A solution of 1-5a (340 mg, 0.562 mmol), DDQ (128 mg, 0.562 mmol) in wet CH₂Cl₂ (10 mL) was stirred at rt for 8 h. After filtration, the filtrate was washed with NaCl saturated solution, dried over Na₂SO₄, and concentrated. FC (EtOAc/Hexane, 2/3, v/v) gave 1-6a as a colorless oil 180 mg (66%). R_{f}0.36 (EtOAc/Hexane 1/1); ¹H-NMR (CDCl₃, 400 MHz), δ 0.877 (t, J=7.2 Hz, 3H), 1.273 (m, 20H), 1.52˜1.804 (m, 8H), 2.006 (m, 2H), 2.319 (t, J=7.2 Hz, 2H), 3.50˜3.76 (m, 6H), 3.92 (m, 3H), 4.13 (m, 2H), 4.65 (m, 1H), 5.34 (m, 2H); MS (FAB) m/z 507 (M⁺+Na, 95). HRMS, M⁺+Na, Found: 507.3665, Calcd for C₂₈H₅₂O₆Na, 507.3662.

3-O-dimethylphosphoryl-1-O-Oleoyl-2-O-(tetrahydro-pyran-2-yloxy)ethyl-sn-glycerol (1-7a). To a solution of 6 (55 mg, 0.113 mmol) in CH₂Cl₂ (5 mL) in an ice bath was added (OMe)₂POCL (20 mg, 0.136 mmol), t-BuOK (19 mg, 0.17 mmol). The reaction mixture was stirred at rt for 2 h. NH₄Cl saturated solution (2 mL) was added and the mixture was stirred for 10 min. The reaction mixture was extracted with CH₂Cl₂, the extract was washed with NaCl saturated solution, dried over Na₂SO₄, and concentrated. FC (EtOAc/Hexane, 2/1, v/v) gave 1-7a as a colorless oil 50 mg (75%). R_{f}0.26 (EtOAc/Hexane 2/1); ¹H-NMR (CDCl₃, 400 MHz), δ 0.875 (t, J=6.8 Hz, 3H), 1.280 (m, 20H), 1.499˜1.819 (m, 8H), 2.004 (m, 2H), 2.32 (t, J=8 Hz, 2H), 3.529 (m, 2H), 3.71˜3.872 (m, 11H), 4.128 (m, 2H), 4.247 (m, 2H), 4.62 (t, J=4.4, 1H), 5.34 (m, 2H); MS (FAB) m/z 615 (M⁺+Na, 100). HRMS, M⁺+Na, Found: 615.3646, Calcd for C₃₀H₅₇O₉NaP, 615.3638.

2-O-hydroxyethyl-1-O-oleoyl-3-O-phosphoryl-sn-glycerol (1-8a). A solution of 1-7a (35 mg, 0.069 mmol), TMSBr (37 mg, 0.24 mmol) in CH₂Cl₂ (1 mL) was stirred at rt for 5 h. The solvent was evaporated and the residue was dissolved in 95% methanol (1 mL) stirring for 1 h. Reconcentration of the solvent gave 1-8a as a colorless oil 32 mg (95%). R_{f}0.36 (CH₂Cl₂/MeOH/H₂O, 20/10/1); ¹H-NMR (CD₃OD), δ 0.893 (t, J=7.2 Hz, 3H), 1.304 (m, 20H), 1.609 (m, 2H), 2.024 (m, 4H), 2.341 (t, J=7.6 Hz, 2H), 3.667 (m, 4H), 3.787 (m, 1H), 4.049 (m, 2H), 4.2 (m, 2H), 5.336 (m, 2H); ¹³C-NMR (CD₃OD), δ 14.452, 23.74, 25.990, 28.125, 30.192, 30.299, 30.337, 30.444, 30.611, 30.81, 30.840, 33.059, 34.912, 62.42, 63.914, 66.56 (d, J=5.35 Hz), 72.974, 77.985 (d, J=7.78 Hz), 130.795, 130.894, 175.163. ³¹P-NMR (CD₃OD), δ 1.078 (s).

2-O-hydroxyethyl-1-O-palmitoyl-3-O-phosphoryl-sn-glycerol (1-8b). R_{f}0.36 (CH₂Cl₂/MeOH/H₂O, 20/10/1); ¹H-NMR (CD₃OD), δ 0.891 (t, J=7.2 Hz, 3H), 1.281 (s, 24H), 1.608 (m, 2H), 2.34 (t, J=7.2 Hz, 2H), 3.670 (m, 4H), 3.799 (m, 1H), 4.054 (m, 2H), 4.2 (m, 2H); ¹³C-NMR, δ; ³¹P-NMR, δ 1.078 (s)

3-O-(tetrahydro-pyran-2-yloxy)ethyl-sn-glycerol (2-1). R_{f}0.25 (EtOAc); ¹H-NMR (CDCl₃) δ 1.521 (m, 4H), 1.78 (m, 2H), 2.710 (s, 1H), 3.332 (s, 1H), 3.51 (m, 2), 3.56˜3.70 (m, 6H), 3.857 (m, 3H), 4.610 (t, J=4 Hz, 1H); ¹³C-NMR, δ 19.508 (d, J=1.15 Hz), 25.299, 30.523, 62.503 (d, J=3.8 Hz), 63.975 (d, J=2.2 Hz), 66.732 (d, J=4.6 Hz), 70.423 (d, J=3.0 Hz), 70.846 (d, J=5.4 Hz), 73.016 (d, J=7.6 Hz), 99.166 (d, J=4.5 Hz).

1,2-di-O-tert-butyl-dimethysilyl-3-O-(tetrahydro-pyran-2-yloxy)ethyl-sn-glycerol (2-2). A mixture of 2-1 (400 mg, 1.8 mmol), tert-butyldimethylsilyl chloride (663 mg, 4.4 mmol) and imidazole (272 mg, 4 mmol) in anhydrous DMF (6 mL) under an argon atmosphere was stirred at rt for 20 h. The reaction mixture was diluted with H₂O (5 mL) and extracted with EtOAc (3×10 mL). The combined organic layers were dried over Na₂SO₄, and concentrated. FC (EtOAc/Hexane, 1/8, v/v) gave 2-2 as a colorless oil 730 mg (91%). R_{f}0.43 (EtOAc/Hexane 1/8); ¹H-NMR (CDCl₃) δ 0.068 (m, 12H), 0.883 (m, 18H), 1.483˜1.856 (m, 6H), 3.423 (m, 2H), 3.48˜3.65 (m, 6H), 3.839 (m, 3H), 4.632 (t, J=3.6 Hz, 1H); ¹³C-NMR, δ−5.436, −5.375, −4.681, −4.635, 18.190, 18.335, 19.319, 19.380, 25.458, 25.831, 25.862, 25.946, 30.545 (d, J=1.5 Hz), 62.010 (d, J=9.1 Hz), 65.167, 65.949 (d, J=4.6 Hz), 70.745 (d, J=5.4 Hz), 72.709, 73.334 (d, J=3.0 Hz), 98.866 (d, J=12.2 Hz).

2-O-tert-butyl-dimethysilyl-3-O-(tetrahydro-pyran-2-yloxy)ethyl-sn-glycerol (2-3). The HF-pyridine complex (0.383 mL, 13.2 mmol) was added to a mixture of 2-2 (1.0 g, 2.2 mmol) and pyridine (1.15 mL) in anhydrous THF (10 mL). After stirring 20 h at rt, the solution was diluted with EtOAc (50 mL), washed with 0.5M HCl (2×10 mL) and satd. CuSO₄ solution (10 mL). The organic layer was dried over Na₂SO₄, and concentrated. FC (EtOAc/Hexane, 1/2, v/v) gave 2-3 as a colorless oil 450 mg (58%). R_{f}0.35 (EtOAc/Hexane 1/2); ¹H-NMR (CDCl₃) 0.078 (s, 6H), 0.876 (s, 9H), 1.474˜1.848 (m, 6H), 2.321 (t, J=3.6 Hz, 1H), 3.455˜3.645 (m, 8H), 3.872 (m, 3H), 4.609 (t, J=3.2 Hz, 1H); ¹³C-NMR, δ−4.901, −4.665, 18.076, 19.319, 19.365, 25.367, 25.763, 30.468, 62.125 (d, J=6.1 Hz), 65.041 (d, J=3.8 Hz), 66.510 (d, J=6.1 Hz), 70.711 (d, J=4.6 Hz), 71.039 (d, J=3.0 Hz), 73.194 (d, J=8.3 Hz), 98.905 (d, J=10.7 Hz).

1-O-(tetrahydro-pyran-2-yloxy)ethyl-2-O-tert-butyl-dimethysilyl-3-O -dimethylphosphoryl-sn-glycerol (2-4). Colorless oil. R_{f}0.35 (EtOAc/Hexane 2/1); ¹H-NMR (CDCl₃) 0.073 (d, J=2.4 Hz, 6H), 0.866 (s, 9H), 1.478˜1.829 (m, 6H), 3.542 (m, 4H), 3.62 (m, 2H), 3.733 (s, 3H), 3.764 (s, 3H), 3.835 (m, 2H), 3.967 (m, 2H), 4.077 (m, 1H), 4.601 (t, J=4.0 Hz, 1H); ¹³C-NMR, δ−4.874, −4.820, 18.058, 19.347, 19.385, 25.379, 25.684, 30.496, 54.183, 54.244, 62.103 (d, J=5.3 Hz), 65.610 (d, J=2.3 Hz), 69.008 (d, J=6.1 Hz), 70.761 (dd, J=8.4, 2.3 Hz), 70.850 (d, J=2.3 Hz), 72.264 (d, J=4.6 Hz), 98.906 (d, J=6.9 Hz); ³¹P-NMR, δ 2.379 (s).

3-O-dimethylphosphoryl-(2R)-O-oleoyl-1-O-tetrahydro-pyran-2-yloxy)ethyl-sn-glycerol (2-6a). R_{f}0.50 (EtOAc); ¹H-1MR (CDCl₃) δ 0.871 (t, J=6.8 Hz, 3H), 1.275 (m, 20H), 1.494˜1.832 (m, 8H), 2.004 (m, 2H), 2.328 (t, J=7.2 Hz, 2H), 3.542 (m, 4H), 3.579 (m, 2H), 3.664 (m, 6H), 3.858 (m, 2H), 4.223 (m, 2H), 4.611 (t, J=4.0 Hz, 1H), 5.171 (m, 1H), 5.334 (m, 2H); ¹³C-NMR, δ 14.083, 19.406, 22.655, 24.836, 25.393, 27.147, 27.193, 29.053, 29.091, 29.168, 29.297, 29.496, 29.686, 29.740, 30.525, 31.875, 34.231, 54.326, 54.387, 62.158, 65.983 (d, J=5.3 Hz), 66.551, 68.808, 70.486, 70.562, 70.882, 98.912 (d, J=3.8 Hz), 129.695, 129.992; ³¹P-NMR, δ 2.258 (s)

1-O-hydroxyethyl-2-O-oleoyl-3-O-phosphoryl-sn-glycerol (2-7a). R_{f}0.35 (CH₂Cl₂/MeOH/H₂O, 20/10/1); ¹H-NMR (CD₃OD) δ 0.893 (t, J=6.8 Hz, 3H), 1.305 (m, 20H), 1.614 (t, J=6.8 Hz, 2H), 2.024 (m, 4H), 2.347 (t, J=5.6 Hz), 3.555 (m, 2H), 3.645 (t, J=4.4 Hz, 2H), 3.708 (m, 2H), 4.14 (m, 2H), 5.145 (m, 1H), 5.337 (t, J=4.8 Hz, 2H); ¹³C-NMR, δ 13.260, 22.548, 24.775, 26.993, 28.954, 29.000, 29.153, 29.252, 29.419, 29.633, 29.656, 31.867, 33.865, 60.968, 64.698, 68.762, 71.252 (d, J=8.4 Hz), 72.796, 72.850, 129.610, 129.694; ³¹P-NMR, δ 1.012 (s).

1-O-hydroxyethyl-2-O-palmitoyl-3-O-phosphoryl-sn-glycerol (2-7b). R_{f}0.35 (CH₂Cl₂/MeOH/H₂O, 20/10/1); ¹H-NMR (CD₃OD) δ 0.890 (t, J=6.8 Hz, 3H), 1.280 (s, 24H), 1.601 (m, 2H), 2.346 (t, J=7.6 Hz, 2H), 2.567 (m, 2H), 3.634 (m, 2H), 3.717 (m, 2H), 4.143 (m, 2H), 5.147 (m, 1H); ¹³C-NMR, δ 14.431, 23.727, 25.969, 26.023, 30.156, 30.362, 30.423, 30.469, 30.560, 30.598, 30.675, 30.751, 30.781, 62.155, 65.937, 70.048, 72.801, 73.853, 74.010 (d, J=5.3 Hz); ³¹P-NMR, δ 0.957 (s).

IV. Synthesis of α-Fluorinated Phosphonates

One approach toward the target α-monofluorophosphonates involved the Wadsworth-Emmons condensation of carbanion, derived from tetraalkyl monofluoromethylenediphosphonates, with (R)-1,4-dioxaspiro[4,5]decane-2-carbaldehyde. The cyclohexyl protecting group in the aldehyde increased the stereoselectivity of condensation because the preferred conformation of vinylphosphonate had the most bulky β-carbon substituent trans to the phosphoryl group. The use of Selectfluor(1-chloromethyl-4-fluoro-1,4-diazobicyclo[2.2.2]octane bis(tetrafluoroborate), F-TEDA-BF₄) (Lal, J. Org. Chem., 1993, 57, 4676-4683; Lal etal. Chem. Rev. 1996, 96, 1737-1755) was selected in the synthesis of tetraethyl fluoromethylenebisphosphonate. The tetraethyl methylenebisphosphonate was treated with sodium hydride, and the enolate was quenched with Selectfluor to give the tetraethyl fluoromethylenebisphosphonate 2 in good yield (52%).

Treatment of compound 2 with n-butyl lithium at −78° C. generated the lithiated carbonion, which condenses smoothly with aldehyde 3 giving good yield of the α-fluorovinylphosphonate (FIG. 8). The condensation reaction showed a good stereoselectivity and gave a mixture of (E)- and (Z)-isomers in a 12:1 (mol ratio). Moreover, these two isomers can be separated easily by flash chromatograph. Their stereochemistry Were confidently assigned on the basis of the ³J_{PH} and ³J_{HF} coupling constants for the alkene.

Catalytic hydrogenation of the alkene 4, proceeded readily and quantitatively to give the corresponding α-fluoroalkylphosphonate 5 without loss of fluorine (FIG. 8). The hydrogenation was carried out at ambient temperature and pressure using 10% Pd-C in absolute ethanol. Hydrolysis 5 using catalytic amount of p-toluenesulfonic acid in MeOH cleaved the acetonide protecting group readily. DCC-promoted esterification of diol 6 with palmatic acid, oleic acid or linoleic acid provided good yield of ester 7a, 7b and 7c, respectively. Finally, treatment 7 with bromotrimethylsilane and subsequent addition of aqueous methanol (5%, H₂O) provided the desired fluorinated lysophosphatidic acid 8 in nearly quantitative yield.

The study on the LPA receptors/ligand interactions indicated introduction of sn-2 O-methyl group decreasing the ability to activate Edg4/LPA₂ receptor and increasing the Edg7/LPA₃ receptor subtype selectivity. For example, OMPT, a phospothionate analogue of LPA, exhibits preferred selectivity for Edg7/LPA₃ as compared to Edg2/LPA₁ or Edg4/LPA₂. In addition, selective introduction of O-methyl group at the sn-1 position can generate stable (acyl migration blocked) 2-acyl LPA analogues, which are a kind of important LPA species (Xu et al. Clinical Cancer Research 1995, 1, 1223-1232). In order to increase the subtype selectivity of analogs 8, the introduction of an O-methyl group at the sn-2 and sn-1 position was performed.

Selective introduction of a TBS protecting group at the sn-1 position of 6 was achieved by using 1.05 equivalent of TBSCl to produce 9 (FIG. 9). Next, the use of Meerwein's trimethyloxonium tetrafluoroborate salts (CH₃)₃O⁺BF₄⁻ in conjunction with nonnucleophilic amine base (proton sponge, 1,8-bis(dimethylamino)naphthalene) gave a medium yield (43%) of methyl ether 10 after 14 days together with unreacted starting material. Alternatively, the reaction of trimethyloxonium tetrafluoroborate salts (CH₃)₃O⁺BF₄⁻ with diol 6 in the presence of proton sponge provided good yield of 1-O-methylation product 11 after 4 days reaction at room temperature (FIG. 9). After esterification at sn-2 position and deprotection of diethyl ester, the acyl-chain migration-blocked sn-2 LPA analogues 13 were obtained.

Another approach to compound 10 involves the use of trimethylsilyldiazomethane TMSCHN₂, which smoothly reacts with alcohol 9 in dichloromethane in the presence of 42% aqueous fluoroboric acid (FBA) to give the corresponding methyl ether 10 in good yield. The stable TBDMS ether 10 was deprotected with tetra-(n-butyl)ammonium fluoride (TBAF) in THF to give the primary alcohol 14 (FIG. 10); neutralization of TBAF with acetic acid inhibited the side-effect of basic medium. DCC-promoted esterification of 14 with either oleic acid or palmatic acid provided good yields of esters 15. Finally, treatment of each ester 15 with bromotrimethylsilane and subsequent addition of 5% aq. methanol provided the desired sn-2 O-methylation LPA analogues 16 in nearly quantitatively yield. Moreover, the excessive TMSBr did not cleave off O-methyl ether.

Trimethylsilyldiazomethane TMSCHN₂ reacted with alcohol 9 smoothly to give methyl ether 10. Using a similar approach, it was possible to go directly from alcohol 7 to compound 15. The reaction of trimethylsilyldiazomethane TMSCHN₂ with alcohol 7 provided good yield of 15 and no migration of acyl chain was observed (FIG. 10). This method not only saved several steps for the synthesis of sin-2 O-methylation LPA analogs, but also provided a new and concise synthetic route for the construction of this kind of compound.

General Procedure. Chemicals were obtained from Aldrich and Acros Chemical Corporation and were used without prior purification. Solvents used were of reagent grade and were distilled before use: THF was distillated from sodium wire. Methylene chloride was distillated from CaH₂. Reactions were performed under an inert atmosphere (N₂ or Ar) unless otherwise indicated. ¹H and ¹³C spectra were recorded on 400 MHz (¹H), 101 MHz (¹³C), 162 MHz (³¹P) and 376 MHz (¹⁹F), temp. 25° C. Chemical shifts are given in ppm with TMS as internal standard (δ=0.00); ³¹P, 85% H₃PO₄ (δ=0.00); ¹⁹F, CFCl₃ (δ=0.00). (R)-1,4-Dioxaspiro[4,5]decane-2-carbaldehyde was prepared from 1,2:5,6-Di-O-cyclohexylidene-D-mannitol according to Schick's method. (Schrotter, E.; Luong, T. T.; Schick, H. J. Prakt. Chemie. 1990, 332, 191-197).

Tetraethyl fluoromethylenebisphosphonate 2. NaH (0.641 g, 16.03 mmol, 60% in mineral oil) in a flame-dried flask under Ar was washed with Et₂O, and dried THF (90 mL) was added. The suspension was cooled (˜0° C., ice bath), and compound 2 (4.40 g, 15.26 mmol) in THF (10 mL) was added. The solution was stirred (0° C. for 15 min, ambient temperature for 60 min, cooled to 0° C.), and selectfluor (6.76 g, 19.08 mmol) was added in one portion. After 15 min, dried DMF (35 mL) was added, the ice-bath was removed after 5 min, and stirring was continued at ambient temperature for 2 h. The reaction mixture was cooled to 0° C., and CH₂Cl₂ (40 mL) and saturated NH₄Cl/H₂O (40 mnL) were slowly added. After 5 min, the organic layer was separated, and the aqueous layer was extracted (CH₂Cl₂). The combined organic phase was washed (saturated NaHCO₃/H₂O, brine), dried (MgSO₄), evaporated, and chromatographed(Ethyl acetate/CH₃OH:100/3, R_{f}=0.54, 2.40 g, 7.84 mmol, 52% yield). ¹H NMR(CDCl₃): 4.93 (dt, J=46.0, 13.6 Hz,1H), 4.20 (m, 8H), 1.29 (t, J=7.2 Hz, 12H). ¹⁹F NMR (CDCl₃): −288.26 (td, J=62.9, 45.9 Hz, 1F). ³¹P NMR (CDCl₃): 12.20 (d, J=63.0 Hz).

(E)-(3R)-Diethyl 1-Fluoro-3,4-O-cyclohexylidene-3,4-dihydroxybut-1-enylphosphonate 4a. Treatment of tetraethyl fluoromethylenebisphosphonate (0.184 mg, 0.601 mmol in 5 mL dry hexane) with n-BuLi (0.601 mL, 1.0 M solution in hexane) at −78° C. under dry nitrogen gas followed by addition of (R)-1,4-dioxaspiro[4,5]decane-2-carbaldehyde (0.143 g, 0.841 mmol) with stirring at −78° C. gave a mixture which was brought to room temperature slowly. Filtration and evaporation under reduced temperature, followed by chromatograph (Ethyl acetate/hexane: 3/2) gave two isomers 4a (R_{f}=0.19, 0.178 g, 0.553 mmol, 92%) and 4b (R_{f}=0.25, 0.015 g, 0.047 mmol, 7%). ¹H NMR (CDCl₃): 5.99 (dt, J=39.2, 7.6 Hz, 1H), 4.98 (m, 1H), 4.17-4.08 (m, 5H), 3.63 (dd, J=7.6, 6.4 Hz, 1H), 1.56 (m, 10H), 1.32 (m, 6H). ¹³C NMR (CDCl₃): 151.85 (dd, J=278.0, 233.2 Hz), 124.36 (dd, J=27.6, 3.0 Hz), 110.6 (s), 68.67 (dd, J=12.3, 6.9 Hz), 68.45 (m), 63.29 (dd, J=5.3, 3.0 Hz), 36.09 (s), 35.17 (s), 24.97 (s), 23.78 (s), 16.17 (d, J=6.1 Hz). ¹⁹F NMR (CDCl₃): −127.04 (dd, J=99.0, 39.1 Hz, 1F). ³¹P NMR (CDCl₃): 4.68 (d, J=98.9 Hz). MS (CI) m/z 323 (M⁺+1, 69.89), 99 (OC₆H₁₁⁺, 100.00). HRMS, M⁺, Found: 322.1354. Calcd for C₁₄H₂₄FO₅P, 322.1345. [α]²⁰_{D}=+51.68 (c=0.15, EtOH).

(Z)-(3R)-Diethyl 1-Fluoro-3,4-O-cyclohexylidene-3,4-dihydroxybut-1-enylphosphonate 4b. ¹H NMR (CDCl₃): 6.08 (ddd, J=30.8, 26.8, 9.6 Hz, 1H), 5.41 (m, 1H), 4.16 (m, 5H), 3.62 (dd, J=8.0, 6.0 Hz, 1H), 1.59 (m, 8H), 1.34 (m, 8H) ¹⁹F NMR (CDCl₃): −118.34 (dd, J=101.6, 26.3 Hz, 1F). ³¹P NMR (CDCl₃): 3.74 (d, J=101.0 Hz).

(3R)-Diethyl 1-Fluoro-3,4-O-cyclohexylidene-3,4-dihydroxybut-1-phosphonate 5. A solution of 4 (0.128 g, 0.398 mmol) in absolute ethanol (8 mL) containing 10% Pd-C catalyst (10 mg) was stirred at ambient temperature under hydrogen (1 atm) until gas uptake ceased (18 h). Filtration and evaporation under reduced pressure gave compound 5 as a colourless liquid (0.126 g, 0.390 mmol, 98% yield). ¹H NMR (CDCl₃): 4.99-4.76 (m, 1H), 4.33-4.01 (m, 5H), 3.63-3.54 (m, 1H), 2.25-1.98 (m, 2H), 1.56 (m, 8H), 1.31 (m, 8H). ¹³C NMR (CDCl₃): 109.70 (s), 109.66 (s), 86.14 (dd, J=179.4, 171.8 Hz), 86.00 (dd, J=179.4, 171.8 Hz), 71.92 (dd, J=11.5, 3.0 Hz), 71.27 (dd, J=11.5, 3.0 Hz), 68.94 (s), 68.33 (s), 63.09 (dd, J=39.9, 6.9 Hz), 62.98 (dd, J=33.7, 4.6 Hz), 36.70 (s), 36.1417 (s), 35.06 (s), 34.81 (s), 33.99 (d, J=19.1 Hz), 16.40 (d, J=6.1 Hz). ¹⁹F NMR (CDCl₃): −207.52 (m), −212.53 (m). ³¹P NMR (CDCl₃): 18.76 (d, J=73.8 Hz), 18.47 (d, J=73.8 Hz). MS (CI) m/z 325 (M⁺+1, 100.00). HRMS, M⁺, Found: 324.1519. Calcd for C₁₄H₂₆FO₅P, 324.1502. [α]²⁰_{D=-}5.59 (c=0.34, EtOH).

(3R)-Diethyl 1-Fluoro-3,4-dihydroxybut-1-phosphonate 6. TosOH (7 mg, 0.035 mmol, 0.10 eq.) was added to a solution of 5 (0.114 g, 0.352 mmol) in MeOH (5 mL), and the solution was stirred at room temperature for 24 h. After addition of solid NaHCO₃ to neutralize the reaction mixture, the solvent was removed under reduced pressure. Chromatograph got pure product (75 mg, 0.306 mmol, 87%). ¹H NMR (CDCl₃): 5.11-4.87 (m, 1H), 4.19-4.08 (m, 5H), 3.96 (br, 1H), 3.79 (br, 1H), 3.59 (m, 1H), 3.40 (m, 1H), 2.15-1.77 (m, 2H), 1.30 (t, J=6.8 Hz, 8H). ¹⁹F NMR (CDCl₃): −207.43 (m), −211.70 (m). ³¹P NMR (CDCl₃): 19.89 (d, J=74.0 Hz), 19.36 (d, J=75.9 Hz). [α]²⁰_{D}32 −13.42 (c=0.73, EtOH).

Diethyl [1-fluoro-3 (S)-hydroxyl-4-(oleoyloxy)butyl]Phosphonate 7a. To the alcohol solution 6 and (42 mg, 47 μL, 0.147 mmol) of oleic acid in dry CH₂Cl₂ (1 mL) at rt was added dropwise a solution of DCC (30 mg, 0.147 mmol) and DMAP (6 mg, 0.048 mmol) in dry CH₂Cl₂ (1 mL). The solution was stirred at rt for 18 h and filtered, the solvent removed, and the residue was purified by chromatography (n-hexane/ethyl acetate 1:1, R_{f}=0.28) to afford a waxy solid 12 mg. (0.026 mmol, 45%). ¹H NMR (CDCl₃): 5.29 (m, 2H), 5.10-4.89 (m, 1H), 4.22-3.98 (m, 7H), 3.48 (br, 1H), 2.29 (t, J=7.6 Hz, 2H), 2.18-2.03 (m, 2H), 1.93 (m, 4H), 1.58 (m, 2H), 1.33-1.22 (m, 28H), 0.83 (t, J=7.2 Hz, 3H). ¹³C NMR(CDCl₃): 173.84 (s), 173.81 (s), 129.92 (s), 129.64 (s), 86.49 (dd, J=171.0, 172.6 Hz), 84.71 (dd, J=171.1, 172.6 Hz), 68.06 (s), 67.48 (s), 66.01 (dd, J=10.0, 3.8 Hz), 65.07 (dd, J=13.1, 3.0 Hz), 63.55 (d, J=6.9 Hz), 63.30 (d, J=6.9 Hz), 63.06 (d, J=6.9 Hz), 62.98 (d, J=8.4 Hz), 34.36 (d, J=19.9 Hz), 33.81 (d, J=18.4 Hz), 31.82 (s), 29.67 (s), 29.61 (s), 29.43 (s), 29.23 (s), 29.09 (s), 27.13 (s), 27.08 (s), 24.86 (s), 22.59 (s), 16.35 (m), 14.02 (s). ¹⁹F NMR (CDCl₃): −208.26 (1F, m), −211.75 (1F, m). ¹³P NMR (CDCl₃): 19.36 (d, J=73.8 Hz), 19.10 (d, J=76.1 Hz). MS (CI) m/z 509.4 (M⁺+1, 29.75), 463.3 (M⁺-OC₂H₅, 100.00). HRMS, M⁺+1, Found: 509.3400. Calcd for C₂₆H₅₁FO₆P, 509.3407. [α]²⁰_{D}=−2.61 (c=2.38, MeOH).

Diethyl [1-fluoro-3 (S)-hydroxyl4-(linoleoyloxy)butyl]Phosphonate 7b. Yield 61%. ¹H NMR (CDCl₃): 5.30 (m, 4H), 5.10-4.90 (m, 1H), 4.17-4.01 (m, 7H), 3.51 (br, 0.5H), 3.24 (br, 0.5H), 2.70 (m, 2H), 2.29 (t, J=6.8 Hz, 3H), 2.15-1.98 (m, 6H), 1.57 (m, 2H), 1.29 (m, 20H), 0.83 (t, J=6.4 Hz, 3H). ¹³C NMR (CDCl₃): 173.77 (s), 130.10 (s), 129.91 (s), 127.95 (s), 127.80 (s), 85.95 (dd,J=178.7, 171.1 Hz), 85.19 (dd, J=179.5, 171.3 Hz), 68.02 (s), 67.45 (s), 65.99 (dd, J=9.3, 3.9 Hz), 65.00 (dd, J=9.8, 9.7 Hz), 63.40 (dd, J=25.5, 6.8 Hz), 63.00 (dd, J=6.8, 6.8 Hz), 34.14 (dd, J=41.4, 19.2 Hz), 31.41 (s), 29.49 (s), 29.24 (s), 29.07(s), 29.00 (s), 27.09 (s), 25.52 (s), 24.78 (s), 22.46 (s), 16.36 (d, J=4.5 Hz), 13.96 (s). ¹⁹F NMR (CDCl₃): −208.25 (m), −211.79 (m). ³¹P NMR (CDCl₃): 19.37 (d, J=73.8 Hz), 19.09 (d, J=76.1 Hz). MS (CI) m/z 507 (M⁺+1, 100.00), 463.3 (M⁺-OC₂H₅, 48.19). HRMS, M⁺, Found: 506.3174. Calcd for C₂₆H₄₈FO₆P, 506.3173. [α]²⁰_{D}=−4.29 (c=0.14, EtOH).

Diethyl [1-fluoro-3 (S)-hydroxyl-4-(palmitoyloxy)butyl]Phosphonate 7c. 51% yield. ¹H NMR (CDCl₃): 5.11-4.90 (m, 1H), 4.23-3.99 (m, 7H), 3.42 (br, 1H), 2.31 (t, J=7.6 Hz, 2H), 2.19-1.90 (m, 2H), 1.68-1.55 (m, 2H), 1.33 (t, J=6.8 Hz, 6H), 1.60 (m, 24H), 0.84 (t, J=7.2 Hz, 3H). ¹³C NMR (CDCl₃): 173.92 (s), 173.89 (s), 86.56 (dd, J=171.0, 168.2 Hz), 84.78 (dd, J=171.0, 168.2 Hz), 68.10 (s), 67.53 (s), 66.11 (dd, J=9.3, 3.8 Hz), 65.21 (dd, J=13.0, 3.1 Hz), 63.48 (dd, J=24.6, 6.9 Hz), 63.05 (dd, J=9.3, 6.8 Hz), 49.03 (s), 34.36 (d, J=19.9 Hz), 31.87 (s), 29.63 (s), 29.60 (s), 29.41 (s), 29.22 (s), 29.09 (s), 25.59 (s), 24.86 (s), 22.63 (s), 16.41 (d, J=5.3 Hz), 16.37 (d, J=4.6 Hz), 14.06 (s). ¹⁹F NMR (CDCl₃): −208.37 (1F, m), −211.62 (1F, m). ³¹P NMR (CDCl₃): 19.34 (d, J=73.8 Hz), 19.11 (d, J=76.1 Hz). MS (CI) m/z 483.4 (M⁺+1, 55.29), 437.4 (M⁺-OC₂H₅, 100.00). HRMS, M⁺+1, Found: 483.3244. Calcd for C₂₄₄₉FO₆P, 483.3251. [α]²⁰_{D}=−2.20 (c=1.00, MeOH).

[1-Fluoro-3 (S)-hydroxyl-4-(oleoyloxy)butyl]phosphonate 8a. Thoroughly dried (64 mg, 0.126 mmol, 5 h under high vacuum) was dissolved in anhydrous methylene chloride (1 mL) at room temperature. Bromotrimethylsilane (193 mg, 1.260 mmol) was added with a dry syringe and stirred 4 h. TLC indicated that all of the reactant had disappeared, then the solvent removed under reduced pressure and dried under vacuum. The residue was dissolved in 95% mehtanol (1 mL) for 1h, then the solvent removed under reduced pressure and dried under vacuum, got final product 55 mg. (0.121 mmol, 96% yield.). ¹H NMR (CD₃OD): 5.34 (m, 2H), 5.21-5.17 (m, 1H), 4.79 (m, 1H), 3.68 (dd, J=11.60, 4.40 Hz, 1H), 3.57 (m, 1H), 2.35 (m, 4H), 2.01 (m, 4H), 1.63 (m, 2H), 1.33-1.22 (m, 20H), 0.89 (t, J=7.2 Hz, 3H). ¹³C NMR (CD₃OD): 174.33 (s), 174.17 (s), 130.84 (s), 130.74 (s), 88.16 (dd, J=170.3, 168.7 Hz), 86.39 (dd, J=170.3, 168.7 Hz), 71.30 (dd, J=14.6, 2.3 Hz), 69.52 (dd, J=14.6, 2.3 Hz), 35.12 (d, J=19.3 Hz), 34.93 (d, J=18.9 Hz), 33.04 (s), 30.84 (s), 30.77 (s), 30.61 (s), 30.44 (s), 30.35 (s), 30.26 (s), 30.16 (s), 30.13 (s), 28.14 (s), 28.13 (s), 23.72 (s), 14.55 (s). ¹⁹F NMR (CD₃OD): −208.60 (1F, m), −210.99 (1F, m). ³¹P NMR (CD₃OD): 16.21 (d, J=72.7 Hz), 15.95 (d, J=73.8 Hz). MS (CI) m/z 435.3 (M⁺-OH, 60.85), 283.3 (M⁺-C₄H₉—CFH₃PO₃, 100.00). HRMS, M⁺-OH, Found: 435.2678. Calcd for C₂₂H₄₁FO₅P, 435.2676. [α]²⁰_{D}=−2.13 (c=0.14, MeOH).

[1-Fluoro-3 (S)-hydroxyl-4-(linoleoyloxy)butyl]phosphonate 8b. 93% yield. ¹H NMR (CD₃OD): 5.30 (m, 4H), 5.10-4.90 (m, 1H), 4.17-4.01 (m, 3H), 3.51 (br, 0.5H), 3.24 (br, 0.5H), 2.70 (m, 2H), 2.29 (t, J=6.8 Hz, 3H), 2.15-1.98 (m, 6H), 1.57 (m, 2H), 1.29 (m, 14H), 0.83 (t, J=6.4 Hz, 3H). ¹³C NMR??? (CD₃OD): 174.33 (s), 174.17 (s), 130.84 (s), 130.74 (s), 88.16 (dd, J=170.3, 168.7 Hz), 86.39 (dd, J=170.3, 168.7 Hz), 71.30 (dd, J=14.6, 2.3 Hz), 69.52 (dd, J=14.6, 2.3 Hz), 35.12 (d, J=19.3 Hz), 34.93 (d, J=18.9 Hz), 33.04 (s), 30.84 (s), 30.77 (s), 30.61 (s), 30.44 (s), 30.35 (s), 30.26 (s), 30.16 (s), 30.13 (s), 28.14 (s), 28.13 (s), 23.72 (s), 14.55 (s). ¹⁹F NMR (CD₃OD): −208.25 (m), −211.79 (m). ³¹P NMR (CD₃OD): 19.37 (d, J=73.8 Hz), 19.09 (d, J=76.1 Hz). HRMS, M⁺-OH, Found: 433.2502. Calcd for C₂₂H₃₉FO₅P, 433.2519. [α]²⁰_{D}=−2.78 (c=0.22, MeOH).

[1-Fluoro-3 (S)-hydroxyl-4-(palmitoyloxy)butyl]Phosphonate 8c. 91% yield. ¹H NMR(CD₃OD): 5.27-5.18 (m, 1H), 4.78 (m, 1H), 3,68 (dd, J=10.80, 4.00 Hz, 1H), 3.57 (m, 1H), 2.40-2.25 (m, 4H), 1.64 (m, 2H), 1.33-1.22 (m, 24H), 0.89 (t, J=7.2 Hz, 3H). ¹³C NMR(CDCl₃): 172.33 (s), 172.30 (s), 87.06 (dd, J=170.3, 168.7 Hz), 85.29 (dd, J=170.3, 168.7 Hz), 69.33 (dd, J=14.2, 2.4 Hz), 67.56 (dd, J=14.2, 2.4 Hz), 33.04 (d, J=7.7 Hz), 31.92 (s), 31.06 (s), 28.77 (s), 28.75 (s), 28.71 (s), 28.58 (s), 28.47 (s), 28.39 (s), 28.15 (s), 24.05 (s), 23.97 (s), 23.92 (s), 21.72 (s), 12.48 (s). ¹⁹F NMR(CDCl₃): −208.73 (1F, m), −211.07 (1F, m). ³¹P NMR(CDCl₃): 16.21 (d, J=72.7 Hz), 15.95 (d, J=73.8 Hz). MS (CI) m/z 409.2 (M⁺+1-OH—CH₃, 2.29), 225.2 (M⁺-C₁₄H₂₉—OH, 100.00). HRMS, M⁺-OH—CH₃, Found: 408.2432. Calcd for C₂₀H₃₈FO₅P, 408.2441. [α]²⁰_{D}=−1.83 (c=0.17, MeOH).

Diethyl [1-fluoro-3 (S)-hydroxyl-4-(tetra-butyldimethylsilyl)-butyl]Phosphonate 9. To a solution of phosphate 6 (0.386 g, 1.582 mmol) and tert-butyldimethylsilyl chloride (TBSCl) (0.250 g, 1.661 mmol, 1.05 eq.) in anhydrous CH₂Cl₂ (8 mL) was added 4-dimethylaminopyridine(DMAP) (0.010 g, 0.080 mmol, 0.05 eq.) and triethylamine (0.168 g, 1.661 mmol, 1.05 eq.). The reaction mixture was stirred at room temperature for 16 h. The solution was diluted with CH₂Cl₂ (20 mL), and the solution was washed with saturated NH₄Cl aqueous solution and brine. After drying with anhydrous Na₂SO₄, the organic layer was concentrated in vacuo. The residue was purified by chromatography (Ethyl acetate/hexane=1:1, R_{f}=0.13) to afford a colorless liquid (0.413 g, 1.155 mmol, 73%). ¹H NMR (CDCl₃): 5.12-4.88 (m, 1H), 4.19 (m, 4H), 3.96-3.82 (m, 1H), 3.67-3.43 (m, 2H), 2.83 (d, J=4.4 Hz, 0.5H), 2.60 (d, J=5.2 Hz, 0.5H), 2.23-1.79 (m, 2H), 1.33 (t, J=6.8 Hz, 6H), 0.89 (s, 9H), 0.04 (s, 6H). ¹³C NMR (CDCl₃): 86.43 (dd, J=178.7, 171.0 Hz), 85.63 (dd, J=178.7, 171.0 Hz), 68.47 (dd, J=10.0, 3.8 Hz), 67.10 (dd, J=13.0, 3.8 Hz), 66.96 (s), 66.39 (s), 63.26 (dd, J=15.3, 6.8 Hz), 62.86 (dd, J=9.3, 6.9 Hz), 33.81 (d, J=18.4 Hz), 25.81 (s), 18.24 (s), 18.22 (s), 23.78 (s), 16.49 (d, J=3.8 Hz), 16.38 (d, J=3.8 Hz), −5.43 (s), −5.47 (s). ¹⁹F NMR (CDCl₃): −207.18 (m), −211.77 (m). ³¹P NMR (CDCl₃): 19.60 (d, J=75.0 Hz), 19.24 (d, J=77.1 Hz). MS (CI) m/z 359.0 (M⁺+1, 100.00). HRMS, M⁺+1, Found: 359.1819. Calcd for C₁₄H₃₃FO₅PSi, 359.1819. [α]²⁰_{D}=−20.91 (c=0.88, EtOH).

Diethyl [1-fluoro-3 (S)-O-methyl-4-(tetra-butyldimethylsilyl)-butyl]Phosphonate 10.

Method A: To a vigorously stirred mixture of 9 (0.046 g, 0.136 mmol) and FBA (42% aqueous fluoroboric acid, 0.028 g, 20 μL) in CH₂Cl₂ (1 mL) was added TMSCHN₂ (2.0M hexane solution, 136 μL) at 0° C. The stirring was continued at 0° C., and three further portions of TMSCHN₂ (68 μL×3) were added dropwise at intervals of 20 min. The mixture was stirred at 0° C. for further 30 min and at rt for another 30 min, added 10% NaHCO₃ solution (0.1 mL). The organic layer was dried over Na₂SO₄ and concentrated. The residue was purified by chromatography (Ethyl acetate/hexane=2:3, R_{f}=0.31) to afford a colorless liquid (0.034 g, 0.091 mmol, 67%).

Method B: To a stirred mixture of 9 (0.022 g, 0.061 mmol) and proton sponge (1,8-bis(dimethylamino)naphthalene) (0.016 g, 0.073 mmol) in CH₂Cl₂ (1 mL) was added Meerwein's trimethyloxonium tetrafluoroborate (0.009 g, 0.061 mmol) at room temperature. The resulting solution was stirred at room temperature for 14 days before it was diluted with CH₂Cl₂ (2 mL) and quenched with water (0.1 mL). The solution was dried over Na₂SO₄ and concentrated. The residue was purified by chromatography (Ethyl acetate/hexane=2:3, R_{f}=0.31) to afford a colorless liquid (0.010 g, 0.027 mmol, 43%).

¹H NMR (CDCl₃): 5.04-4.89 (m, 1H), 4.19 (m, 4H), 3.70-3.58 (m, 2H), 3.46 (m, 1H), 3.42 (s, 1.5H), 3.37 (s, 1.5H), 2.14-1.79 (m, 2H), 1.31 (m, 6H), 0.89 (s, 9H), 0.04 (s, 6H). ¹³C NMR (CDCl₃): 86.43 (dd, J=178.7, 171.0 Hz), 85.63 (dd, J=178.7, 171.0 Hz), 64.68 (s), 64.40 (s), 63.08 (m), 62.75 (m), 58.46 (s), 57.59 (s), 32.67 (d, J=22.2 Hz), 31.77 (d, J=19.2 Hz), 25.84 (s), 18.25 (s), 18.22 (s), 16.42 (d, J=6.1 Hz), −5.46 (s). ¹⁹F NMR (CDCl₃): −207.71 (m), −212.49 (m). ³¹P NMR (CDCl₃): 19.76 (d, J=76.1 Hz), 19.23 (d, J=76.1 Hz). MS (CI) m/z 373.19 (M⁺+1, 100.00). HRMS, M⁺+1, Found: 373.1974. Calcd for C₁₅H₃₄FO₅PSi, 373.1975. [α]²⁰_{D}=−13.96 (c=0.48, EtOH).

Diethyl [1-fluoro-3 (S)-hydroxyl-4-methyl-butyl]Phosphonate 11. To a stirred mixture of 9 (0.022 g, 0.061 mmol) and proton sponge (1,8-bis(dimethylamino)naphthalene) (0.016 g, 0.073 mmol) in CH₂Cl₂ (1 mL) was added Meerwein's trimethyloxonium tetrafluoroborate (0.009 g, 0.061 mmol) at room temperature. The resulting solution was stirred at room temperature for 4 days before it was diluted with CH₂Cl₂ (2 mL) and quenched with water (0.1 mL). After evaporated the solution, ethyl acetate was added and the solution was washed with saturated NH4Cl. The solution was dried with anhydrous and concentrated. The residue was purified by chromatography (CH2Cl2/CH3OH=2:3, R_{f}=0.31) to afford a colorless liquid (0.010 g, 0.027 mmol, 43%). ¹H NMR (CDCl₃): 5.10-4.89 (m, 1H), 4.13 (m, 4H), 4.10-3.90 (m, 1H), 3.41-3.40 (m, 3H), 3.33 (s, 3H), 2.15-2.01 (m, 2H), 1.30 (m, 6H). ¹⁹F NMR (CDCl₃): −207.59 (m), −212.02 (m). ³¹P NMR (CDCl₃): 19.76 (d, J=76.1 Hz), 19.23 (d, J=76.1 Hz).

Diethyl [1-fluoro-3 (S)-(oleoyloxy)-4-O-methyl-butyl]Phosphonate 12a. To a solution of alcohol 11 (0.036 g, 0.140 mmol) and oleic acid (0.043 g, 0.154 mmol) in dry CH₂Cl₂ (2 mL) was added a solution of DCC (0.040 g, 0.196 mmol) and DMAP (0.010 g, 0.084 mmol) in dry CH₂Cl₂ (4 mL) at 0° C. The solution was stirred for 16 h at rt, filtered, concentrated in vacuo, and the residue was purified on silica gel (n-hexane/ethyl acetate, HE: AE=1:1, R_{f}=0.34) to afford ester. (0.061 g, 0.117 mmol, 83%) as a waxy solid. ¹H NMR (CDCl₃): 5.31 (m, 2H), 5.21-5.16 (m, 1H), 4.93-4.77 (m, 1H), 4.19 (m, 4H), 3.49 (m, 1H), 3.43 (m, 1H), 3.32 (s, 3H), 2.32-2.13 (m, 4H), 1.98 (m, 4H), 1.59 (m, 2H), 1.34-1.23 (m, 26H), 0.84 (t, J=6.8 Hz, 3H). ¹³C NMR (CDCl₃): 173.20 (s), 173.07 (s), 129.95 (s), 129.69 (s), 84.85 (dd, J=178.7, 171.0 Hz), 84.05 (dd, J=178.7, 171.0 Hz), 73.46 (s), 73.03 (s), 69.35 (d, J=14.6 Hz), 67.95 (d, J=15.4 Hz), 63.32 (d, J=6.8 Hz), 62.97 (d, J=6.2 Hz), 59.16 (d, J=4.6 Hz), 34.33 (s), 34.28 (s), 31.85 (s), 31.76 (s), 29.71 (s), 29.65 (s), 29.47 (s), 29.27 (s), 29.13 (s), 29.07 (s), 29.02 (s), 27.16 (s), 27.13 (s), 24.92 (s), 24.83 (s), 16.41 (m), 14.05 (s). ¹⁹F NMR (CDCl₃): −208.71 (m), −211.47 (m). ³¹P NMR (CDCl₃): 18.57 (d, J=73.8 Hz), 18.21 (d, J=76.1 Hz). MS (CI) m/z 523.4 (M⁺+1, 100.00). HRMS, M⁺+1, Found: 523.3586. Calcd for C₂₇H₅₃FO₆P, 523.3564.

Diethyl [1-fluoro-3 (S)-(palmitoyloxy)-4-O-methyl-butyl]Phosphonate 12b. Same procedure as 12a, 87%. ¹H NMR (CDCl₃): 5.21 (m, 1H), 4.99-4.65 (m, 1H), 4.15 (m, 4H), 3.54 (m, 1H), 3.42 (m, 1H), 3.28 (s, 3H), 2.31-2.09 (m, 4H), 1.57 (m, 2H), 1.31 (m, 4H), 1.17 (m, 26H), 0.84 (t, J=6.8 Hz, 3H). ¹³C NMR (CDCl₃): 173.14 (s), 173.05 (s), 84.81 (dd, J=178.7, 171.0 Hz), 84.00 (dd, J=178.7, 171.0 Hz), 73.41 (s), 72.98 (s), 69.31 (d, J=14.6 Hz), 67.90 (d, J=15.4 Hz), 63.27 (d, J=6.8 Hz), 62.91 (d, J=6.2 Hz), 59.11 (d, J=4.6 Hz), 34.13 (s), 34.12 (s), 32.95 (s), 29.63 (s), 29.60 (s), 29.41 (s), 29.30 (s), 29.21 (s), 29.08 (s), 24.87 (s), 22.61 (s), 16.40 (d, J=5.3 Hz), 14.06 (s). ¹⁹F NMR (CDCl₃): −208.65 (m), −211.49 (m). ³¹P NMR (CDCl₃): 18.51 (d, J=73.7 Hz), 18.15 (d, J=75.4 Hz). MS (CI) m/z 497.4 (M⁺+1, 100.00). HRMS, M⁺+1, Found: 497.3398. Calcd for C₂₅H₅₁FO₆P, 497.3407.

[1-Fluoro-3(S)-(oleoyloxy)-4-O-methyl-butyl]Phosphonate 13a. 93% yield. ¹H NMR (CD₃OD): 5.34 (m, 2H), 5.26-5.22 (m, 1H), 4.91-4.4.40 (m, 1H), 3.57 (m, 1H), 3.47 (m, 1H), 3.36 (s, 3H), 2.37-2.13 (m, 4H), 2.02 (m, 4H), 1.61 (m, 2H), 1.32-1.29 (m, 22H), 0.89 (t, J=6.4 Hz, 3H). ¹³C NMR (CD₃OD): 172.89 (s), 172.72 (s), 128.90 (s), 128.87 (s), 86.33 (dd, J=178.7, 171.0 Hz), 85.52 (dd, J=178.7, 171.0 Hz), 72.76 (s), 72.24 (s), 69.25 (s), 69.11 (s), 57.36 (s), 33.20 (s), 33.13 (s), 31.06 (s), 30.95 (s), 28.84 (s), 28.80 (s), 28.61 (s), 28.45 (s), 28.35 (s), 28.29 (s), 28.18 (s), 28.11 (s), 26.13 (s), 24.09 (s), 21.74 (s), 12.46 (s). ¹⁹F NMR (CD₃OD): −208.66 (m), −211.40 (m). ³¹P NMR (CD₃OD): 16.64 (s), 16.22 (s). MS (CI) m/z 449.2 (M⁺+1-H₂O, 100.00). HRMS, M⁺+1, Found: 449.2824. Calcd for C₂₃H₄₃FO₅P, 449.2832.

[1-Fluoro-3(S)-(palmitoyloxy)-4-O-methyl-butyl]Phosphonate 13b. 95% yield. ¹H NMR (CD₃OD): 5.22 (m, 1H), 4.98-4.66 (m, 1H), 3.61 (m, 1H), 3.48 (m, 1H), 3.37 (s, 3H), 2.34 (t, J=6.0 Hz, 2H), 2.13-1.99 (m, 2H), 1.61 (m, 2H), 1.34 (m, 26H), 0.89 (t, J=6.8 Hz, 3H). ¹³C NMR (CD₃OD): 175.15 (s), 86.40 (dd, J=178.7, 171.0 Hz), 85.59 (dd, J=178.7, 171.0 Hz), 77.14 (s), 75.72 (s), 65.83 (s), 65.64 (s), 58.34 (s), 57.70 (s), 33.02 (d, J=7.7 Hz), 31.90 (s), 31.03 (s), 28.76 (s), 28.78 (s), 28.73 (s), 28.56 (s), 28.45 (s), 28.36 (s), 28.14 (s), 24.02 (s), 23.96 (s), 23.90 (s), 21.70 (s), 12.47 (s). ¹⁹F NMR (CD₃OD): −207.41 (m), −212.34 (m). ³¹P NMR (CD₃OD): 17.34 (d, J=73.7 Hz), 17.26 (d, J=76.1 Hz). MS (CI) m/z 423.2 (M⁺-OH, 79.26), 185.0 (M⁺C₁₅H₃₁CO₂H, 100.00). HRMS, M⁺+1, Found: 423.2671. Calcd for C₂₁H₄₁FO₅P, 423.2676.

Diethyl [1-fluoro-3 (S)-O-methyl-4-hydroxyl-butyl]Phosphonate 14. A solution of 10 (0.024 g, 0.063 mmol) in THF (1 mL) was treated successively with acetic acid (15 μL, 0.254 mmol) and tetrabutylammoniumfluoride trihydrate (0.080 g, 0.254 mmol) at room temperature. After stirring for 16 h, the reaction was completed (TLC control), then the solvent was evaporated under reduced pressure and the crude product was purified by pass through a short column (CH₂Cl₂/CH₃OH=30:1, R_{f}=0.13) to afford a colorless liquid (0.015 g, 0.059 mmol, 93%). ¹H NMR (CDCl₃): 5.02-4.79 (m, 1H), 4.18 (m, 4H), 3.83-3.67 (m, 1H), 3.59-3.46 (m, 2H), 3.42 (s, 1.5H), 3.38 (s, 1.5H), 2.21-1.98 (m, 3H), 1.35 (m, 6H). ¹³C NMR (CDCl₃): 85.66 (dd, J=184.8, 177.9 Hz), 63.32 (s), 63.15 (s), 62.92 (m), 57.90 (s), 57.14 (s), 32.29 (d, J=19.9 Hz), 30.64 (d, J=18.4 Hz), 16.43 (m). ¹⁹F NMR (CDCl₃): −207.03 (m), −211.39 (m). ³¹P NMR (CDCl₃): 19.40 (d, J=75.0 Hz), 18.89 (d, J=75.0 Hz).

Diethyl [1-fluoro-3 (S)-O-methyl-4-(oleoyloxy)-butyl]Phosphonate 15a.

Method A: To a vigorously stirred mixture of 7a (0.030 mg, 0.059 mmol) and FBA (42% aqueous fluoroboric acid, 0.012 g, 9 μL) in CH₂Cl₂ (1 mL) was added TMSCHN₂ (2.0M hexane solution, 59 μL) at 0° C. The stirring was continued at 0° C., and three further portions of TMSCHN₂ (30 μL×3) were added dropwise at intervals of 20 min. The mixture was stirred at 0° C. for further 30 min and at rt for another 30 min, added 10% NaHCO₃ solution (0.1 mL). The organic layer was dried over Na₂SO₄ and concentrated. The residue was purified by chromatography (Ethyl acetate/hexane=1:2, R_{f}=0.11) to afford a colorless liquid (0.026 g, 0.051 mmol, 86%).

Method B: To a solution of diol (0.016 g, 0.063 mmol) and oleic acid (0.020 g, 0.069 mmol) in dry CH₂Cl₂ (1 mL) was added a solution of DCC (0.016 g, 0.076 mmol) and DMAP (0.005 g, 0.038 mmol) in dry CH₂Cl₂ (1 mL) at 0° C. The solution was stirred for 16 h at rt, filtered, concentrated in vacuo, and the residue was purified on silica gel (n-hexane/ethyl acetate, HE: AE=2:1, R_{f}=0.11) to afford ester. (0.030 g, 0.057 mmol, 91%) as a waxy solid.

¹H NMR (CDCl₃): 5.31 (m, 4H), 5.03-4.84 (m, 1H), 4.26-4.13 (m, 4H), 4.11-4.00 (m, 1.5H), 3.81 (m, 0.5H), 3.42 (s, 1.5H), 3.38 (s, 1.5H), 2.32 (t, J=6.0 Hz, 2H), 2.21-2.04 (m, 2H), 2.01 (m, 4H), 1.61 (m, 2H), 1.56-1.24 (m, 26H), 0.85 (t, J=6.8 Hz, 3H). ¹³C NMR (CDCl₃): 173.60 (s), 129.98 (s), 129.70 (s), 86.43 (dd, J=178.7, 171.0 Hz), 85.63 (dd, J=178.7, 171.0 Hz), 75.47 (d, J=8.4 Hz), 74.90 (d, J=12.6 Hz), 64.56 (d, J=3.6 Hz), 64.45 (d, J=5.4 Hz), 63.26 (dd, J=10.0, 5.6 Hz), 62.88 (t, J=6.9 Hz), 58.21(s), 57.50 (s), 34.15 (s), 33.81 (d, J=18.4 Hz), 31.88 (s), 29.74 (s), 29.67 (s), 29.49 (s), 29.29 (s), 29.15 (s), 29.08 (s), 27.19 (s), 27.14 (s), 24.88 (s), 22.66 (s), 16.43 (m), 14.08 (s). ¹⁹F NMR (CDCl₃): −207.30 (m), −212.72 (M). ³¹P NMR (CDCl₃): 19.25 (d, J=76.1 Hz), 18.71 (d, J=75.0 Hz). MS (CI) m/z 523.3 (M⁺+1, 100.00). HRMS, M⁺+1, Found: 523.3568. Calcd for C₂₇H₅₃FO₆P, 523.3564. [α]²⁰_{D}=−3.08 (c=0.26, EtOH).

Diethyl [1-fluoro-3 (S)-O-methyl-4-(linolenoyloxy)-butyl]Phosphonate 15b.

Method B: ¹H NMR (CDCl₃): 5.32 (m, 6H), 5.02-4.82 (m, 1H), 4.25-4.13 (m, 4H), 4.08 (dd, J=12.0, 4.4 Hz, 1H), 4.01 (dd, J=12.0, 4.8 Hz, 1H), 3.65-3.55 (m, 1H), 3.41 (s, 1.5H), 3.37 (s, 1.5H), 2.76 (t, J=8.0 Hz, 4H), 2.29 (t, J=8.0 Hz, 2H), 2.19-1.92 (m, 6H), 1.58 (m, 2H), 1.34-1.21 (m, 14H), 0.93 (t, J=7.6 Hz, 3H). ¹³C NMR (CDCl₃): 173.50 (s), 131.88 (s), 130.18 (s), 128.22 (s), 128.18 (s), 127.67 (s), 127.05 (s), 85.47 (dd, J=179.4, 171.8 Hz), 85.25 (dd, J=179.4, 171.8 Hz), 75.41 (d, J=12.3 Hz), 73.92 (d, J=11.5 Hz), 64.56 (s), 64.46 (s), 63.23 (dd, J=10.0, 6.9 Hz), 62.84 (t, J=6.9 Hz), 58.16 (s), 57.45 (s), 34.09 (s), 34.15 (s), 32.94 (d, J=21.1 Hz), 31.67 (d, J=21.1 Hz), 29.51(s), 29.10 (s), 29.02 (s), 27.13 (s), 25.55 (s), 25.46(s), 24.83 (s), 20.48 (s), 16.40 (m), 14.20 (s). ¹⁹F NMR (CDCl₃): −207.38 (m), −212.72 (m). ³¹P NMR (CDCl₃): 19.25 (d, J=75.0 Hz), 18.70 (d, J=75.0 Hz). MS (CI) m/z 519.4 (M⁺+1, 84.26),225.2 (M⁺-C₁₇H₂₉CO₂H—CH₃, 100.00). HRMS, M⁺+1, Found: 519.3254. Calcd for C₂₇H₄₉FO₆P, 519.3251.

Diethyl [1-fluoro-3 (S)-O-Methyl-4-(palmitoylozy)-butyl]Phosphonate 15c.

Method A: 88% yield. Method B: 83% yield. ¹H NMR (CDCl₃): 5.04-4.76 (m, 1H), 4.26-4.14 (m, 4H), 4.11-4.00 (m, 1.5H), 3.81 (m, 0.5H), 3.42 (s, 1.5H), 3.38 (s, 1.5H), 2.30 (t, J=8.0 Hz, 2H), 2.20-2.01 (m, 2H), 1.60 (m, 2H), 1.34 (t, J=8.0 Hz, 6H), 1.31 (m, 26H), 0.85 (t, J=6.8 Hz, 3H). ¹³C NMR (CDCl₃): 173.61 (s), 86.43 (dd, J=178.7, 171.0 Hz), 85.63 (dd, J=178.7, 171.0 Hz), 75.47 (d, J=9.3 Hz), 74.90 (d, J=16.1 Hz), 64.59 (s), 64.50 (s), 63.32 (dd, J=10.0, 6.8 Hz), 62.88 (t, J=6.9 Hz), 58.20 (s), 57.50 (s), 34.17 (s), 34.15 (s), 32.97 (d, J=21.5 Hz), 31.90 (s), 29.66 (s), 29.62 (s), 29.44 (s), 29.33 (s), 29.24 (s), 29.11 (s), 24.89 (s), 22.64 (s), 16.43 (d, J=5.3 Hz), 14.09 (s). ¹⁹F NMR (CDCl₃): −207.39 (m), −212.73 (m). ³¹P NMR (CDCl₃): 19.26 (d, J=75.0 Hz), 18.71 (d, J=75.0 Hz). MS (CI) m/z 497.4 (M⁺+1, 100.00). HRMS, M⁺+1, Found: 497.3402. Calcd for C₂₅H₅₁FO₆P, 497.3407. [α]²⁰_{D}=3.33 (c=0.36, EtOH).

[1-Fluoro-3 (S)-O-methyl-4-(oleoyloxy)-butyl]Phosphonate 16a. 95% yield. ¹H NMR (CD₃OD): 5.33 (m, 2H), 4.92-4.77 (m, 1H), 4.34-4.02 (m, 2H), 3.72-3.61 (m, 1H), 3.44 (m, 1.5H), 3.39 (s, 1.5H), 2.34 (m, 2H), 2.16-2.09 (m, 2H), 2.03 (m, 4H), 1.61 (m, 2H), 1.32-1.29 (m, 22H), 0.89 (t, J=6.4 Hz, 3H). ¹³C NMR (CD₃OD): 175.18 (s), 130.89 (s), 130.80 (s), 86.43 (dd, J=178.7, 171.0 Hz), 85.63 (dd, J=178.7, 171.0 Hz), 77.17 (d, J=12.3 Hz), 75.78 (d, J=12.6 Hz), 65.88 (s), 65.73 (s), 58.38 (s), 57.75 (s), 34.96 (s), 34.95 (s), 34.08 (d, J=19.9 Hz), 33.06 (s), 32.82 (d, J=20.0 Hz), 30.84 (s), 30.79 (s), 30.61 (s), 30.45 (s), 30.35 (s), 30.27 (s), 30.17 (s), 28.13 (s), 26.03 (s), 23.74 (s), 14.45 (s). ¹⁹F NMR (CD₃OD): −207.35 (m), −212.19 (m). ³¹P NMR (CD₃OD): 17.41 (d, J=75.0 Hz), 16.87 (d, J=75.0 Hz). MS (CI) m/z 449.2 (M⁺1+1-H₂O, 100.00), 185.0 (M⁺-C₁₇H₃₃CO₂H, 72.11). HRMS, M⁺+1, Found: 449.2823. Calcd for C₂₃H₄₃FO₅P, 449.2832. [α]²⁰_{D}=−0.94 (c=0.32, MeOH).

[1-Fluoro-3 (S)-O-methyl-4-(linolenoyloxy)-butyl]Phosphonate 16b. ¹H NMR (CD₃OD): 5.40-5.26 (m, 6H), 4.94-4.76 (m, 1H), 4.27 (dd, J=36.0, 8.0 Hz, 1H), 4.08 (dd, J=32.0, 12.0 Hz, 1H), 3.65 (m, 1H), 3.44 (s, 1.5H), 3.39 (s, 1.5H), 2.80 (m, 4H), 2.13-1.99 (m, 2H), 2.14-1.99 (m, 6H), 1.61 (t, J=8.0 Hz, 3H), 1.33 (m, 8H), 0.97 (t, J=8.0 Hz, 3H). ¹³C NMR (CD₃OD): 173.10 (s), 130.73 (s), 129.07 (s), 127.21 (s), 127.19 (s), 126.85 (s), 126.23 (s), 86.43 (dd, J=178.7, 171.0 Hz), 85.63 (dd, J=178.7, 171.0 Hz), 75.14 (d, J=12.2 Hz), 73.73 (d, J=14.6 Hz), 63.87 (s), 63.72 (s), 56.39 (s), 55.75 (s), 32.95 (s), 32.93 (s), 32.06 (d, J=18.4 Hz), 30.80 (d, J=19.9 Hz), 28.67 (s), 28.25 (s), 28.18 (s), 28.14 (s), 26.15 (s), 24.52 (s), 24.41 (s), 24.01 (s), 19.49 (s), 12.67 (s). ¹⁹F NMR (CD₃OD): −207.34 (m), −212.21 (m). ³¹P NMR (CD₃OD): 17.39 (d, J=72.9 Hz), 17.03 (d, J=73.8 Hz). MS (CI) m/z 445.2 (M⁺-OH, 62.43), 185.0 (M⁺-C₁₇H₂₉CO₂H, 100.00). HRMS, M⁺+1, Found: 445.2507. Calcd for C₂₃H₃₉FO₅P, 445.2519.

[1-Fluoro-3 (S)-O-methyl-4-(palmitoyloxy)-butyl]Phosphonate 16c. 97% yield. ¹H NMR (CD₃OD): 4.95-4.78 (m, 1H), 4.34-4.30 (m, 1H), 4.24-4.14 (m, 1H), 3.72-3.61 (m, 1H), 3.44 (s, 1.5H), 3.39 (s, 1.5H), 2.34 (t, J=6.0 Hz, 2H), 2.13-1.99 (m, 2H), 1.60 (m, 2H), 1.33 (m, 26H), 0.89 (t, J=6.8 Hz, 3H). ¹³C NMR (CD₃OD): 175.20 (s), 86.43 (dd, J=178.7, 171.0 Hz), 85.63 (dd, J=178.7, 171.0 Hz), 77.17 (d, J=8.5 Hz), 75.76 (d, J=16.1 Hz), 65.85 (s), 65.69 (s), 58.37 (s), 57.74 (s), 34.98 (s), 34.56 (s), 34.08 (d, J=22.12 Hz), 33.08 (s), 32.82 (d, J=18.40 Hz), 30.78 (s), 30.77 (s), 30.71 (s), 30.60 (s), 30.48 (s), 30.40 (s), 30.18 (s), 26.04 (s), 23.74 (s), 12.48 (s). ¹⁹F NMR (CD₃OD): −207.42 (m), −212.27 (m). ³¹P NMR (CD₃OD): 17.36 (d, J=73.8 Hz), 17.01 (d, J=75.0 Hz). MS (CI) m/z 423.2 (M⁺-OH, 85.63),185.0 (M⁺-C₁₅H₃₁CO₂H, 100.00). HRMS, M⁺+1, Found: 423.2673. Calcd for C₂₁H₄₁FO₅P, 423.2676. [α]²⁰_{D}=−2.27 (c=0.22, MeOH).

V. Synthesis of Monofluorinated LPA Analogs

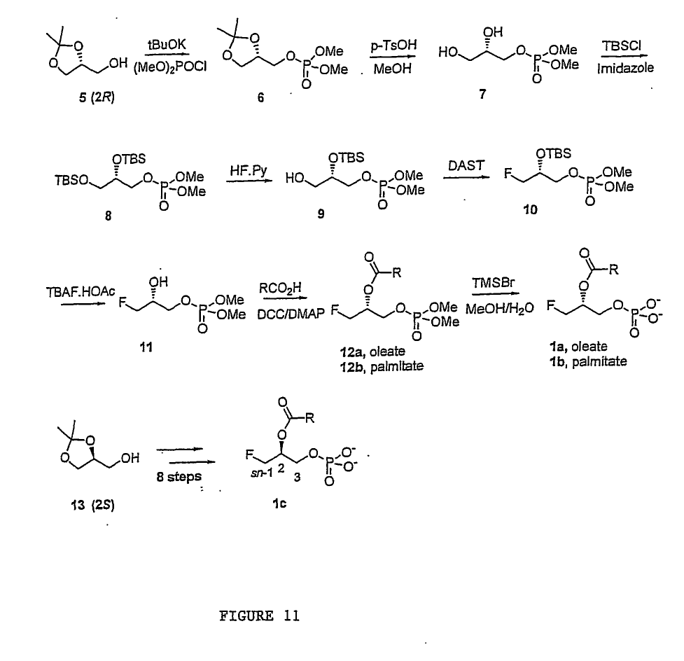

1-fluorodeoxy-(2R)-acyl-sn-glycerol-3-phosphates 1a and 1b were synthesized from commercially available (S)-isopropylideneglycerol 5 (FIG. 11). Alcohol 5 was first phosphorylated with dimethylphosphoryl chloride in the presence of t-BuOK to give dimethylphosphate 6 in 92% yield. Next, phosphate 6 was converted to 1-hydroxyl-2-(S)-(TBDMS)-3-phosphate in three steps. Acetonide hydrolysis with pTsOH/MeOH gave a crude diol, which was converted directly to the bis-silyl ether 8 by treatment with TBDMS-Cl and imidazole in anhydrous DMF. The more labile primary TBDMS was then cleaved selectively using pyridium-HF in pyridine-THF at rt Using an optimized selective deprotection, a 63% yield was obtained. Nucleophilic displacement of hydroxyl with DAST in anhydrous CH₂Cl₂ gave the corresponding monofluorinated compound 10, without affecting the 2-position TBDMS ether. The stable TBDMS ether was further deprotected with tetra-(n-butyl)ammonium fluoride (TBAF) in THF to give the secondary alcohol; neutralization of TBAF with acetic acid permitted this desilylation to occur without phosphate migration. DCC-promoted esterification of 11 with either oleic acid or palmitic acid provided good yields of esters 12a and 12b. Finally, treatment of each ester 12 with bromotrimethylsilane and subsequent addition of 5% aq. methanol provided the desired fluorinated LPA analogues 1a and 1b in nearly quantitative yield. Using the same procedure, the (2S-LPA analogue 1c was obtained from (R)-isopropylideneglycerol 13 in the analogous eight steps (5.6% overall yield) (FIG. 11).

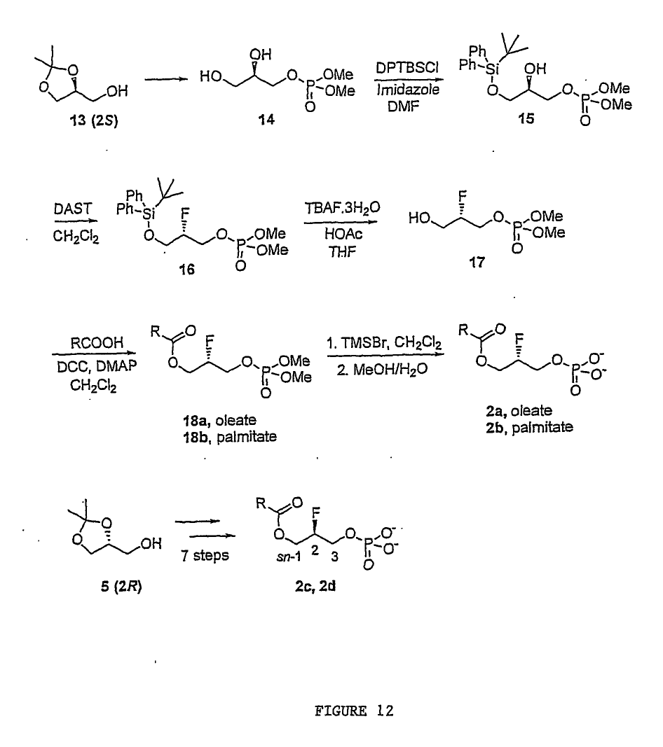

The 1-acyl-(2R)-fluorinedeoxy-sn-glycerol-3-phosphates 2 were synthesized from (R)-isopropylideneglycerol 13 (FIG. 12). As described above for diol 7, diol 14 was prepared by phosphorylation with dimethylphosphoryl chloride followed by acid hydrolysis. The primary alcohol was selectively protected as the TBDPS ether. Thus, treatment of diol 14 with the TBDPS chloride gave the sn-1 TBDPS ether 15. Deoxyfluorination of 15 gave good yields of the 2-fluorinated product 16. Deprotection of ether 16 with TBAF in THF gave alcohol 17, which was esterified with either oleic or palmitic acids as described above to give the target protected LPA derivatives 18a and 18b. Deprotection of the phosphotriester with bromotrimethylsilane afforded the desired fluorinated LPA analogues 2a and 2b. Similarly, the enantiomers 2c and 2d were synthesized from (S)-isopropylideneglycerol 5.

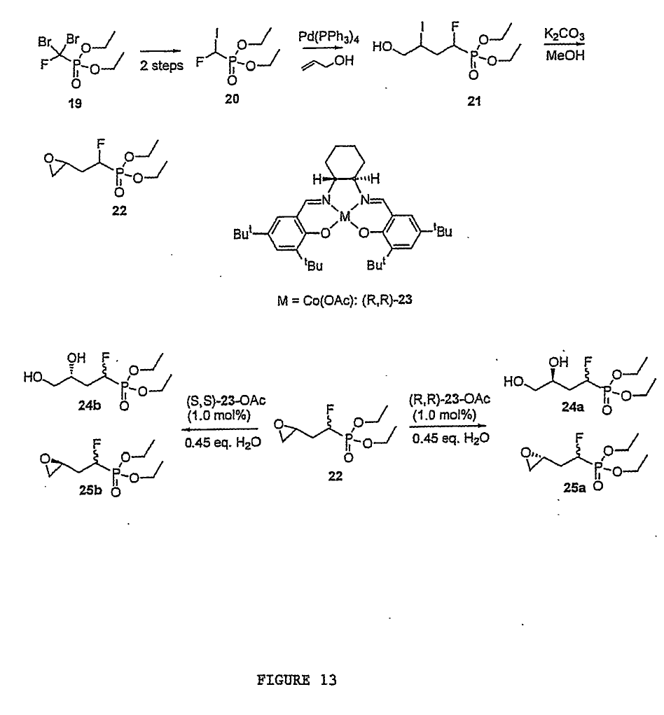

1-fluoro-3,4-epoxy-butylphosphonate 22 (IUPAC numbering) was prepared by addition of iodofluoromethylene-phosphonate 20 to allyl alcohol and subsequent base-induced cyclization of the iodohydrin 21 to epoxide 22 (FIG. 13). The HKR reaction, using two enantiomeric cobalt salen complexes 23 as catalysts, would be used for kinetic resolution of terminal epoxide of 22 to obtain enantiomerically-enriched diols 24a and 24b. These diols in turn would be mono-acylated to give the corresponding enantiomeric α-monofluoromethylene phosphonate LPA analogues 3.

FIG. 13 shows the final synthetic route for these analogues. First, iodomonofluoromethyl phosphonate 20 was prepared in good yield from commercially-available diethyl dibromofluoromethyl phosphonate 19 by tributylphosphine reduction and iodine quench of the intermediate zinc species. Next the tetrakis(triphenylphosphine)-palladium-catalyzed addition of phosphonate 20 to allyl alcohol in hexane gave the corresponding iodohydrin 21 in 79% yield. Treatment of the iodohydrin with dilute K₂CO₃/MeOH solution for 5 min at rt provided the desired epoxide 22 in good yield (72%). It is important to note that the racemic epoxide is also a mixture of fluorine epimers at C-1, as demonstrated by the two equal multiplets in the ¹⁹F-NMR spectra of this and subsequent intermediates. Next, reaction of racemic epoxide 22 with 0.45 eq of H₂O in a min volume of THF, in the presence of 1.0 mol % of (R,R)-23-OAc gave diol 24a in 90% ee and 73% isolated yield. Similarly, catalyst (S,S)-23-OAc provided the opposite configuration of diol 24b in 89% ee and 90% yield.

The epoxide and diol were readily separated by flash chromatography, providing a further extension of the scope of the HKR process, which was previously employed to make the difluoromethylene phosphonates. Each diol was isolated as an inseparable, equimolar mixture of two diastereomers epimeric at C-1. For initial assessment of biological activity, the separation of this epimeric mixture at the C-1 phosphonate methylene was not required.

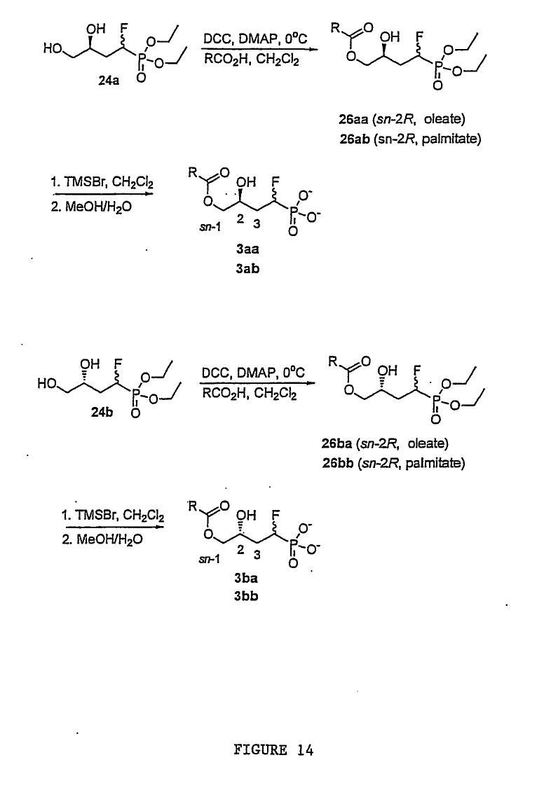

Regioselective acylation of the primary hydroxyl of diols 24 was readily accomplished (FIG. 14). Note that the numbering employed henceforth for the phosphonate LPA analogues 24, 25, 26, and 3 employs the sn-glycerol nomenclature for clarity of comparison with other LPA derivatives. Thus, treatment of 24a with 0.95 eq of oleic acid and 1.2 eq DCC and DMAP in CH₂Cl₂ at 0° C. gave 26aa in 42% yield after chromatography to remove a small amount of diester. The corresponding palmitate 26ab was similarly produced, as were the enantiomeric oleate 26ba and palmitate 26bb. Finally, LPA analogues 3 were obtained by dealkylation of the diethyl phosphonates 26 with excess bromotrimethylsilane (10.0 eq) for 8 h at rt.

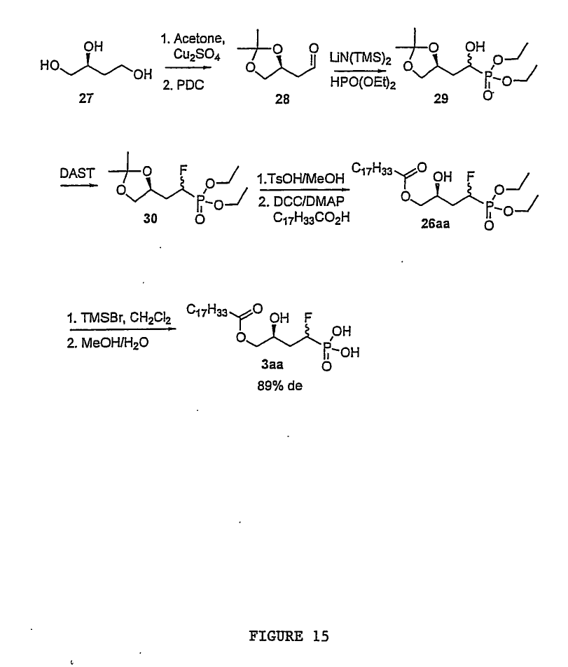

Since we were unable to separate the diastereomeric 1-fluoro-3-hydroxyl isomers of compounds 24, 26, or 3, we selected an alternative approach to prepare a diastereomerically enriched α-monofluorinated phosphonate. For this synthesis, (2S)-1,2,4-butanetriol 27 was chosen as the commercially-available chiral starting material. Protection as the isopropylidene acetal followed by oxidation with PDC gave aldehyde 28. The Pudovik reaction was then employed to introduce the C—P bond. Thus, the anion of diethyl phosphite was added to aldehyde 28 at −20° C. to give two chromatographically inseparable, α-hydroxyl phosphonates 29, in modest overall yield. This addition reaction occurred without diastereoselectivity, since two single sharp resonances at 25.37 and 24.47 ppm of equal intensity were observed in the ³¹P-NMR spectrum. This diastereomeric mixture was treated directly with DAST, which gave a pair of diastereomers in a 6.3:1 ratio as determined by both observed ³¹P NMR and ¹⁹F NMR in modest yield. After deprotection by acid hydrolysis and selective esterification, phosphonate 26aa was obtained in >89% de. Finally, TMSBr deprotection give the finally product 3aa showing >89% de (FIG. 15). As no reference materials are available, and NMR methods failed to define the relative geometries of the C—H bonds at C-1 and C-3, we cannot assign the absolute configuration at C-1 to this predominant stereoisomer.

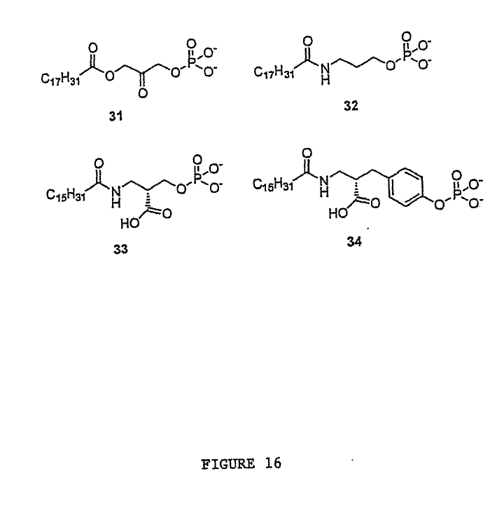

The preparation of receptor-specific agonists and antagonists for LPA receptors is an active area of ligand design. Structure-activity studies have demonstrated that analogues 31 and 32 (FIG. 16), lacking the 2-hydroxy group and structurally different analogues, such as the N-palmitoylserine and N-palmitoyltyrosine phosphoric acids 33 and 34 (FIG. 16), are potent competitive antagonists of LPA receptor function in Xenopus oocytes. However, thus far, a comprehensive analysis of fluorinated LPA analogues as selective agonists or antagonists for individual LPA receptors has not yet been reported. The monofluorinated analogues described herein provide a set of ligands to perform this comprehensive analysis.

Preliminary results indicate that compounds 1a, 1b and 2a-2d were all able to activate platelets. Moreover, compounds 1a and 1b were found to be partial agonists of the (18:1) LPA pain response and compound 1c was found to be somewhat more potent than natural 18:1 LPA on the LPA₃ receptor. However, analogues 1a, 1b and 2a-2d failed to show either significant agonist or antagonist activity when tested in insect cells expressing LPA₁, LPA₂, or LPA₃ receptors. Interestingly, monofluorinated sn-1 analogues 2a-2d were essentially equipotent with sn-1-oleoyl-LPA for the activation of the PPARγ nuclear receptor⁵. Thus, preliminary data demonstrate that particular fluorine substitutions can give selective agonists for LPA receptors, and that biological responses show both regioselectivity and enantioselectivity relative to the placement of the acyloxy and fluoro substituents. Most importantly, the α-monofluoromethylene-substituted LPA analogue 3aa was 1000-fold more potent than natural 18:1 LPA on the LPA₃ receptor. This response was also enantiospecific, clearly indicating that the α-fluorophosphonates are structurally informative and receptor-selective mimics for phosphate in LPA. The full biological data will be reported in due course.

Ligand recognition by GPCRs, as well as substrate recognition by enzymes, generally shows a strong preference for the naturally-occurring enantiomer. However, recognition of LPA by its receptors is an exception, as both the natural L(R) and unnatural D(S) stereoisomers of LPA have been reported to be equally active in selected bioassays. In contrast to the enantiomers of native LPA, preliminary data for fluorinated LPA analogues show that they are recognized in a stereoselective manner. For example, 1c (S) is approximately 100-fold more potent than 1a (R) on LPA₃ and 3aa (S) is similarly 100-fold more potent than 3ab (R). This distinction between LPA and the fluorinated derivatives raises the intriguing possibility that these analogues may interact with the ligand-binding pocket of LPA receptors in a manner different from LPA.

General Procedures. Except where noted, all reagents were purchased commercially. Solvents were of reagent grade and were distilled before use: THF was dried by distillation from sodium-benzophenone ketyl and methylene chloride was distilled from CaH₂. Reactions were performed under an inert atmosphere (N₂ or Ar) unless otherwise indicated. NMR spectra were recorded on 400 MHz (¹H), 101 MHz (¹³C), 162 MHz (³¹P) and 376 MHz (¹⁹F), at 25° C. Chemical shifts are reported relative to those of internal chloroform (δ_{H}=7.24), methanol (δ_{H}=4.78), or tetramethylsilane (δ_{H}=0.00) for ¹H; chloroform (δ_{C}=77.0) or methanol (δ_{C}=49.0) for ¹³C; CFCl₃ for ¹⁹F (δ_{F}=0.00); 85% H₃PO₄ (δ_{P}=0.00as external standard. Optical rotations were obtained at ambient temperature.

Dimethyl 1,2-(S)-isopropylidene-sn-glycerol-3-phosphate 6. t-BuOK (1.274 g, 11.35 mmol) was added to a stirred solution of (R)-isopropylideneglycerol (1.00 g, 7.57 mmol) and dimethyl chlorophosphate (1.367 g, 9.46 mmol) in CH₂Cl₂ (25 mL), stirred at rt for 1 h (complete by TLC). A saturated aq solution of NH₄Cl 40 mL was added, stirred 10 min, and the aq phase was extracted three times with CH₂Cl₂ (30 mL); the organic solution was dried (Na₂SO₄) and concentrated in vacuo. The crude product was purified on silica gel by elution with diethyl ether to give 1.62 g (6.75 mmol, 92% yield, Rf=0.30, diethyl ether) of pure product as a colorless oil. δ_{H}(CDCl₃): 4.22 (m, 1H), 3.95 (m, 4H), 3.69 (s, 3H), 3.66 (s, 3H), 1.33 (s, 3H), 1.24 (s, 3H). δ_{H}(CDCl₃): 106.69 (s), 73.88 (d, J=7.6 Hz), 67.36 (d, J=5.3 Hz), 65.84 (s), 54.23 (d, J=3.8 Hz), 26.51 (s), 25.06 (s). δ_{P}(CDCl₃): 2.23 (s). [α]²⁰_{D}=+2.28° (c=2.08, MeOH).

Dimethyl (2S)-1,2-di(tetra-butyldimethylsilyl)-sn-glycerol-3-phosphate 8. TsOH (54 mg, 0.283 mmol, 0.10 eq) was added to a solution of 6 (0.678 g, 2.825 mmol) in MeOH (10 mL), and the solution was stirred at rt for 24 h. After addition of NEt₃ (0.1 mL), the solvent was removed under reduced pressure. Following addition of anhydrous DMF (3 mL), imidazole (0.577 g, 8.475 mmol, 3.0 eq) and tert-butyldimethylsilyl chloride (TBDMSCl)(1.107 g, 7.345 mmol, 2.8 eq.), the reaction mixture was stirred at rt for an additional 36 h. The solution was diluted with water (15 mL) and ethyl acetate (20 mL), and the aqueous layer was separated and extracted three times with ethyl acetate (30 mL). The combined organic layers were dried (Na₂SO₄), concentrated in vacuo, and the residue was purified on silica gel (n-hexane/ethyl acetate 4:1, R_{f}=0.13) to afford 0.804 g (1.879 mmol, 67%) of a colorless liquid. δ_{H}(CDCl₃): 4.08 (m, 1H), 3.89 (m, 1H), 3.80 (m, 1H), 3.73 (d, J=1.2 Hz, 3H), 3.70 (d, J=1.2 Hz, 3H), 3.51 (d, J=5.2 Hz, 3H), 0.84 (s, 9H), 0.84 (s, 9H), 0.04 (s, 3H), 0.03 (s, 3H), 0.01 (s, 3H), 0.00 (s, 3H). δ_{C}(CDCl₃): 84.77 (d, J=6.1 Hz), 77.50 (d, J=7.6 Hz), 74.36 (d, J=6.2 Hz), 69.50 (s), 67.52 (d, J=4.5 Hz), 59.69 (d, J=6.3 Hz), 31.34 (s), 31.20 (s), 31.22 (s), 23.75 (s), 23.57 (s), 0.77 (s), 0.68 (s), 0.02 (s), 0.00 (s). δ_{P}(CDCl₃): 2.42 (s). MS (CI) m/z 429.1 (M⁺+1, 100.00). HRMS C₁₇H₄₂PSi₂O₆, Found: 429.2244; Calcd for 429.2230. [α]²⁰_{D}=+0.18° (c=2.25, MeOH).

Dimethyl (2S)-(tetra-butyldimethylsilyl)-sn-glycerol-3-phosphate 9. The HF•pyridine complex (70%, 0.31 mL) was added to a mixture of pyridine (1.40 mL) and a solution of the bis-TBDMS ether 8 (0.759 g, 1.773 mmol) in THF (10 mL). The reaction mixture was stirred for 24 h. After completion of the reaction (TLC), the solution was diluted with ethyl acetate (50 mL), washed with saturated NaCl solution (5 mL), and dried over anhydrous Na₂SO₄. After removal of the solvents, the residue was purified on silica gel (ethyl acetate, R_{f}=0.23) to afford a colorless liquid 0.254 g (0.814 mmol, 46%). δ_{H}(CDCl₃): 3.93 (m, 2H), 3.82 (m, 1H), 3.69 (d, J=1.2 Hz), 3.66 (d, J=1.2 Hz, 3H), 3.52 (dd, J=8.4, 4.4 Hz, 2H,), 0.79 (s, 9H), 0.01 (s, 3H), 0.00 (s, 3H). δ_{C}(CDCl₃): 76.06 (d, J=7.6 Hz), 72.40 (d, J=6.1 Hz), 67.93 (s), 59.29 (d, J=6.1 Hz), 30.57 (s), 22.91 (s), 0.11 (s), 0.00 (s). δ_{P}(CDCl₃): 2.788 (s). MS (CI) m/z 315.1 (M⁺+1, 100.00). HRMS C₁₁H₂₈SiPO₆, Found: 315.1412; Calcd for 315.1414. [α]²⁰_{D}=+0.28° (c=1.08, MeOH).

1-Phospho-2(S)-tetra-butyldimethylsilyl)-3-fluorine-propane-1,2-diol dimethyl ester 10. To a mixture of (0.035 g, 0.220 mmol) of DAST and 2 mL of dry CH₂Cl₂ at −78 C. was added dropwise a solution of (0.049 g, 0.157 mmol) alcohol in 1 mL of dry CH₂Cl₂. The mixture was stirred at −78 ° C. for 1 h, at rt for an additional 1 h. To the mixture was added 0.2 mL of methanol followed by neutralization with solid NaHCO₃. After concentration in vacuo, the residue was purified on silica gel (hexane-ethyl acetate, 1:1, R_{f}=0.25) to afford 0.026 g. (0.083 mmol, 53%) as a colorless oil. δ_{H}(CDCl₃): 4.35 (ddd, 1H), 4.24 (ddd, 1H), 4.02-3.86 (m, 3H), 3.69 (d, J=1.2 Hz, 3H), 3.66 (d, J=1.2 Hz, 3H), 0.79 (s, 9H), 0.05 (s, 6H). δ_{C}(CDCl₃): 88.46 (d, J=172.6 Hz), 74.76 (dd, J=20.7, 8.5 Hz), 72.26 (t, J=6.5 Hz), 59.31 (d, J=7.6 Hz), 30.55 (s), 22.98 (s), 0.00 (s). δ_{P}(CDCl₃): 2.252 (s). δ_{F}(CDCl₃): 230.50 (td, J=47.0, 20.7 Hz). MS (CI) m/z 317.1 (M⁺+1, 100.00). HRMS C₁₁H₂₇FSiPO₅, Found: 317.1344; Calcd for 317.1349. [α]²⁰_{D}=+0.23° (c=0.33, MeOH).

1-Phospho-2(S)-(oleoyl)-3-fluorine-propane-1,2-diol dimethyl ester 12a. A solution of 10 (18 mg, 0.058 mmol) in THF (2 mL) was treated consecutively with acetic acid (13 μL, 0.231 mmol) and tetrabutylammoniumfluoride trihydrate (73 mg, 0.231 mmol) at rt. After stirring for 18 h, the reaction was complete (TLC control), the solvent was evaporated under reduced pressure and the crude product was purified on a short column of silica gel to afford a colorless liquid. To the crude alcohol 11 and 42 mg, 47 μL, 0.147 mmol of oleic acid in dry CH₂Cl₂ (1 mL) at rt was added dropwise a solution of DCC (30 mg, 0.147 mmol) and DMAP (6 mg, 0.048 mmol) in dry CH₂Cl₂ (1 mL). The solution was stirred at rt for 18 h, filtered, concentrated in vacuo, and the residue was purified on silica gel (n-hexane-ethyl acetate 1:1, R_{f}=0.28) to afford 12 mg of a waxy solid (0.026 mmol, 45%). δ_{H}(CDCl₃): 5.28 (m, 2H), 5.14 (dm, J=20.8 Hz, 1H), 4.51 (dd, J=46.8, 4.0 Hz, 2H), 4.15 (m, 2H), 3.73 (d, J=2.4 Hz, 3H), 3.70 (d, J=2.4 Hz, 3H), 2.30 (t, J=7.2 Hz, 2H), 1.90 (m, 4H), 1.56 (m, 4H), 1.14 (m, 20H), 0.81 (t, J=6.4 Hz, 3H). δ_{C}(CDCl₃): 173.00 (s), 130.26 (s), 129.93 (s), 80.22 (d, J=172.0 Hz), 70.29 (d, J=28.6 Hz), 64.64 (t, J=6.5 Hz), 54.74 (s), 54.68 (s), 34.32 (s), 34.17 (s), 32.12 (s), 29.98 (s), 29.90 (s), 29.53 (s), 29.36 (s), 29.30 (s), 29.24 (s), 27.44 (s), 27.38 (s), 25.84 (s), 25.16 (s), 25.01 (s), 22.89 (s), 14.32 (s). δ_{P}(CDCl₃): 2.185 (s). δ_{F}(CDCl₃): −234.50 (td, J=47.0, 20.7 Hz). MS (CI) m/z 467.0, (M³⁰+1, 100.00), 341.2 (M⁺-OPO(OMe)², 56.20). HRMS C₂₃H₄₅FPO₆, Found: 467.2921; Calcd for 467.2904. [α]²⁰_{D}=+0.69° (c=0.36, MeOH).

1-Phospho-2(S)-(palmitoyl)-3-fluorine-propane-1,2diol Dimethyl Ester 12b. A solution of 10 (22 mg, 0.071 mmol) in THF (2 mL) was treated consecutively with acetic acid (16 μL, 0.282 mmol) and tetrabutylammoniumfluoride trihydrate (89 mg, 0.282 mmol) at rt. The crude alcohol 11 was directly esterified with palmitic acid (following the protocol above for 12a) and purified on silica gel (n-hexane-ethyl acetate 1:1, R_{f}=0.28) to afford 11 mg of a waxy solid (0.025 mmol, 35%). δ_{H}(CDCl₃): 5.20 (dm, J=21.0 Hz, 1H), 4.57 (dd, J=46.8, 4.0 Hz, 2H), 4.25 (m, 2H), 3.79 (d, J=2.8 Hz, 3H), 3.76 (d, J=2.4 Hz, 3H), 2.36 (t, J=9.6 Hz, 2H), 193 (m, 2H), 1.62 (m, 4H), 1.24 (m, 20H), 0.87 (t, J=9.6 Hz, 3H). δ_{C}(CDCl₃): 173.0 (s), 80.84 (d, J=173.4 Hz), 70.27 (d, J=7.64 Hz), 70.07 (d, J=7.4 Hz), 64.64 (t, J=6.7 Hz), 54.74 (s), 54.68 (s), 29.88-29.86 (m), 29.81 (s), 29.57 (s), 29.45 (s), 29.27 (s). δ_{P}(CDCl₃): 2.171 (s). δ_{F}(CDCl₃): −234.49 (td, J=47.0, 21.0 Hz). MS (CI) m/z 441.3 (M⁺+1, 20.84), 225, (M⁺—H₂O—C₁₂H₂₅, 100.00). HRMS C₂₁H₄₃FPO₆, Found: 441.2790; Calcd for 441.2781. [α]²⁰_{D}=+0.91° (c=0.29, MeOH).

1-Phospho-2(S)-(oleoyl)-3-fluorine-propane-1,2-diol 1a. Thoroughly dried ester 12a (8 mg, 0.017 mmol, 5 h under high vacuum) was dissolved in dry methylene chloride (1 mL) at rt, bromotrimethylsilane (9 μL, 0.052 mmol) was added via syringe and the reaction was stirred for 4 h. When TLC indicated that all of the reactant had been consumed, the solvent was removed under reduced pressure and the residue dried in vacuo. The residue was dissolved in 95% methanol (1 mL) for 1 h, the solvent was then removed under reduced pressure and the product dried in vacuo to give 6 mg of a colorless oil (CH₂Cl₂:CH₃OH:H₂O=20:10:1, R_{f}=0.39, 0.014 mmol, 82% yield.). δ_{H}(CD₃OD): 5.24 (m, 2H), 5.11 (dm, J=20.4 Hz, 1H,), 4.49 (dd, J=47.2, 4.8 Hz, 2H), 4.03 (m, 2H), 2.29 (t, J=7.6 Hz, 2H), 1.93 (m, 4H), 1.61-1.54 (m, 4H), 1.20 (m, 17H), 0.81 (t, J=6.4 Hz, 3H). δ_{C}(CD₃OD): 173.80 (s), 130.86 (s) 130.53 (s), 80.72 (d, J=171.9 Hz), 70.79 (d, J=28.4 Hz), 65.09 (t, J=6.5 Hz), 34.75 (s), 34.60 (s), 33.72 (s), 33.55 (s), 31.87 (s), 29.65 (s), 29.60 (s), 29.41 (s), 29.25 (s), 29.15 (s), 29.08 (s), 28.98 (s), 28.91 (s), 26.93 (s), 14.35 (s). δ_{P}(CD₃OD): 0.843 (s). δ_{F}(CD₃OD): −235.96 (td, J=47.0, 20.7 Hz). m/z 438.0 (M⁺, 0.30), 314.2, (M^{+—OPO(OH)}₂, 100.00), 157, (M^{+—OCOR,} 62.91). MS (CI) m/z 439.3 (M⁺+1, 45.34). HRMS, M⁺+1, Found: 439.2634. Calcd for C₂₁H₄₁FO₆P, 439.2625 [α]²⁰_{D}=+0.57° (c=0.12, MeOH).

1-Phospho-2(S)-(palmitoyl)-3-fluorine-propane-1,2-diol 1b. Deprotection of 12b (11 mg, 0.025 mmol, 5 h drying at 0.01 mg Hg) was conducted as described above for 12a to give 6 mg of phosphate 1b as a colorless oil (CH₂Cl₂:CH₃OH:H₂O=20:10:1, R_{f}=0.37, 0.019 mmol, 78% yield). δ_{H}(CD₃OD): 5.22 (dm, J=21.0 Hz, 1H), 4.58 (dd, J=47.2, 3.2 Hz, 2H), 4.25 (m, 2H), 2.36 (t, J=9.6 Hz, 2H), 1.93 (m, 2H), 1.76 (m, 2H), 1.62 (m, 4H), 1.29 (m, 18H), 0.87 (t, J=6.8 Hz, 3H). δ_{C}(CD₃OD): 173.40 (s), 81.24 (d, J=173.3 Hz), 70.67 (d, J=7.5 Hz), 70.47 (d, J=7.4 Hz), 64.95 (t, J=6.6 Hz), 32.78 (s), 32.14 (s), 29.93 (s), 29.88 (s), 29.71 (s), 29.59 (s), 25.26 (s), 25.00 (s), 24.63 (s), 22.91 (s), 14.32 (s). δ_{P}(CD₃OD): 1.742 (s). δ_{F}(CD₃OD): −234.63 (td, J=46.0, 21.0 Hz). MS (CI) m/z 413.3 (M⁺+1, 51.22). HRMS, M⁺+1, Found: 413.2479. Calcd for C₁₉H₃₉FO₆P, 413.2468 [α]²⁰_{D}=+0.81° (c=0.14, MeOH).

1-Phospho-2(S)-(oleoyl)-3-fluorine-propane-1,2-diol 1c. Colorless oil, δ_{H}(CD₃OD): 5.24 (m, 2H), 5.11 (dm, J=20.4 Hz, 1H,), 4.49 (dd, J=47.2, 4.8 Hz, 2H), 4.03 (m, 2H), 2.29 (t, J=7.6 Hz, 2H), 1.93 (m, 4H), 1.61-1.54 (m, 4H), 1.20 (m, 17H), 0.81 (t, J=6.4 Hz, 3H). δ_{C}(CD₃OD): 173.80 (s), 130.86 (s), 130.53 (s), 80.72 (d, J=171.9 Hz), 70.79 (d, J=28.4 Hz), 65.09 (t, J=6.5 Hz), 34.75 (s), 34.60 (s), 33.72 (s), 33.55 (s), 31.87 (s), 29.65 (s), 29.60 (s), 29.41 (s), 29.25 (s), 29.15 (s), 29.08 (s), 28.98 (s), 28.91 (s), 26.93 (s), 14.35 (s). δ_{P}(CD₃OD): 0.840 (s). δ_{F}(CD₃OD): −235.96 (td, J=46.6, 20.6 Hz). [α]²⁰_{D}=−0.71° (c=0.29, MeOH).

Dimethyl 1-(tetra-butyldiphenylsilyl)-2-(R)-sn-glycerol-3-phosphate 15. TsOH (0.594 g, 3.0 mmol, 0.15 eq) was added to a solution of (4.80 g, 20.00 mmol) in MeOH (100 mL), and the solution was stirred at rt for 24 h. Following addition of solid NaHCO₃, the mixture was filtered, concentrated in vacuo, and purified on silica gel (methanol-ethyl acetate 1:5, R_{f}=0.26) to afford 3.64 g (18.2 mmol, 91%) of diol 14 as a colorless liquid. To a solution of the crude diol 14 (3.45 g, 17.25 mmol) in anhydrous DMF (120 mL), was added imidazole (3.41 g, 50.03 mmol, 2.9 eq) and tert-butyldiphenylsilyl chloride (TBDBSCl) (6.16 g, 22.43 mmol, 1.3 eq). The reaction mixture was stirred at 0° C. for 8 h, then at rt for 12 h. The solution was diluted with ethyl acetate (100 mL), and the solution was washed with saturated NH₄Cl aq solution and brine. After drying with anhydrous Na₂SO₄, the organic layer was concentrated in vacuo and purified on silica gel (ethyl acetate, R_{f}=0.48) to afford 5.10 g of a colorless liquid (11.68 mmol, 68%). δ_{H}(CDCl₃): 7.65 (m, 4H), 7.36 (m, 6H), 4.16 (m, 2H), 3.93 (m, 1H), 3.71 (d, J=3.0 Hz, 3H), 3.68 (d, J=2.0 Hz, 3H), 1.04 (s, 9H). δ_{C}(CDCl₃): 135.20 (s), 135.18 (s), 132.74 (s), 132.73 (s), 129.51 (s), 127.47 (s), 70.20 (d, J=6.1 Hz), 68.52 (d, J=6.1 Hz), 63.61 (s), 54.05 (dd, J=6.1, 2.3 Hz), 26.49 (s), 18.88 (s). δ_{P}(CDCl₃): 2.869 (s). MS (CI) m/z 438.9 (M⁺+1, 20.62), 380.9 (M⁺—C₄H₉, 39.84), 360.9 (M⁺—C₆H₅, 100.00). HRMS, M⁺+1, Found: 439.1685. Calcd for C₂₁H₃₂O₆PSi, 439.1706. [α]²⁰_{D}=−0.77 (c=0.31, MeOH).

1-Phospho-2(S)-fluorine-3-(tetra-butyldiphenylsilyl)-propane-1,3-diol dimethyl ester 16. To a mixture of DAST (1.77 g, 10.96 mmol) and 50 mL of dry CH₂Cl₂ at −78° C. was added dropwise a solution of (4.00 g, 9.13 mmol) alcohol in 20 mL of dry CH₂Cl₂. The mixture was stirred at −78° C. for 1 h, followed by 1 h at rt. The mixture was poured into a stirred mixture of saturated NaHCO₃ and ice chips, the extracted with CH₂Cl₂. The extract was washed with H₂O, dried (Na₂SO₄), filtered, and evaporated under reduced pressure. The oil was purified on silica gel (hexane-ethyl acetate, 1:1, R_{f}=0.19) on silica gel to afford 1.53 g (3.47 mmol, 38%) of 16 as a colorless liquid. δ_{H}(CDCl₃): 7.64 (m, 4H), 7.42 (m, 6H), 4.71 (dm, J=47.6 Hz, 1H), 4.30 (dm, J=23.6 Hz, 2H), 3.83 (m, 2H), 3.76 (d, J=2.4 Hz, 3H), 3.68 (d, J=2.4 Hz, 3H), 1.04 (s, 9H). δ_{C}(CDCl₃): 135.55 (s), 135.49 (s), 132.79 (s), 132.67 (s), 129.90 (s), 127.81 (s), 127.79 (s), 91.17 (dd, J=177.2, 6.9 Hz), 66.33 (dd, J=23.7, 5.3 Hz), 62.27 (d, J=25.3 Hz), 54.40 (d, J=6.1 Hz), 26.68 (s), 19.19 (s). δ_{F}(CDCl₃): −196.16 (1F, m). δ_{P}(CDCl₃): 2.278 (s). MS (CI) m/z 383.0 (M⁺—C₄H₉, 29.86), 363.0 (M⁺—C₆H₅, 100.00). HRMS, M⁺—C₄H₉, Found: 383.0875. Calcd for C₁₇H₂₁FO₅PSi, 383.0880. [α]²⁰_{D}=−4.880° (c=0.42, MeOH).

1-Phospho-2(S)-fluorine-propane-1,3-diol Dimethyl Ester 17. A solution of 16 (860 mg, 1.972 mmol) in THF (50 mL) was treated consecutively with acetic acid (0.46 mL, 7.888 mmol) and tetrabutylammoniumfluoride trihydrate (2.489 g, 7.888 mmol) at rt. After stiring for 16 h, the reaction was complete (TLC), and the mixture was concentrated and passed through a silica column (ethyl acetate, R_{f}=0.20) to afford 0.342 g (1.693 mmol, 86%) of 17 as a colorless liquid. δ_{H}(CDCl₃): 4.67 (dm, J=48.0 Hz, 1H), 4.23 (ddd, J=22.4, 7.6, 4.4 Hz, 2H), 3.77 (dm, J=19.6 Hz, 2H), 3.75 (d, J=2.0 Hz, 3H), 3.72 (d, J=2.0 Hz, 3H), 3.48 (br, 1H). δ_{C}(CDCl₃): 91.32 (dd, J=174.8, 6.1 Hz), 66.02 (dd, J=23.7, 5.3 Hz), 60.53 (d, J=23.8 Hz), 54.54 (dd, J=6.1, 3.8 Hz). δ_{F}(CDCl₃): −197.66 (1F, m). δ_{P}(CDCl₃): 2.453 (s). MS (CI) m/z 203.1 (M⁺+1, 100.00). HRMS, M⁺+1, Found: 203.0476. Calcd for C₅H₁₂FO₅P, 203.0485.

1-Phospho-2(R)-fluorine-3-(oleoyl)-propane-1,3-diol Dimethyl Ester 18a. To a solution of crude alcohol 17 (73 mg, 0.361 mmol) with oleic acid (113 mg, 0.397 mmol) in dry CH₂Cl₂ (3 mL) at rt was added dropwise a solution of DCC (112 mg, 0.542 mmol) and DMAP (27 mg, 0.217 mmol) in dry CH₂Cl₂ (3 mL). The solution was stirred at rt for 16 h and filtered, the solvent removed, and the residue was purified on silica gel (n-hexane-ethyl acetate 1:2, R_{f}=0.30) to afford 162 mg (0.347 mmol, 96%) of 18a as a waxy solid. δ_{H}(CDCl₃): 5.28 (m, 2H), 4.80 (dm, J=47.6 Hz, 1H), 4.24 (m, 4H), 3.74 (s, 3H), 3.72 (s, 3H), 2.86 (t, J=7.2 Hz), 1.94 (m, 4H), 1.56 (m, 2H), 1.22 (m, 20H), 0.81 (t, J=8.0 Hz, 3H). δ_{C}(CDCl₃): 173.07 (s), 129.87 (s), 129.57 (s), 88.67 (dd, J=178.0, 7.6 Hz), 65.77 (dd, J=24.5, 5.3 Hz), 61.97 (d, J=23.7 Hz), 54.39 (d, J=6.1 Hz), 33.80 (s), 31.77 (s), 29.63 (s), 29.54 (s), 29.38 (s), 29.18 (s), 29.00 (s), 28.94 (s), 28.92 (s), 27.07 (s), 27.02 (s), 24.67 (s), 22.54 (s), 13.96 (s). δ_{F}(CDCl₃): −195.98 (1F, m). δ_{P}(CDCl₃): 2.151 (s). MS (CI) m/z 467.4 (M⁺+1, 100.00), 341.3 (M⁺—C₂H₆PO₄, 32.11). HRMS, M⁺+1, Found: 467.2891. Calcd for C₂₃H₄₅FO₆P, 467.2938. [α]²⁰_{D}=−1.92° (c=2.52, MeOH).

1-Phospho-2(R)-fluorine-3-(palmitoyl)-propane-1,3-diol Dimethyl Ester 18b. The same procedure was followed as for 18a to give 18b as a waxy solid (n-hexane-ethyl acetate 1:2, R_{f}=0.30; 139 mg, 0.316 mmol, 91%). δ_{H}(CD₃Cl): 4.77 (dm, J=48.0 Hz, 1H), 4.17 (m, 4H), 3.77 (s, 3H), 3.68 (s, 3H), 2.26 (t, J=7.6 Hz, 2H), 1.53 (m, 2H), 1.16 (m, 24H), 0.78 (t, J=6.4 Hz, 3H). δ_{C}(CD₃OD): 173.43 (s), 88.57 (dd, J=178.7, 7.6 Hz), 65.87 (dd, J=23.8, 5.4 Hz), 61.92 (d, J=23.8 Hz), 54.43 (d, J=6.1 Hz), 33.77 (s), 31.72 (s), 29.49 (s), 29.45 (s), 29.39 (s), 29.25 (s), 29.16 (s), 29.03 (s), 28.89 (s), 24.62 (s), 22.48 (s), 13.87 (s). δ_{F}(CD₃OD): −196.11 (1F, m). δ_{P}(CD₃OD): 1.977 (s). MS (CI) m/z 441.3 (M⁺+1, 100.00), 315.3 (M⁺—C₂H₆PO₄, 38.53). HRMS, M⁺+1, Found: 441.2770. Calcd for C₂₁H₄₃FO₆P, 441.2781. [α]²⁰_{D}=−1.25° (c=1.25, CHCl₃).

1-Phospho-2(S)-fluorine-3-oleoyl-propane-1,3-diol 2a. Following the same procedure used above for 1a afforded analogue 2a as a white solid in 86% yield. δ_{H}(CD₃OD/CDCl₃, 2/1): 5.32 (m, 2H), 4.82 (dm, J=48.0 Hz, 1H), 4.37 (m, 2H), 4.05 (ddd, J=48.0, 5.8, 5.2 Hz, 2H), 2.35 (t, J=7.6 Hz, 3H), 2.00 (m, 4H), 1.62 (m, 2H), 1.29 (m, 20H), 0.87 (t, J=6.4 Hz, 3H). δ_{C}(CD₃OD/CDCl₃, 2/1): 174.10 (s), 129.86 (s), 129.69 (s), 90.70 (dd, J=175.0, 7.6 Hz), 64.47 (dd, J=24.5, 5.4 Hz), 64.13 (d, J=22.2 Hz), 34.63 (s), 32.64 (s), 30.45 (s), 30.40 (s), 30.22 (s), 30.03 (s), 29.97 (s), 29.89 (s), 29.79 (s), 27.82 (s), 27.80 (s), 25.57 (s), 23.35 (s), 14.37 (s). δ_{F}(CD₃OD/CDCl₃, 2/1): −196.35 (1F, m). δ_{P}(CD₃OD/CDCl₃, 2/1): 2.145 (s). MS (CI) m/z 437.2 (M⁺+1-2Na⁺, 86.37). HRMS, M⁺+1-2Na⁺, Found: 437.2429. Calcd for C₂₁H₃₉FO₆P, 437.2390. [α]²⁰_{D}:=+0.57° (c=0.58, MeOH).

1-Phospho-2(S)-fluorine-3-palmitoyl-propane-1,3-diol 2b was obtained similarly as a white solid in 91% yield. δ_{H}(D₂O/CD₃OD): 4.81 (dm, J=48.8 Hz, 1H), 4.24 (dd, J=7.6, 6.4 Hz, 2H), 3.87 (dm, J=5.7 Hz, 2H), 2.27 (t, J=5.2 Hz, 2H), 1.49 (m, 2H), 1.16 (m, 24H), 0.76 (t, J=6.0 Hz, 3H). δ_{C}(D₂O/CD₃OD): 173.43 (s), 88.57 (dd, J=178.7, 7.6 Hz), 65.87 (dd, J=23.8, 5.4 Hz), 61.92 (d, J=23.8 Hz), 33.77 (s), 31.72 (s), 29.49 (s), 29.45 (s), 29.39 (s), 29.25 (s), 29.16 (s), 29.03 (s), 28.89 (s), 24.62 (s), 22.48 (s), 13.87 (s). δ_{F}(D₂O/CD₃OD): −194.87 (1F, m). δ_{P}(D₂O/CD₃OD): 4.325 (s). MS (CI) m/z 441.4 (M⁺+1-2Na⁺, 100.00). HRMS, M⁺⁺1, Found: 411.2307. Calcd for C₁₉H₄₃FO₆P, 411.2312. [α]²⁰_{D}=−5.00° (c=0.08, MeOH/H₂O, 1/1, v/v).

1-Phospho-2(R)-fluorine-3-oleoyl-propane-1,3-diol 2c was obtained similarly as a white solid. [α]²⁰_{D}:=−0.69° (c=0.45, MeOH).

1-Phospho-2(R)-fluorine-3-palmitoyl-propane-1,3-diol 2d was obtained similarly as a white solid. [α]²⁰_{D}=−4.51° (c=0.24, MeOH:H₂O=1:1, v/v).

Diethyl [1-fluoro-3,4-epoxy-butyl]phosphonate 22. K₂CO₃ (0.375 g, 2.712 mmol) was added to a solution of iodohydrin 21 (0.160 g, 0.452 mmol) in MeOH (20 mnL). The reaction mixture was stirred for 10 min at rt, diluted with water, and extracted with CH₂Cl₂. The organic phase was dried (Na₂SO₄), filtered, and concentrated in vacuo. The residue was purified by flash chromatography on silica gel to give 69 mg. (0.307 mmol, 68%, n-hexane-ethyl acetate=1:2, R_{f}=0.21) of epoxide 22 as a colorless liquid. δ_{H}(CDCl₃): 4.94-4.70 (m, 1H), 4.18-4.09 (m, 4H), 3.09 (m, 1H), 2.79 (t, J=4.8 Hz, 0.5H), 2.72 (t, J=4.4 Hz, 0.5H), 2.50 (m, 1H), 2.21-2.08 (m, 2H), 1.28 (m, 6H). δ_{C}(CDCl₃): 86.85 (dd, J=172.6, 148.0 Hz), 86.32 (dd, J=172.6, 148.0 Hz), 63.24 (dd, J=7.6, 3.8 Hz), 62.88 (dd, J=10.8, 6.1 Hz), 48.40 (dd, J=14.6, 3.8 Hz), 48.17 (dd, J=16.9, 3.8 Hz), 47.54 (s), 46.32 (s), 33.73 (dd, J=20.6, 1.5 Hz), 32.79 (dd, J=19.9, 1.5 Hz), 16.33 (d, J=3.0 Hz), 16.27 (d, J=3.1 Hz). δ_{F}(CDCl₃): −207.82 (0.5F, m), −211.22 (0.5F, m). δ_{P}(CDCl₃): 18.02 (0.5d, J=73.8 Hz), 17.97 (0.5d, J=75.0 Hz). MS (CI) m/z 227.1 (M⁺+1, 15.81), 203.1 (M⁺+1, 11.28). HRMS, M⁺+1, Found: 227.0836. Calcd for C₈H₁₇FO₄P, 227.0849.

Hydrolytic Kinetic Resolution of Epoxide 22. A 10-mL flask equipped with a stir bar was charged with (R,R)-23 (26.7 mg, 43 μmol, 0.01 eq). The catalyst was dissolved in 0.4 mL of PhMe and treated with AcOH (10 μL, 0.177 mmol). The solution was allowed to stir at rt open to air for 30 min; the color changed from orange-red to a dark brown. The solution was concentrated in vacuo to leave a crude brown solid. The resulting catalyst residue was dissolved in a solution of epoxide 22 (1.00 g, 4.425 mmol) and THF (150 μL) at rt, the reaction flask was cooled to 0° C., and H₂O (36 μL, 1.991 mmol, 0.45 eq) was added dropwise over 5 min. The reaction was allowed to warm to rt while stirring for 14 h. The reaction mixture was diluted with 20 mL of CH₂Cl₂ and the precipitate was removed by passage through Celite 351. Flash chromatography on silica gel afforded (R)-epoxide 25a (0.485 g, 2.146 mmol, 97%, R_{f}=0.32, CH₂Cl₂:CH₃OH=20:1) and (S)-diol 24a (0.394 g, 1.615 mmol, 73%, R_{f}=0.34, CH₂Cl₂:CH₃OH=10:1). The ee value of 24a was 91%, which is obtained by conversion to the known²⁵ isopropylidene-protected ketal. A comparison of the reported optical rotation values was then made.

Diethyl [1-Fluoro-3(S), 4-dihydroxybutyl]phosphonate 24a was obtained as described above as a colorless liquid. δ_{H}(CDCl₃): 5.13-4.88 (m, 1H), 4.21-4.05 (m, 4H), 3.97-3.85 (br, 2H), 3.61-3.41 (m, 3H), 2.12-1.94 (m, 2H), 1.31 (m, 6H). δ_{C}(CDCl₃): 86.16 (dd, J=171.0, 180.0 Hz), 85.54 (dd, J=171.0, 180.0 Hz), 68.34 (dd, J=9.3, 3.1 Hz), 67.23 (dd, J=14.2, 1.8 Hz), 66.59 (s), 65.88 (s), 63.65 (d, J=7.6 Hz), 63.44 (d, J=6.8 Hz), 63.19 (d, J=6.9 Hz), 63.12 (d, J=6.1 Hz), 33.87 (d, J=20.0 Hz), 33.68 (d, J=19.1 Hz), 16.34 (d, J=5.3 Hz), 16.29 (d, J=4.6 Hz). δ_{F}(CDCl₃): −207.48 (0.5F, m), −211.53 (0.5F, m). δ_{P}(CDCl₃): 19.91 (0.5P, d, J=75.0 Hz), 19.40 (0.5P, d, J=76.1 Hz). MS (CI) m/z 245.2 (M⁺+1, 100.00), 231.1 (M⁺+2-CH₃, 3.27). HRMS, M⁺+1, Found: 245.0965. Calcd for C₈H₁₉FO₅P, 245.0954. [α]²⁰_{D}=−18.77 (c=3.08, MeOH).

Diethyl [1-difluoro-3(R)-3,4-epoxy-butyl]phosphonate 25a. Recovered in resolved form as described above as a colorless liquid. δ_{C}(CDCl₃): 4.97-4.72 (m, 1H), 4.21-4.12 (m, 4H), 3.14-3.10 (m, 1H), 2.83 (t, J=4.0 Hz, 0.5H), 2.75 (t, J=4.0 Hz, 0.5H), 2.54 (m, 1H), 2.29-2.08 (m, 2H), 1.32 (m, 6H). δ_{C}(CDCl₃): 85.92 (dd, J=180.9, 172.5 Hz), 86.17 (dd, J=180.2, 172.6 Hz), 63.35 (d, J=3.1 Hz), 63.28 (d, J=3.1 Hz), 63.00 (d, J=4.6 Hz), 62.93 (d, J=4.6 Hz), 48.49 (dd, J=14.6, 3.8 Hz), 48.26 (dd, J=17.6, 3.8 Hz), 47.63 (s), 46.41 (s), 37.80 (d, J=19.8 Hz), 32.85 (d, J=19.9 Hz), 16.40 (d, J=12.4 Hz), 16.35 (d, J=12.0 Hz). δ_{F}(CDCl₃): −207.73 (0.5F, m), −211.17 (0.5F, m). δ_{P}(CDCl₃): 18.07 (d, J=73.8 Hz). [α]²⁰_{D}=+9.75 (c=3.54, MeOH).

To obtain the enantiomeric diol 24b, the enantiomeric catalyst was employed as follows. A 10-mL flask equipped with a stir bar was charged with (S,S)-23 (20.3 mg, 34 □mol, 0.01 eq). The catalyst was dissolved in 0.4 mL of PhMe and treated with AcOH (7 μL, 0.134 mmol). The solution was allowed to stir at rt open to air for 30 min; the color changed from orange-red to a dark brown. The solution was concentrated in vacuo to leave a crude brown solid. The resulting catalyst residue was dissolved in epoxide (0.758 g, 3.354 mmol) and THF (120 μL) at rt, the reaction flask was cooled to 0° C., and H₂O (27 μL, 1.509 mmol, 0.45 eq) was added dropwise over 5 min. The reaction was allowed to warm to rt, stirred for 14 h, concentrated, and purified on silica gel to give (S)-epoxide 25b (0.369 g, 1.631 mmol, 98%) and (S)-diol 24b (0.375 g, 1.537 mmol, 90%). The ee value of diol 24b was 89%, was obtained by conversion of 24b to the known²⁵ ketal and comparison of the reported optical rotations.

Diethyl [1-fluoro-3(R), 4-dihydroxybutyl]phosphonate 24b was obtained as above as a colorless liquid. δ_{H}(CDCl₃): 4.97-4.72 (m, 1H), 4.21-4.12 (m, 4H), 3.14-3.10 (m, 1H), 2.83 (t, J=4.0 Hz, 0.5H), 2.75 (t, J=4.0 Hz, 0.5H), 2.54 (m, 1H), 2.29-2.08 (m, 2H), 1.32 (m, 6H). δ_{C}(CDCl₃): 86.17 (dd, J=180.2, 172.6 Hz), 85.92 (dd, J=180.9, 172.5 Hz), 63.35 (d, J=3.1 Hz), 63.28 (d, J=3.1 Hz), 63.00 (d, J=4.6 Hz), 62.93 (d, J=4.6 Hz), 48.49 (dd, J=14.6, 3.8 Hz), 48.26 (dd, J=17.6, 3.8 Hz), 47.63 (s), 46.41 (s), 37.80 (d, J=19.8 Hz), 32.85 (d, J=19.9 Hz), 16.40 (d, J=12.4 Hz), 16.35 (d, J=12.0 Hz). δ_{F}(CDCl₃): −207.73 (0.5F, m), −211.17 (0.5F, m). δ_{P}(CDCl₃): 19.91 (0.5P, d, J=75.0 Hz), 19.40 (0.5P, d, J=76.1 Hz). [α]²⁰_{D}=+16.30 (c=4.50, MeOH).

Diethyl [1-difluoro-3(R)-3,4-epoxy-butyl]phosphonate 25b was recovered in resolved form as a colorless liquid. δ_{H}(CDCl₃): 4.97-4.72 (m, 1H), 4.21-4.12 (m, 4H), 3.14-3.10 (m, 1H), 2.83 (t, J=4.0 Hz, 0.5H), 2.75 (t, J=4.0 Hz, 0.5H), 2.54 (m, 1H), 2.29-2.08 (m, 2H), 1.32 (m, 6H). δ_{C}(CDCl₃): 85.92 (dd, J=180.9, 172.5 Hz), 86.17 (dd, J=180.2, 172.6 Hz), 63.35 (d, J=3.1 Hz), 63.28 (d, J=3.1 Hz), 63.00 (d, J=4.6 Hz), 62.93 (d, J=4.6 Hz), 48.49 (dd, J=14.6, 3.8 Hz), 48.26 (dd, J=17.6, 3.8 Hz), 47.63 (s), 46.41 (s), 37.80 (d, J=19.8 Hz), 32.85 (d, J=19.9 Hz), 16.40 (d, J=12.4 Hz), 16.35 (d, J=12.0 Hz). δ_{F}(CDCl₃): −207.73 (0.5F, m), −211.17 (0.5F, m). δ_{P}(CDCl₃): 18.07 (d, J=73.8 Hz). [α]²⁰_{D}=+12.06 (c=2.33, MeOH).

Diethyl [1-fluoro-3(S)-hydroxyl-4-(oleoyloxy)butyl]phosphonate 26aa. To a solution of diol 24a (107 mg, 0.438 mmol) and oleic acid (118 mg, 0.416 mmol) in dry CH₂Cl₂ (2 mL) was added a solution of DCC (109 mg, 0.526 mmol) and DMAP (32 mg, 0.263 mmol) in dry CH₂Cl₂ (1 mL) at 0° C. The solution was stirred for 16 h at 0° C., filtered, concentrated in vacuo, and the residue was purified on silica gel (n-hexane-ethyl acetate, HE:AE=1:1, R_{f}=0.29) to afford ester 121 mg. (0.238 mmol, 51%) as a waxy solid. δ_{H}(CDCl₃): 5.29 (m, 2H), 5.10-4.89 (m, 1H), 4.22-3.98 (m, 7H), 3.48 (br, 1H), 2.29 (t, J=7.6 Hz, 2H), 2.18-2.03 (m, 2H), 1.93 (m, 4H), 1.58(m, 2H), 1.33-1.22 (m, 28H), 0.83 (t, J=7.2 Hz, 3H). δ_{C}(CDCl₃): 173.84 (s), 173.81 (s), 129.92 (s), 129.64 (s), 86.49 (dd, J=171.0, 172.6 Hz), 84.71 (dd, J=171.1, 172.6 Hz), 68.06 (s), 67.48 (s), 66.01 (dd, J=10.0, 3.8 Hz), 65.07 (dd, J=13.1, 3.0 Hz), 63.55 (d, J=6.9 Hz), 63.30 (d, J=6.9 Hz), 63.06 (d, J=6.9 Hz), 62.98 (d, J=8.4 Hz), 34.36 (d, J=19.9 Hz), 33.81 (d, J=18.4 Hz), 31.82 (s), 29.67 (s), 29.61 (s), 29.43 (s), 29.23 (s), 29.09 (s), 27.13 (s), 27.08 (s), 24.86 (s), 22.59 (s), 16.35 (m), 14.02 (s). δ_{F}(CDCl₃): −208.26 (0.5F, m), −211.75 (0.5F, m). δ_{P}(CDCl₃): 19.36 (0.5P, d, J=73.8 Hz), 19.10 (0.5P, d, J=76.1 Hz). MS (CI) m/z 509.4 (M⁺+1, 29.75), 463.3 (M⁺—OC₂H₅, 100.00). HRMS, M⁺+1, Found: 509.3400. Calcd for C₂₆H₅₁FO₆P, 509.3407. [α]²⁰_{D}=−2.61 (c=2.38, MeOH).

Diethyl [1-fluoro-3(S)-hydroxyl4-(palmitoyloxy)butyl]phosphonate 26ab was obtained similarly as a white solid, 51% yield. δ_{H}(CDCl₃): 5.11-4.90 (m, 1H), 4.23-3.99 (m, 7H), 3.42 (br, 1H), 2.31 (t, J=7.6 Hz, 2H), 2.19-1.90 (m, 2H), 1.68-1.55 (m, 2H), 1.33 (t, J=6.8 Hz, 6H), 1.60 (m, 24H), 0.84 (t, J=7.2 Hz, 3H). δ_{C}(CDCl₃): 173.92 (s), 173.89 (s), 86.56 (dd, J=171.0, 168.2 Hz), 84.78 (dd, J=171.0, 168.2 Hz), 68.10 (s), 67.53 (s), 66.11 (dd, J=9.3, 3.8 Hz), 65.21 (dd, J=13.0, 3.1 Hz), 63.48 (dd, J=24.6, 6.9 Hz), 63.05 (dd, J=9.3, 6.8 Hz), 49.03 (s), 34.36 (d, J=19.9 Hz), 31.87 (s), 29.63 (s), 29.60 (s), 29.41 (s), 29.22 (s), 29.09 (s), 25.59 (s), 24.86 (s), 22.63 (s), 16.41 (d, J=5.3 Hz), 16.37 (d, J=4.6 Hz), 14.06 (s). δ_{F}(CDCl₃): −208.37 (0.5F, m), −211.62 (0.5F, m). δ_{P}(CDCl₃): 19.34 (0.5P, d, J=73.8 Hz), 19.11 (0.5P, d, J=76.1 Hz). MS (CI) m/z 483.4 (M⁺+1, 55.29), 437.4 (M⁺—OC₂H₅, 100.00). HRMS, M⁺+1, Found: 483.3244. Calcd for C₂₄H₄₉FO₆P, 483.3251. [α]²⁰_{D}=−2.20 (c=1.00, MeOH).

[1-Fluoro-3(S)-hydroxyl-4-(oleoyloxy)butyl]phosphonate 3aa. Thoroughly dried precursor 26aa (117 mg, 0.203 mmol, 5 h under high vacuum) was dissolved in dry methylene chloride (1 mL) at room temperature, and bromotrimethylsilane (353 mg, 2.030 mmol) was added with a dry syringe and the mixture was stirred for 4 h. When TLC indicated that all of the reactant had been consumed, the solvents were removed in vacuo. The residue was dissolved in 95% methanol (1 mL) for 1 h and reconcentrated in vacuo to give final product 88 mg (0.195 mmol, 96% yield) of phosphonate 3aa. δ_{H}(CD₃OD): 5.34 (m, 2H), 5.21-5.17 (m, 1H), 4.79 (m, 1H), 3,68 (dd, J=11.60, 4.40 Hz, 1H), 3.57 (m, 1H), 2.35 (m, 4H), 2.01 (m, 4H), 1.63 (m, 2H), 1.33-1.22 (m, 20H), 0.89 (t, J=7.2 Hz, 3H). δ_{C}(CDCl₃): 174.33 (s), 174.17 (s), 130.84 (s), 130.74 (s), 88.16 (dd, J=170.3, 168.7 Hz), 86.39 (dd, J=170.3, 168.7 Hz), 71.30 (dd, J=14.6, 2.3 Hz), 69.52 (dd, J=14.6, 2.3 Hz), 35.12 (d, J=19.3 Hz), 34.93 (d, J=18.9 Hz), 33.04 (s), 30.84 (s), 30.77 (s), 30.61 (s), 30.44 (s), 30.35 (s), 30.26 (s), 30.16 (s), 30.13 (s), 28.14 (s), 28.13 (s), 23.72 (s), 14.55 (s). δ_{F}(CDCl₃): −208.60 (0.5F, m), −210.99 (0.5F, m). δ_{P}(CDCl₃): 16.21 (0.5P, d, J=72.7 Hz), 15.95 (0.5P, d, J=73.8 Hz). MS (CI) m/z 435.3 (M⁺—OH, 60.85), 283.3 (M⁺—C₄H₉—CFH₃PO₃, 100.00). HRMS, M⁺—OH, Found: 435.2678. Calcd for C₂₂H₄₁FO₅P, 435.2676. [α]²⁰_{D}=−2.13 (c=0.14, MeOH).

[1-Fluoro-3(S)-hydroxyl-4-(palmitoyloxy)butyl]phosphonate 3ab was obtained similarly from precursor 26ab in 91% yield. δ_{H}(CD₃OD): 5.27-5.18 (m, 1H), 4.78 (m, 1H), 3,68 (dd, J=10.80, 4.00 Hz, 1H), 3.57 (m, 1H), 2.40-2.25 (m, 4H, 1.64 (m, 2H), 1.33-1.22 (m, 24H), 0.89 (t, J=7.2 Hz, 3H). δ_{C}(CDCl₃): 172.33 (s), 172.30 (s), 87.06 (dd, J=170.3, 168.7 Hz), 85.29 (dd, J=170.3, 168.7 Hz), 69.33 (dd, J=14.2, 2.4 Hz), 67.56 (dd, J=14.2, 2.4 Hz), 33.04 (d, J=7.7 Hz), 31.92 (s), 31.06 (s), 28.77 (s), 28.75 (s), 28.71 (s), 28.58 (s), 28.47 (s), 28.39 (s), 28.15 (s), 24.05 (s), 23.97 (s), 23.92 (s), 21.72 (s), 12.48 (s). δ_{F}(CDCl₃): −208.73 (0.5F, m), −211.07 (0.5F, m). δ_{P}(CDCl₃): 16.21 (0.5P, d, J=72.7 Hz), 15.95 (0.5P, d, J=73.8 Hz). MS (CI) m/z 409.2 (M⁺+1-OH—CH₃, 2.29), 225.2 (M⁺—C₁₄H₂₉—OH, 100.00). HRMS, M⁺—OH—CH₃, Found: 408.2432. Calcd for C₂₀H₃₈FO₅P, 408.2441. [α]²⁰_{D}=−1.83 (c=0.17, MeOH).

Diethyl [1-fluoro-3(R)-hydroxyl-4-(oleoyloxy)butyl]phosphonate 26ba was obtained as a waxy solid in 56% yield. δ_{H}(CDCl₃): 5.29 (m, 2H), 5.10-4.90 (m, 1H), 4.22-3.98 (m, 7H), 3.44 (br, 1H), 2.30 (t, J=7.6 Hz, 2H), 2.18-2.03 (m, 2H), 1.93 (m, 4H), 1.56 (m, 2H), 1.33-1.22 (m, 28H), 0.83 (t, J=7.2 Hz, 3H). δ_{C}(CDCl₃): 173.84 (s), 173.81 (s), 129.92 (s), 129.64 (s), 86.49 (dd, J=171.0, 172.6 Hz), 84.71 (dd, J=171.1, 172.6 Hz), 68.06 (s), 67.48 (s), 66.01 (dd, J=10.0, 3.8 Hz), 65.07 (dd, J=13.1, 3.0 Hz), 63.55 (d, J=7.0 Hz), 63.30 (d, J=7.0 Hz), 63.06 (d, J=7.0 Hz), 62.98 (d, J=8.4 Hz), 34.36 (d, J=19.9 Hz), 33.81 (d, J=18.4 Hz), 31.82 (s), 29.67 (s), 29.61 (s), 29.43 (s), 29.23 (s), 29.09 (s), 27.13 (s), 27.08 (s), 24.86 (s), 22.59 (s), 16.35 (m), 14.02 (s). δ_{F}(CDCl₃): −208.29 (0.5F, m), −211.75 (0.5F, m). δ_{P}(CDCl₃): 19.36 (0.5P, d, J=73.8 Hz), 19.10 (0.5P, d, J=76.1 Hz). [α]²⁰_{D}=+2.47 (c=1.86, MeOH).

Diethyl [1-fluoro-3(R)-hydroxyl-4-(palmitoyloxy)butyl]phosphonate 26bb was obtained as a white solid in 53% yield. δ_{H}(CDCl₃): 5.11-4.90 (m, 1H), 4.20-3.99 (m, 7H), 3.42 (br, 1H), 2.29 (t, J=7.6 Hz, 2H), 2.19-1.90 (m, 2H), 1.58 (t, J=6.8 Hz, 2H), 1.33 (t, J=6.8 Hz, 6H), 1.60 (m, 24H), 0.83 (t, J=7.2 Hz, 3H). δ_{C}(CDCl₃): 173.88 (s), 173.85 (s), 86.00 (dd, J=178.7, 171.1 Hz), 85.23 (dd, J=178.7, 171.1 Hz), 68.06 (s), 67.50 (s), 66.05 (dd, J=10.1, 4.6 Hz), 65.08 (dd, J=10.1, 4.6 Hz), 63.44 (dd, J=25.3, 7.6 Hz), 63.04 (dd, J=6.8, 6.8 Hz), 34.37 (d, J=19.9 Hz), 31.85 (s), 29.61 (s), 29.57 (s), 29.53 (s), 29.38 (s), 29.28 (s), 29.19 (s), 29.07 (s), 22.61 (s), 16.38 (d, J=5.3 Hz), 16.34 (d, J=4.6 Hz), 14.03 (s). δ_{F}(CDCl₃): −208.28 (0.5F, m), −211.75 (0.5F, m). δ_{P}(CDCl₃): 19.37 (0.5P, d, J=73.8 Hz), 19.10 (0.5P, d, J=76.1 Hz). [α]²⁰_{D}=+3.01 (c=0.84, MeOH).

[1-Fluoro-3(R)-hydroxyl-4-(oleoyloxy)butyl]phosphonate 3ba was obtained in 94% yield from precursor 26ba. δ_{H}(CD₃OD): 5.34 (m, 2H), 5.33-5.17 (m, 1H), 4.79 (m, 1H), 3,68 (dd, J=11.60, 4.40 Hz, 1H), 3.59 (m, 1H), 2.35 (m, 4H), 2.02 (m, 4H), 1.61 (m, 2H), 1.33-1.22 (m, 20H), 0.89 (t, J=7.2 Hz, 3H). δ_{C}(CDCl₃): 174.38 (s), 174.22 (s), 130.84 (s), 130.74 (s), 88.16 (dd, J=170.25, 168.74 Hz), 86.39 (dd, J=170.25, 168.74 Hz), 71.30 (dd, J=14.58, 2.31 Hz), 69.52 (dd, J=J=14.58, 2.31 Hz), 35.12 (d, J=19.32 Hz), 34.93 (d, J=18.89 Hz), 33.04 (s), 30.84 (s), 30.77 (s), 30.61 (s), 30.44 (s), 30.35 (s), 30.26 (s), 30.16 (s), 30.13 (s), 28.14 (s), 28.13 (s), 23.72 (s), 14.55 (s). δ_{F}(CDCl₃): −208.68 (0.5F, m), −210.99 (0.5F, m). δ_{P}(CDCl₃): 16.01 (0.5P, d, J=72.86 Hz), 15.93 (0.5P, d, J=74.00 Hz). [α]²⁰_{D}=+2.01 (c=0.22, MeOH).

[1-Fluoro-3(R)-hydroxyl-4-(palmitoyloxy)butyl]phosphonate 3bb was obtained in 88% yield from precursor 26bb. δ_{H}(CD₃OD): 5.27-5.18 (m, 1H), 4.78 (m, 1H), 3,68 (dd, J=10.80, 4.00 Hz, 1H), 3.57 (m, 1H), 2.40-2.25 (m, 4H), 1.64 (m, 2H), 1.33-1.22 (m, 24H), 0.89 (t, J=7.2 Hz, 3H). δ_{C}(CDCl₃): 172.33 (s), 172.30 (s), 87.06 (dd, J=170.25, 168.74 Hz), 85.29 (dd, J=170.25, 168.74 Hz), 69.33 (dd, J=14.21, 2.35 Hz), 67.56 (dd, J=14.21, 2.35 Hz), 33.04 (d, J=7.68 Hz), 31.92 (s), 31.06 (s), 28.77 (s), 28.75 (s), 28.71 (s), 28.58 (s), 28.47 (s), 28.39 (s), 28.15 (s), 24.05 (s), 23.97 (s), 23.92 (s), 21.72 (s), 12.48 (s). δ_{F}(CDCl₃): −208.73 (0.5F, m), −211.07 (0.5F, m). δ_{P}(CDCl₃): 16.19 (0.5P, d, J=72.70 Hz), 15.84 (0.5P, d, J=73.84 Hz). [α]²⁰_{D}=+2.56 (c=0.13, MeOH).

1-Diethylphosphonyl-3,4-O-isopropylidene-1(R,S),3(S),4-butanetriol 29. To a solution of diethyl phosphite (3.80 g, 24.07 mmol) in 8 mL of THF at −78° C., was added (24.07 mL) of 1.0M lithium bis(trimethylsilyl)amide in THF. The solution was allowed to r.t. and stirred for 45 min, and then cooled down to −20° C. Aldehyde 28 (3.3 g, 22.92 mmol) in 20 mL of THF was transferred into the solution at this temperature. The reaction mixture was allowed to warm to r.t. slowly and stirred fro overnight and then quenched by slow addition of acetic acid (24.1 mmol, 1.39 mL) in 10 mL of ether. It was filtered through Celite which was washed with ethyl acetate. The organic solvents were concentrated to give a colorless oil which was purified by flash chromatography to afford the phosphonate 29.

1-Diethylphosphonyl-1-fluorine-3,4-O-isopropylidene-1(R,S),3(S),4-butanetriol 30 was prepared by DAST fluorination using the procedure described for compound 16. δ_{H}(CDCl₃): 4.70-5.01 (m, 1H), 4.04-4.35 (m, 6H), 3.54-3.66 (m, 1H), 1.90-2.28 (m, 2H), 1.30-1.38 (m, 12H). δ_{P}(CDCl₃), 18.65 (d, J=73.84 Hz, integration, 91.42), 18.36 (d, J=76.10 Hz, integration, 8.58). δ_{F}(CDCl₃): −207.52 (0.085F, m), −212.52 (0.915F, m).

VI. Synthesis of Cyclic LPA Analogs

General procedures. Chemicals were obtained from Aldrich and Arcos Chemical Corporation and were used without prior purification. Solvents used were of reagent grade and were distilled before use: THF was distillated from sodium wire. Methylene chloride was distillated from CaH₂. Reactions were performed under an inert atmosphere (N₂ or Ar) unless otherwise indicated. ¹H and ¹³C spectra were recorded at 400 MHz (¹H), 101 MHz (¹³C), 162 MHz (³¹P) and 376 MHz (¹⁹F), temp. 25° C. Chemical shifts are given in ppm with TMS as internal standard (δ=0.00); ³¹P, 85% H₃PO₄ (δ=0.00); ¹⁹F, CFCl₃ (δ=0.00). FIGS. 17, 21 and 22 provide reaction schemes for producing the cyclic compounds described below. FIGS. 18-20 provide proposed reactions schemes for producing cyclic compounds described herein.

(E)-(3R)-Diethyl 1-Fluoro-3,4-O-cyclohexylidene-3,4-dihydroxybut-1-enylphosphonate 2. Treatment of tetraethyl fluoromethylenebisphosphonate (0.184 mg, 0.601 mmol in 5 mL dry hexane) with n-BuLi (0.601 mL, 1.0 M solution in hexane) at −78° C. under dry nitrogen gas followed by addition of (R)-1,4-dioxaspiro[4,5]decane-2-carbaldehyde (0.143 g, 0.841 mmol) with stiring at −78° C. gave a mixture which was brought to room temperature slowly. Filtration and evaporation under reduced temperature, followed by chromatography (ethyl acetate/hexane: 3/2) gave two isomers 2 (R_{f}=0.19, 0.178 g, 0.553 mmol, 92%). ¹H NMR (CDCl₃): 5.99 (dt, J=39.2, 7.6 Hz, 1H), 4.98 (m, 1H), 4.17-4.08 (m, 5H), 3.63 (dd, J=7.6, 6.4 Hz, 1H), 1.56 (m, 10H), 1.32 (m, 6H). ¹³C NMR (CDCl₃): 151.85 (dd, J=278.0, 233.2 Hz), 124.36 (dd, J=27.6, 3.0 Hz), 110.6 (s), 68.67 (dd, J=12.3, 6.9 Hz), 68.45 (m), 63.29 (dd, J=5.3, 3.0 Hz), 36.09 (s), 35.17 (s), 24.97 (s), 23.78 (s), 16.17 (d, J=6.1 Hz). ¹⁹F NMR (CDCl₃): −127.04 (dd, J=99.0, 39.1 Hz, 1F). ³¹P NMR (CDCl₃): 4.68 (d, J=98.9 Hz). MS (CI) m/z 323 (M⁺+1, 69.89), 99 (OC₆H₁₁⁺, 100.00). HRMS, M⁺, Found: 322.1354. Calcd for C₁₄H₂₄FO₅P, 322.1345. [α]²⁰_{D}=+51.68 (c=0.15, EtOH).

(3R)-Diethyl 1-fluoro-3,4-O-cyclohexylidene-3,4-dihydroxybutyl-1-phosphonate (3). A solution of 2 (0.128 g, 0.398 mmol) in absolute ethanol (8 mL) containing 10% Pd-C catalyst (10 mg) was stirred at ambient temperature under hydrogen (1 atm) until gas uptake ceased (18 h). Filtration and evaporation under reduced pressure gave compound 3 as a colourless liquid (0.126 g, 0.390 mmol, 98% yield). ¹H NMR (CDCl₃): 4.99-4.76 (m, 1H), 4.33-4.01 (m, 5H), 3.63-3.54 (m, 1H), 2.25-1.98 (m, 2H), 1.56 (m, 8H), 1.31 (m, 8H). ¹³C NMR (CDCl₃): 109.70 (s), 109.66 (s), 86.14 (dd, J=179.4, 171.8 Hz), 86.00 (dd, J=179.4, 171.8 Hz), 71.92 (dd, J=11.5, 3.0 Hz), 71.27 (dd, J=11.5, 3.0 Hz), 68.94 (s), 68.33 (s), 63.09 (dd, J=39.9, 6.9 Hz), 62.98 (dd, J=33.7, 4.6 Hz), 36.70 (s), 36.1417 (s), 35.06 (s), 34.81 (s), 33.99 (d, J=19.1 Hz), 16.40 (d, J=6.1 Hz). ¹⁹F NMR (CDCl₃): −207.52 (m), −212.53 (m). ³¹P NMR (CDCl₃): 18.76 (d, J=73.8 Hz), 18.47 (d, J=73.8 Hz). MS (CI) m/z 325 (M⁺+1, 100.00). HRMS, M⁺, Found: 324.1519. Calcd for C₁₄H₂₆FO₅P, 324.1502. [α]²⁰_{D}=−5.59 (c=0.34, EtOH).

(3R)-Diethyl 1-fluoro-3,4-dihydroxybutane-1-phosphonate (4). TsOH (7 mg, 0.035 mmol, 0.10 eq.) was added to a solution of 3 (0.114 g, 0.352 mmol) in MeOH (5 mL), and the solution was stirred at room temperature for 24 h. After addition of solid NaHCO₃ to neutralize the reaction mixture, the solvent was removed under reduced pressure. Chromatography afforded the homogenous product 4 (75 mg, 0.306 mmol, 87%). ¹H NMR (CDCl₃): 5.11-4.87 (m, 1H), 4.19-4.08 (m, 5H), 3.96 (br, 1H), 3.79 (br, 1H), 3.59 (m, 1H), 3.40 (m, 1H), 2.15-1.77 (m, 2H), 1.30 (t, J=6.8 Hz, 8H). ¹⁹F NMR (CDCl₃): −207.43 (m), −211.70 (m). ³¹P NMR (CDCl₃): 19.89 (d, J=74.0 Hz), 19.36 (d, J=75.9 Hz). [α]²⁰_{D}=−13.42 (c=0.73, EtOH).

1-Fluoro-3 (S),4-dihydroxylbutane-phosphonate (5). A thoroughly dried sample of 4 (46 mg, 0.189 mmol, 5 h under high vacuum) was dissolved in anhydrous methylene chloride (1 mL) at room temperature. Bromotrimethylsilane (0.25 mL, 1.890 mmol) was added with a dry syringe and stirred 4 h. TLC indicated that all of the reactant had disappeared, then the solvent was removed under reduced pressure and the residue was dried under vacuum. The residue was then dissolved in 95% methanol (1 mL) for 1 h, and the solvent was removed under reduced pressure and the product dried under vacuum, to give 33 mg (0.176 mmol, 93% yield) of diol 5. ¹H NMR (CD₃OD): 4.90 (m, 1H), 3.92-3.79 (m, 1H), 3.50 (m, 2H), 2.15 (m, 2H), 3.57 (m, 1H). ¹³C NMR (CD₃OD): 88.16 (dd, J=170.3, 168.7 Hz), 86.39 (dd, J=170.3, 168.7 Hz), 70.10 (dd, J=8.4, 2.3 Hz), 68.41 (dd, J=13.1, 2.3 Hz), 67.48 (s), 66.64 (s), 35.30 (m). ¹⁹F NMR (CD₃OD): −207.35 (1F, m), −212.58 (1F, m). ³¹P NMR (CD₃OD): 18.00 (d, J=75.0 Hz), 17.57 (d, J=76.1 Hz).

1-Fluoro-3(S)-hydroxyl-4-oxyoleoylbutane-1,2-cyclic phosphonate (7). 1.0 M dicyclohexylcarbodiimide in methylene chloride solution (1.4 eq., 0.216 mL, 0.216 mmol) was added dropwise to diol 5 (29 mg, 0.154 mmol) in 50 mL of anhydrous DMF solution. After 12 h, the cyclization reaction was complete and compound 6 was formed. Several drops of water were added to quench the reaction. After removing the solvent, the crude residue containing 6 was dissolved in anhydrous pyridine (2 mL). To the pyridine solution was added oleyl chloride (1.4 eq., 0.084 mL, 0.216 mmol) with vigorous stirring. After stirring for 12 h at room temperature, the solvent was removed and the crude CHF-ccLPA product was then purified on a Sephadex LH-20 column, eluting with CH₂Cl₂:CH₃OH=(7:3). Appropriate fractions were collected (R_{f}=0.39, CH₂Cl₂:CH₃OH:H₂O=65:25:4, 43 mg, 0.100 mmol, 65%). The product was dissolved in 1.0 M triethylammonium bicarbonate (TEAB) buffer (pH 8.0) to give a slightly cloudy solution, which was absorbed onto a sodium ion-exchange column (Dowex 50WX8-200 resin, neutral Na⁺form). The desired mixed neutral sodium salt of 7 was eluted with Nanopure water. The product solution was lyophilized to give an amorphous white powder, which was stored in solid form at −80° C. under nitrogen atmosphere. ¹H NMR (CD₃OD /D₂O): 5.33 (m, 2H), 5.10-5.00 (m, 1H), 4.49-4.38 (m, 1H), 4.24-4.10 (m, 2H), 2.37 (m, 2H), 2.15 (m, 2H), 2.00 (m, 4H), 1.59 (m, 2H), 1.26 (m, 20H), 0.89 (t, J=7.2 Hz, 3H). ¹⁹F NMR (CD₃OD /D₂O): −197.97 (1F, m), −203.30 (1F, m). ³¹P NMR (CD₃OD/D₂O): 32.28 (d, J=65.0 Hz), 31.82 (d, J=67.4 Hz).

Dimethyl 4-(benzyloxy)-3-hydroxybutanephosphonate (9) (FIG. 21). A 2.5 M solution of n-BuLi (60 mL, 150 mmol) in hexane was added dropwise to a stirred solution of methylphosphonate (18.6 g, 16.25 mL, 150 mmol) in dry THF (150 mL) at −78° C. under a nitrogen atmosphere. After 15 min of stirring, a solution of the benzyl glycidol ether (8) (8.21 g, 7.65 mL, 50 mmol) in THF (25 mL) was added dropwise, followed by BF₃.OEt₂ (25.35 mL, 200 mmol), which was slowly introduced while maintaining the temperature below −70° C. After the solution was stirred for two more hours, the reaction was quenched with saturated NH₄Cl (150 mL) and was allowed to warm up to room temperature. The residue obtained after evaporation under reduced pressure was extracted with ethyl acetate (200 mL×4). The solution was washed with brine, dried with Na₂SO₄, and concentrated, and the residue was chromatographed (Acetone/hexane: 1/1, R_{f}=0.30) on silica gel to yield the pure hydroxy phosphonate ester. (14.8 g, 51.3 mmol, 100%). ¹H NMR (CDCl₃): 7.30-7.22 (m, 5H), 4.48 (s, 2H), 4.34 (m, 1H), 3.76 (d, J=10.8 Hz, 6H), 3.39 (m, 2H), 2.23 (m, 1H), 2.09 (m, 1H), 1.96 (m, 1H), 1.85 (m, 1H). ³¹P NMR (CDCl₃): 36.60 (s). MS (CI) m/z 289.1 (M⁺+1, 100.00). HRMS, M⁺+1, Found: 289.1211. Calcd for C₁₃H₂₂O₅P, 289.1217.

Methyl 3-hydroxyl-4benzylbutane-1,3-cyclic phosphonate (10). Dimethyl 4-(benzyloxy)-3-hydroxybutanephosphonate (16.0 g, 70.18 mmol) was dissolved in anhydrous toluene (450 mL) and PPTS (pyridinium p-toluene sulfonate, 34.0 g, 140 mmol) was added. The mixture is heated to 80° C. for 20 hours. After cooled to room temperature, H₂O (200 mL) was added, and the solution was extracted with ethyl acetate. The organic phase was dried with Na₂SO₄, and concentrated, and the residue was chromatographed (Acetone/hexane: 1/1, R_{f}=0.48) on silica gel to yield the pure hydroxy phosphonate ester. (7.97 g, 31.13 mmol, 44%). ¹H NMR (CDCl₃): 7.33-7.26 (m, 5H), 4.57 (s, 2H), 4.34 (m, 1H), 3.76 (d, J=10.8 Hz, 3H), 3.56 (m, 2H), 2.23 (m, 1H), 2.09 (m, 1H), 1.96 (m, 1H), 1.85 (m, 1H). ¹³C NMR (CDCl₃): 137.60 (s), 128.36 (s), 127.71 (s), 127.67 (s), 127.52 (s), 77.29 (d, J=9.96 Hz), 73.53 (s), 72.06 (d, J=6.13 Hz), 52.39 (d, J=6.93 Hz), 25.82 (s), 18.26 (d, J=121.17 Hz). ³¹P NMR (CDCl₃): 51.04 (s). MS (CI) m/z 257.1 (M⁺+1, 100.00). HRMS, M⁺, Found: 257.0980. Calcd for C₁₂H₁₈O₄P, 257.1017.

Methyl 3,4-dihydroxybutane-1,3-cyclic phosphonate (11). A solution of 10 (2.1 g, 8.203 mmol) in absolute methanol (100 mL) containing 10% Pd-C catalyst (0.83 g) was stirred at ambient temperature under hydrogen (1 atm) until gas uptake ceased (18 h). Filtration and evaporation under reduced pressure gave compound 11, which was purified on silica gel (1.06 g, 6.40 mmol, 78% yield). ¹H NMR (CDCl₃): 4.27 (m, 1H), 3.68-3.76 (m, 1H), 3.72 (d, J=12.0 Hz), 3.60 (m, 1H), 2.10-2.22 (m, 2H), 2.00 (m, 1H), 1.80 (m, 1H). ¹³C NMR (CDCl₃): 79.31 (d, J=10.0 Hz), 64.49 (d, J=6.1 Hz), 52.50 (d, J=6.9 Hz), 24.89 (s), 18.47 (d, J=120.65 Hz). ³¹P NMR (CDCl₃): 52.11 (s). MS (CI) m/z 167.0 (M⁺+1, 100.00). HRMS, M⁺, Found: 167.0474. Calcd for C₅H₁₂O₄P, 167.0475.

Methyl 3-hydroxyl-4-terta-Butyldimethylsilylbutane-1,3-cyclic phosphonate (12). Alcohol 11 (0.420 g, 2.53 mmol) was dissolved in anhydrous DMF (10 mL) and stirred with imidazole (0.206 g, 3.04 mmol, 1.2 equiv) and tert-butyldimethylsilyl chloride (TBSCl) (0.420 g, 2.78 mmol, 1.1 equiv) for 24 h at room temperature. The solution was diluted with water (5 mL) and ethyl acetate (20 mL), and the aqueous layer was separated and extracted with ethyl acetate (3×20 mL). The combined organic layers were dried with Na₂SO₄ and concentrated in vacuo, and the residue was purified on silica gel (hexanes-ethyl acetate 2:1, R_{f}=0.40) to afford TBDMS ether 12 as a colorless liquid 0.392 g (1.324 mmol, 67%). ¹H NMR (CD₃Cl): 4.22 (m, 1H), 3.76-3.71 (m, 5H), 2.24-2.06 (m, 2H), 1.97-1.74 (m, 2H), 0.84 (s, 9H), 0.02 (m, 6H). ¹³C NMR (CD₃Cl): 78.19 (d, J=9.2 Hz), 67.17 (d, J=6.1 Hz), 52.14 (d, J=6.9 Hz), 25.63 (s), 25.46 (d, J=23.0 Hz), 18.67 (d, J=122.68 Hz). 18.08 (s). −5.61 (s), −5.67 (s). MS (CI) m/z 281.2 (M⁺+1, 100.00). HRMS, M⁺+1, Found: 281.1342. Calcd for C₁₁H₂₆O₄PSi, 281.1346.

Methyl 3-hydroxyl-4-terta-Butyldimethylsilylbutane-1,3-cyclic thiophosphonate (13). A solution of 12 (0.553 g, 1.975 mmol) and Lawesson's Reagent (0.44 g, 1.086 mmol) in toluene (3 mL) was stirred and heated at reflux for 4 h. The reaction mixture was washed with water (3 mL) and extracted with toluene (3×3 mL). The combined extracts were dried over anhydrous Na₂SO₄, filtered, the solvent was removed in vacuum, and the residue was purified by flash column chromatography on silica gel (EtOAc/hexane, 1:10, R_{f}=0.30) to give 13 (0.392 g, 67% yield) as a colorless liquid. ¹H NMR (CD₃Cl): 4.37 (m, 1H), 3.67-3.71 (m, 5H), 2.12-2.32 (m, 4H), 0.85 (s, 9H), 0.05 (s, 6H). ¹³C NMR (CD₃Cl): 81.52 (d, J=3.9 Hz), 65.38 (d, J=6.8 Hz), 52.35 (d, J=6.9 Hz), 29.21 (d, J=84.46 Hz), 25.72 (s), −0.08 (s), −5.36 (s), −5.55 (s). ³¹P NMR (CD₃Cl): 113.43 (s). MS (CI) m/z 297.1 (M⁺+1, 100.00). HRMS, M³⁰ +1, Found: 297.1128. Calcd for C₁₁H₂₆O₃PSSi, 297.1146.

Methyl 3,4-dihydroxyl-butane-1,3-cyclic thiophosphonate (14). A solution of 13 (143 mg, 0.483 mmol) in THF (8 mL) was treated consecutively with acetic acid (83 μL, 1.449 mmol) and tetrabutylammoniumfluoride trihydrate (457 mg, 1.449 mmol) at room temperature. After the solution was stirred for 18 h the reaction was complete (TLC control), the solvent was then evaporated under reduced pressure and the crude product was purified on a short column of silica gel (acetone/hexane, 3:2, R_{f}=0.45) to afford a colorless liquid. (61 mg, 0.335 mmol, 69% yield.). ¹H NMR (CD₃Cl): 4.30 (m, 1H), 3.55-3.76 (m, 5H), 2.92 (m, 1H), 2.02-2.31 (m, 5H). ¹³C NMR (CD₃Cl): 82.21 (d, J=3.8 Hz), 64.49 (d, J=6.1 Hz), 52.52 (d, J=6.8 Hz), 29.53 (d, J=92.49 Hz), 25.39 (s). ³¹P NMR (CD₃Cl): 113.99 (s). MS (CI) m/z 183.0 (M⁺+1, 100.00). HRMS, M⁺+1, Found: 183.0245. Calcd for C₅H₁₂O₃PS, 183.0246.

Methyl 3-hydroxyl-4-oleylbutane-1,3-cyclic thiophosphonate (15). To a pyridine solution (3 mL) of 14 (47 mg, 0.258 mmol) was added oleyl chloride (1.4 eq., 0.14 mL, 0.362 mmol) with good stirring. After being stirred at room temperature for 12 h, the solvent was removed and the crude product was then purified on silica gel (Ethyl acetate/hexane, 1:2, R_{f}=0.40) to afford a colorless liquid (99 mg, 0.222 mmol, 86% yield.). ¹H NMR (CD₃Cl): 5.29 (m, 2H), 4.42 (m, 1H), 4.26 (m, 1H), 4.07 (m, 1H), 3.72 (d, J=12.0 Hz, 3H), 1.60-2.35 (m, 13H), 1.23-1.27 (m, 22H), 0.85 (t, J=6.9 Hz, 3H). ¹³C NMR (CD₃Cl): 173.50 (s), 129.99 (s), 129.70 (s), 78.72 (d, J=10.7 Hz), 65.42 (d, J=6.9 Hz), 52.61 (d, J=6.9 Hz), 34.00 (s), 33.36 (s), 31.87 (s), 29.73 (s), 29.66 (s), 29.49(s), 29.29 (s), 29.12 (s), 29.06 (s), 27.19 (s), 27.13 (s), 25.72 (s), 24.78 (s), 22.65 (s), 19.08 (s), 17.87 (s), 14.09 (s). ³¹P NMR (CD₃Cl): 112.96 (s). MS (CI) m/z 447.1 (M⁺+1, 100.00). HRMS, M⁺+1, Found: 447.2632. Calcd for C₂₃H₄₃O₄PS, 447.2648.

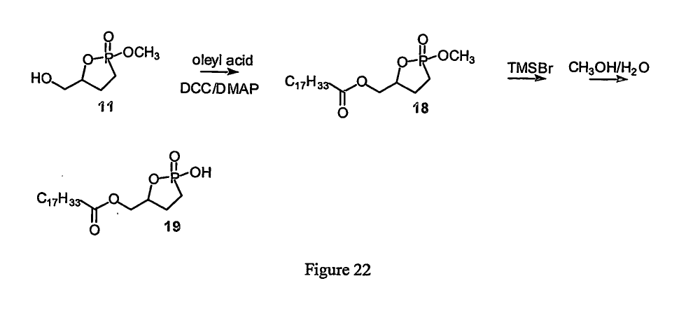

3-hydroxyl-4-oleylbutane-1,3-cyclic thiophosphonate (16). A solution of 15 (18 mg, 0.004 mmol) in 3 mL of tert-butylamine was refluxed for 48 h. Excess tert-butylamine was removed by evaporation and the resulting residue was purified on silica gel (CH₂Cl₂/CH₃OH/H₂O, 8:1:0.05, R_{f}=0.14) to afford a colorless liquid. (14 mg, 0.003 mmol, 75% yield.) The labile acid forms of these analogues were then converted to neutral sodium salts 17. Thus, product 16 was dissolved in 2 mL of 1.0 M triethylammonium bicarbonate (TEAB) buffer (pH 8.0) to give a slightly cloudy solution, which was absorbed to a sodium ion-exchange column (Dowex 50WX8-200 resin, neutral Na⁺ form). The desired mixed neutral sodium salt 17 was eluted with Nanopure water. The product solution was lyophilized to give sodium salt as white amorphous solid, which was stored in solid form at −80° C. under nitrogen atmosphere. The cyclic carbon PA analogue 19 (FIG. 22) was converted to the corresponding sodium salts in the same procedure. ¹H NMR of 16 (CD₃OD): 5.34 (m, 2H), 4.44 (m, 1H), 4.20 (dd, J=12.0, 3.2 Hz, 1H), 4.09 (dd, J=11.6, 6.0 Hz, 1H), 2.35 (t, J=8.0 Hz, 2H), 2.10-2.20 (m, 2H), 2.02 (m, 6H), 1.61 (m, 2H), 1.31 (m, 22H), 0.89 (t, J=6.9 Hz, 3H). ¹³C NMR (CD₃OD): 173.23 (s), 128.88 (s), 128.82 (s), 75.98 (s), 65.63 (s), 32.95 (s), 31.08 (s), 28.85 (s), 28.81 (s), 28.62 (s), 28.46(s), 28.34 (s), 28.30 (s), 28.21 (s), 26.13 (s), 23.97 (s), 21.75 (s), 12.47 (s). ³¹P NMR (CD₃OD): 93.88 (s). MS (CI) m/z 433.3 (M⁺+1, 100.00). HRMS, M⁺+1, Found: 433.2544. Calcd for C₂₂H₄₁O₄PS, 433.2547.

Methyl 3-hydroxyl-4-oleylbutane-1,3-cyclic phosphonate (18) (FIG. 22). To a solution of alcohol 11 (58 mg, 0.349 mmol) and oleic acid (108 mg, 0.419 mmol) in dry CH₂Cl₂ (2 mL) was added a solution of DCC (86 mg, 0.419 mmol) and DMAP (26 mg, 0.209 mmol) in dry CH₂Cl₂ (2 mL) at room temperature. The solution was stirred for 16 h at room temperature, filtered, concentrated in vacuo, and the residue was purified on silica gel (n-hexane-ethyl acetate, HE:AE=1:3, R_{f}=0.25 ) to afford ester 18 (87 mg, 0.202 mmol, 58%) as a liquid. ¹H NMR (CD₃Cl): 5.91 (m, 2H), 4.38 (m, 1H), 4.09 (ABd, J=12.0, 6.0 Hz, 1H), 4.26 (AB, J=12.0 Hz, 1H), 3.77 (d, J=12.0 Hz, 3H), 1.60-2.35 (m, 13H), 1.23-1.27 (m, 22H), 0.85 (t, J=6.9 Hz, 3H). ¹³C NMR (CD₃Cl): 173.46 (s), 129.99 (s), 129.70 (s), 75.67 (d, J=10.7 Hz), 65.56 (d, J=6.9 Hz), 52.54 (d, J=6.9 Hz), 34.00 (s), 33.36 (s), 31.87 (s), 29.73 (s), 29.66 (s), 29.49 (s), 29.29 (s), 29.12 (s), 29.06 (s), 27.19 (s), 27.13 (s), 25.72 (s), 24.78 (s), 22.65 (s), 18.48 (d, J=121.68 Hz), 14.09 (s). ³¹P NMR (CD₃Cl): 54.01 (s). MS (CI) m/z 431.4 (M⁺+1, 100.00). HRMS, M⁺+1, Found: 431.2929. Calcd for C₂₃H₄₃O₅P, 431.2931.

3-Hydroxyl-4-oleylbutane-1,3-cyclic phosphonate (19). Thoroughly dried precursor 18 (56 mg, 0.130 mmol, 5 h under high vacuum) was dissolved in dry methylene chloride (0.5 mL) at room temperature, and bromotrimethylsilane (70 mg, 0.456 mmol) was added with a dry syringe and the mixture was stirred for 1 h. When TLC indicated that all of the reactant had been consumed, the solvents were removed in vacuo. The residue was dissolved in 95% methanol (1 mL) for 1 h and reconcentrated in vacuo to give final product 51 mg (0.123 mmol, 94% yield) of phosphonate 19. ¹H NMR (CD₃OD): 5.34 (m, 2H), 4.50 (m, 1H), 4.27 (dd, J=12.0, 3.2 Hz, 1H), 4.10 (dd, J=11.6, 6.0 Hz, 1H), 2.35 (t, J=8.0 Hz, 2H), 2.10-1.80 (m, 8H), 1.62 (m, 2H), 1.31 (m, 22H), 0.89 (t, J=6.9 Hz, 3H). ¹³C NMR (CD₃OD): 173.67 (s), 129.73 (s), 129.63 (s), 76.53 (dd, J=12.0 Hz), 65.72 (dd, J=5.3 Hz), 33.67 (s), 31.94 (s), 29.73 (s), 29.67 (s), 29.44 (s), 29.34 (s), 29.22 (s), 29.17 (s), 29.07 (s), 29.05 (s), 27.01 (s), 25.08 (s), 24.79 (s), 22.61 (s), 19.29 (d, J=120.67 Hz), 13.38 (s). MS (CI) m/z 417.0 (M⁺+1, 40.31), 135.0 (M⁺—RCO₂, 100.00). HRMS, M⁺+1, Found: 417.2772. Calcd for C₂₂H₄₁O₅P, 417.2774.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

### REFERENCES

(1) Moolenaar, W. H. (1995) Lysophosphatidic acid, a multifinctional phospholipid messenger. J Biol Chem 270, 12949-52.
(2) Chun, J. (1999) Lysophospholipid receptors: implications for neural signaling. Crit Rev Neurobiol 13, 151-68.
(3) Sugiura, T., Nakane, S., Kishimoto, S., Waku, K., Yoshioka, Y., and Tokumura, A. (2002) Lysophosphatidic acid, a growth factor-like lipid, in the saliva. J Lipid Res 43, 2049-55.
(4) Sturm, A., and Dignass, A. U. (2002) Modulation of gastrointestinal wound repair and inflammation by phospholipids. Biochim Biophys Acta 1582, 282-8.
(5) Fang, X., Gaudette, D., Furui, T., Mao, M., Estrella, V., Eder, A., Pustihiik, T., Sasagawa, T., Lapushin, R., Yu, S., Jaffe, R. B., Wiener, J. R., Erickson, J. R., and Mills, G. B. (2000) Lysophospholipid growth factors in the initiation, progression, metastases, and management of ovarian cancer. Ann N Y Acad Sci 905, 188-208.
(6) Erickson, J. R., Hasegawa, Y., Fang, X., Eder, A., Mao, M., Furui, T., Aoki, J., Morris, A., and Mills, G. B. (2001) Lysophosphatidic acid and ovarian cancer: a paradigm for tumorogenesis and patient management. Prostaglandins Other Lipid Mediat 64, 63-81.
(7) Fang, X., Schummer, M., Mao, M., Yu, S., Tabassam, F. H., Swaby, R., Hasegawa, Y., Tanyi, J. L., LaPushin, R., Eder, A., Jaffe, R., Erickson, J., and Mills, G. B. (2002) Lysophosphatidic acid is a bioactive mediator in ovarian cancer. Biochim Biophys Acta 1582, 257-64.
(8) Contos, J. J., Ishii, I., and Chun, J. (2000) Lysophosphatidic acid receptors. Mol Pharmacol 58, 1188-96.
(9) Bandoh, K., Aoki, J., Taira, A., Tsujimoto, M., Arai, H., and Inoue, K. (2000) Lysophosphatidic acid (LPA) receptors of the EDG family are differentially activated by LPA species. Structure-activity relationship of cloned LPA receptors. FEBS Lett 478, 159-65.
(10) Yang, A. H., Ishii, I., and Chun, J. (2002) In vivo roles of lysophospholipid receptors revealed by gene targeting studies in mice. Biochim Biophys Acta 1582, 197-203.
(11) Murakami-Murofushi, K.; Shioda, M.; Kaji, K.; Yoshida, S.; Murofushi, H. J. Biol Chem. 1992, 267, 21512-7.
(12) Takahashi, Y.; Shimada, Y.; Shioda, M.; Yoshida, S.; Murofushi, H.; Murakami-Murofushi, K. Cell Struct. Funct. 1993, 18, 135-8.
(13) Murakami-Murofushi, K.; Kaji, K.; Kano, K.; Fukuda, M.; Shioda, M.; Murofushi, H. Cell Struct. Funct. 1993, 18, 363-70.
(14) Kobayashi, S.; Tokunoh, R.; Shibasaki, M.; Shinagawa, R.; Murakami-Murofushi, K. Tetrahedron Lett. 1993, 34, 4047-50.
(15) Murakami-Murofushi, K.; Kobayashi, S.; Oniinura, K.; Maysumoto, M.; Shioda, M.; Yoshida, S.; Shoji, M.; Murofushi, H. Biochim. Biophys. Acta. 1995, 1258, 57-60.
(16) Fischer, D. J.; Liliom, K.; Guo, H.; Nusser, N.; Virag, T.; Murakami-Murofushi, K.; Kobayashi, S.; Erickson, J. P.; Sun, G.; Miller, D. D.; Tigyi, G. Mol. Pharmacol. 1998, 54, 979-88.
(17) Mukai, M.; Imamura, F.; Ayaki, M.; Shinkai, K.; Iwasaki, T.; Murakami-Murofushi, K.; Murofushi, H.; Kobayashi, S.; Yamamoto, T.; Nakamura, H.; Akedo, H. Int. J. Cancer 1999, 81, 918-22.
(18) Mukai, M.; Kobayashi, S.; Murofushi, H.; Murofuishi, K. In PCT Int. 2002, p WO 0294286 (in Japanese).
(19) U.S. Pat. No. 6,495,532 B1
(20) Xu, Y.; Qian, L.; Prestwich, G. D. Organic Lett. 2003, 5, 2267-70.
(21) Nieschalk, J.; Batsanov, A. S.; O'Hagan, D.; Howard, J. A. K. Tetrahedron 1996, 52, 165-176.
(22) Wu, Y.; Zhou, C.; Robert, M. F. Biochemistry 1997, 36, 356-363.

1. A compound having the formula I 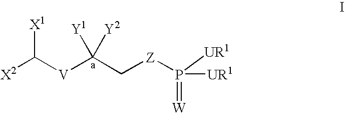
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting grouping;
R³ comprises a branched or straight chain C₁ to C₂₅ alkyl group, a cycloalkyl group, a heterocycloalkyl group, an aryl group, a heteroary group,
or the pharmaceutically acceptable salt or ester thereof,
wherein when Y¹ and Y² are different group, the sterochemistry at carbon a is either substantially R or substantially S, and
wherein the compound having the formula I is not 1-acyl-sn-glycerol 3-phosphate and 2-acyl-sn-glycerol 3-phosphate, and
wherein when V is not present, W is oxygen, X¹ and Y¹ are hydrogen, and X² is hydroxyl, then Y² is not hydroxyl.

2. The compound of claim 1, wherein each U and W comprises oxygen and V is not present.

3. The compound of claim 2, wherein Z comprises oxygen, X¹ comprises hydrogen, and X² comprises fluorine.

4. The compound of claim 3, wherein Y¹ comprises hydrogen, Y² comprises OC(O)R³, wherein R³ comprises a branched or straight chain C₁ to C₂₅ alkyl group, and R¹ comprises hydrogen.

5. (canceled)

6. The compound of claim 2, wherein Z comprises oxygen, Y¹ comprises hydrogen, and Y² comprises fluorine.

7. The compound of claim 6, wherein X¹ comprises hydrogen, X² comprises OC(O)R³, wherein R³ comprises a branched or straight chain C₁ to C₂₅ alkyl group, and each R¹ comprises hydrogen.

8. The compound of claim 2, wherein Z comprises CHF, Y¹ comprises hydrogen, and Y² comprises a hydroxyl group.

9. The compound of claim 8, wherein X¹ comprises hydrogen, X² comprises OC(O)R³, wherein R³ comprises a branched or straight chain C₁ to C₂₅ alkyl group, and each R¹ is hydrogen.

10. (canceled)

11. The compound of claim 8, wherein X¹ comprises hydrogen, X² is OC(O)R³, wherein R³ comprises a branched or straight chain C₁ to C₂₅ alkyl group, and each R¹ comprises ethyl.

12. (canceled)

13. The compound of claim 2, wherein Z comprises CHF, Y¹ comprises hydrogen, and Y² comprises an alkyl group.

14. The compound of claim 13, wherein X¹ comprises hydrogen, X² comprises a silyl group, a hydroxyl group, or OC(O)R³, wherein R³ comprises a branched or straight chain C₁ to C₂₅ alkyl group, and each R¹ comprises ethyl or each R¹ comprises hydrogen.

15. The compound of claim 2, wherein Z comprises CHF, Y¹ comprises hydrogen, and Y² comprises an OC(O)R³, wherein R³ comprises a branched or straight chain C₁ to C₂₅ alkyl group.

16. (canceled)

17. The compound of claim 2, wherein Z comprises CF₂.

18. The compound of claim 17, wherein Y¹ comprises hydrogen, Y² comprises OC(O)R³, wherein R³ comprises a branched or straight chain C₁ to C₂₅ alkyl group, and each R¹ comprises an ethyl group or a sodium ion.

19. The compound of claim 18, wherein X¹ comprises hydrogen and X² comprises OH or OC(O)R³, wherein R³ comprises a branched or straight chain C₁ to C₂₅ alkyl group.

20. The compound of claim 17, wherein X¹ comprises hydrogen, X² is OC(O)R³, wherein R³ comprises a branched or straight chain C₁ to C₂₅ alkyl group, and each R¹ comprises an ethyl group or a sodium ion.

21. The compound of claim 20, wherein Y¹ comprises hydrogen and Y² comprises OH or OC(O)R³, wherein R³ comprises a branched or straight chain C₁ to C₂₅ alkyl group.

22. The compound of claim 2, wherein Z comprises CH₂.

23. The compound of claim 22, wherein X¹ and X² comprise fluorine.

24. The compound of claim 23, wherein Y¹ comprises hydrogen, and Y² comprises a hydroxyl group, OR², or OC(O)R³.

25. The compound of claim 24, wherein each R¹ comprises hydrogen or a methyl group.

26. (canceled)

27. (canceled)

28. (canceled)

29. (canceled)

30. (canceled)

31. (canceled)

32. A compound having the formula VII 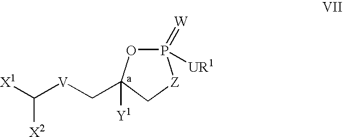
wherein
X¹, X², and Y¹ comprises, independently, hydrogen, fluorine, a hydroxyl group, a branched or straight chain C₁ to C₂₅ alkyl group, OR², OCH₂CH₂OR², OC(O)R³, or NC(O)R³;
U comprises oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises hydrogen, a branched or straight chain C₁ to C₂₅ alkyl group, or a cationic counterion;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group;
or the pharmaceutically acceptable salt or ester thereof,
wherein the stereochemistry at carbon a is either substantially R or substantially S,
wherein when W is oxygen, V is not present, X¹ and Y¹ are hydrogen, and X² is OC(O)R³, then Z is not CH₂ or oxygen.

33. The compound of claim 32, wherein Y¹ comprises hydrogen and Z comprises CHF, CF₂, or CH₂.

34. The compound of claim 33, wherein Z comprises CHF, each U comprises oxygen, and W comprises oxygen.

35. The compound of claim 34, wherein V is not present and R¹ comprises hydrogen or a branched or straight chain C₁ to C₂₅ alkyl group.

36. The compound of claim 35, wherein X¹ comprises hydrogen and X² comprises OH or OC(O)R³, wherein R³ comprises a branched or straight chain C₁ to C₂₅ alkyl group.

37. (canceled)

38. The compound of claim 32, wherein Z comprises CF₂, each U comprises oxygen, and W comprises oxygen.

39. The compound of claim 38, wherein V is not present and R¹ comprises hydrogen or a branched or straight chain C₁ to C₂₅ alkyl group.

40. The compound of claim 39, wherein X¹ comprises hydrogen and X² comprises OH or OC(O)R³, wherein R³ comprises a branched or straight chain C₁ to C₂₅ alkyl group.

41. (canceled)

42. The compound of claim 32, wherein Z comprises CHF or CF₂, each U comprises oxygen, and W comprises oxygen.

43. The compound of claim 42, wherein V comprises oxygen and R¹ comprises hydrogen or a branched or straight chain C₁ to C₂₅ alkyl group.

44. The compound of claim 43, wherein X¹ comprises hydrogen and X² comprises OH or OC(O)R³, wherein R³ comprises a branched or straight chain C₁ to C₂₅ alkyl group.

45. (canceled)

46. The compound of claim 32, wherein Z comprises CH₂, each U comprises oxygen, and W comprises oxygen.

47. The compound of claim 46, wherein V is not present and R¹ comprises hydrogen or a branched or straight chain C₁ to C₂₅ alkyl group.

48. The compound of claim 47, wherein X¹ comprises hydrogen and X² comprises OH or OC(O)R³, wherein R³ comprises a branched or straight chain C₁ to C₂₅ alkyl group.

49. (canceled)

50. The compound of claim 46, wherein V comprises oxygen and R¹ comprises hydrogen or a branched or straight chain C₁ to C₂₅ alkyl group.

51. The compound of claim 50, wherein X¹ comprises hydrogen and X² comprises a branched or straight chain C₁ to C₂₅ alkyl group.

52. (canceled)

53. The compound of claim 32, wherein Z comprises CH₂, each U comprises oxygen, and W comprises sulfur.

54. The compound of claim 53, wherein V is not present and R¹ comprises hydrogen or a branched or straight chain C₁ to C₂₅ alkyl group.

55. The compound of claim 54, wherein X¹ comprises hydrogen and X² comprises OH or OC(O)R³, wherein R³ comprises a branched or straight chain C₁ to C₂₅ alkyl group.

56. (canceled)

57. The compound of claim 32, wherein Z comprises sulfur, each U comprises oxygen, and W comprises oxygen.

58. The compound of claim 57, wherein V is not present and R¹ comprises hydrogen or a branched or straight chain C₁ to C₂₅ alkyl group.

59. The compound of claim 58, wherein X¹ comprises hydrogen and X² comprises OH or OC(O)R³, wherein R³ comprises a branched or straight chain C₁ to C₂₅ alkyl group.

60. (canceled)

61. The compound of claim 57, wherein V comprises oxygen and R¹ comprises hydrogen or a branched or straight chain C₁ to C₂₅ alkyl group.

62. The compound of claim 61, wherein X¹ comprises hydrogen and X² comprises OH or OC(O)R³, wherein R³ comprises a branched or straight chain C₁ to C₂₅ alkyl group.

63. (canceled)

64. A compound having the formula VII 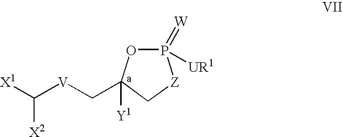
wherein
X¹, X², and Y¹ comprises, independently, hydrogen, fluorine, a hydroxyl group, a branched or straight chain C₁ to C₂₅ alkyl group, OR², OCH₂CH₂OR², OC(O)R³, or NC(O)R³;
U comprises oxygen, sulfur, or NR¹;
V is not present or when V is present, V comprises oxygen or sulfur;
W comprises oxygen or sulfur;
Z comprises sulfur, NR¹, CHF, CF₂, or CHOR²;
each R¹ comprises hydrogen, a branched or straight chain C₁ to C₂₅ alkyl group, or a cationic counterion;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group;
or the pharmaceutically acceptable salt or ester thereof,
wherein the stereochemistry at carbon a is either substantially R or substantially S.

65. The compound of claim 1, wherein the stereochemistry at carbon a is substantially R.

66. The compound of claim 1, wherein the stereochemistry at carbon a is substantially S.

67. A pharmaceutical composition comprising a pharmaceutically-acceptable compound and the compound of claim 1.

68. A method for preparing a compound having the formula III 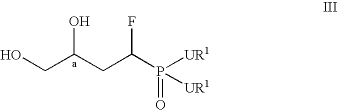
wherein each R¹ comprises, independently, hydrogen, a branched or straight chain C₁ to C₂₅ alkyl group, a cationic counterion, or both R¹ form a cyclic or heterocyclic group, and each U comprises, independently, oxygen, sulfur, or NR¹; and
the stereochemistry at carbon a is substantially R or substantially S,
or the pharmaceutically acceptable salt or ester thereof,
comprising
(a) reacting a compound having the formula IV 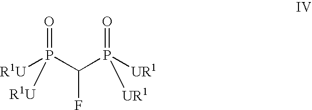
with a compound having the formula V 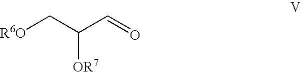
wherein R⁶ and R⁷ are protecting groups,
in the presence of a base;
(b) hydrogenating the compound produced in step (a); and
(c) deprotecting the compound produced in step (b) to produce a compound having the formula II.

69. (canceled)

70. (canceled)

71. A method for preparing the compound of claim 32, comprising reacting a compound having the formula VIII 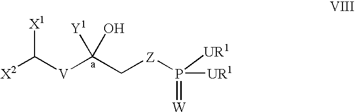
wherein
X¹, X², and Y¹ comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group;
or the pharmaceutically acceptable salt or ester thereof,
wherein the stereochemistry at carbon a is either substantially R or substantially S, with a dehydrating agent.

72. (canceled)

73. A method for improving wound healing in a subject in need of such improvement, comprising contacting the wound of a mammal with a compound having the formula I or VII or a pharmaceutical composition thereof 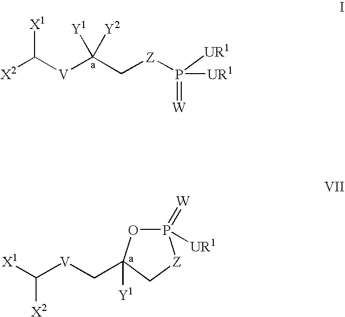
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

74. A method for treating or preventing in a subject a disease comprising administering to the subject a compound having the formula I or VII or a pharmaceutical composition thereof 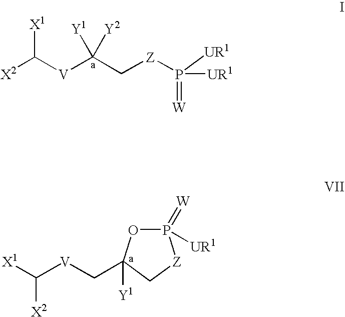
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl
group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S,
wherein the compound having the formula I is not 1-acyl-sn-glycerol 3-phosphate and 2-acyl-sn-glycerol 3-phosphate, and
wherein with formula VII, when W is oxygen, V is not present, X¹ and Y¹ are hydrogen, and X² is OC(O)R³, then Z is not CH₂ or oxygen.

75. The method of claim 74, wherein the disease comprises cancer or diabetes.

76. (canceled)

77. A method for reducing inflammation or an allergic response in a subject comprising administering to the subject a compound having the formula I or VII or a pharmaceutical composition thereof 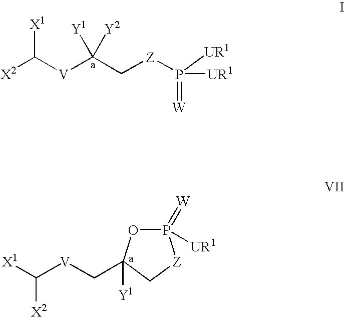
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

78. A method for increasing or altering cardiovascular function in a subject comprising administering to the subject a compound having the formula I or VII or a pharmaceutical composition thereof 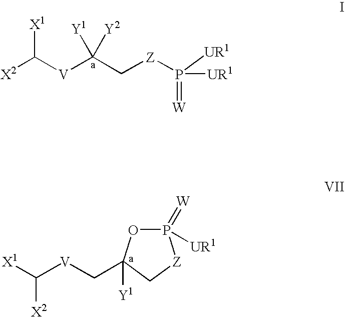
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

79. A method for maintaining or terminating embryonic development in a subject comprising administering to the subject a compound having the formula I or VII or a pharmaceutical composition thereof 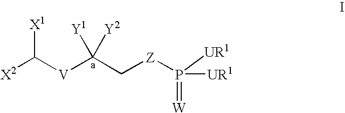 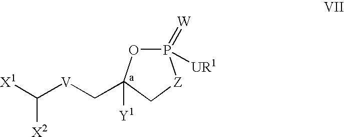
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

80. A method for eliciting or inhibiting platelet aggregation in a subject comprising administering to the subject a compound having the formula I or VII or a pharmaceutical composition thereof 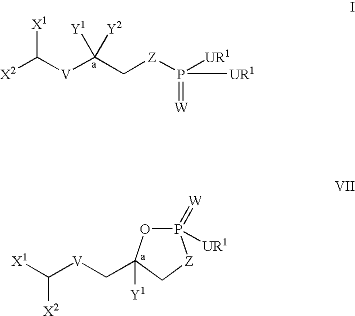
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R₁ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

81. A method for increasing or inhibiting cell growth and proliferation in a culture comprising contacting the cells in the culture with a compound having the formula I or VII or a pharmaceutical composition thereof 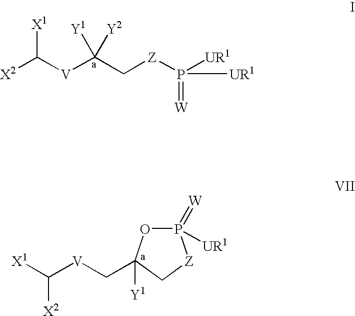
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR₂;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

82. A method of treating or preventing a disease in a subject comprising administering a compound having the formula I or VII or a pharmaceutical composition thereof as a PPARγ agonist 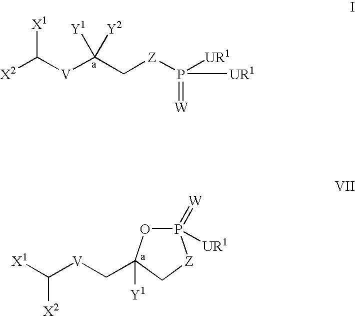
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

83. A method of treating or preventing a disease in a subject comprising administering a compound having the formula I or VII or a pharmaceutical composition thereof to inhibit a lipid phosphatase, lipid kinase, or phospholipase enzyme 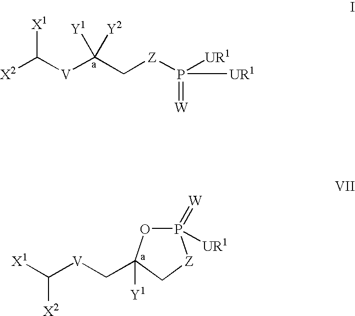
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

84. The use of a compound having the formula I or VII or a pharmaceutical composition thereof for targeting the discovery of a drug 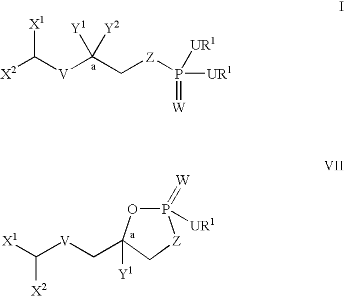
wherein
X¹, X², Y¹, and Y² comprises, independently, hydrogen, fluorine, a hydroxyl group, a branched or straight chain C₁ to C₂₅ alkyl group, OR₂, OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

85. A method for growing or proliferating cells in a culture comprising administering to the cells in the culture a compound having the formula I or VII or a pharmaceutical composition thereof 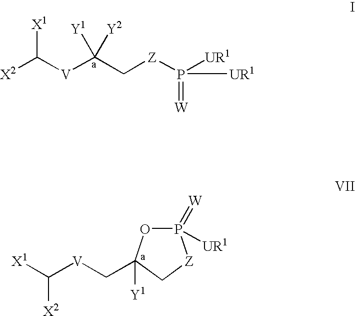
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group,
or the pharmaceutically acceptable salt or ester thereof,
wherein when Y¹ and Y² in formula I are different groups, the stereochemistry at carbon a is either substantially R or substantially S, and
wherein the compound having the formula I is not 1-acyl-sn-glycerol 3-phosphate and 2-acyl-sn-glycerol 3-phosphate.

86. A method for determining the activity of lysophosphatidic acid or phosphatidic acid, comprising the steps of:
a) measuring the activity of a compound having the formula I or VII 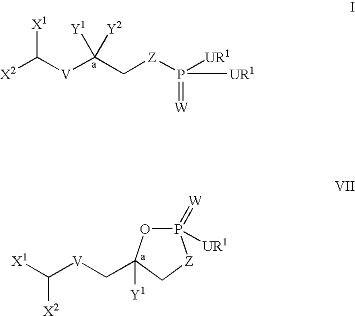
wherein
X¹, X², Y¹, and Y² comprises, independently, hydrogen, fluorine, a hydroxyl group, a branched or straight chain C₁ to C₂₅ alkyl group, OR², OCH₂CH₂OR², OC(O)R³, or NC(O)R³;
each U comprises, independently, oxygen, sulfur, or NR¹;
V is not present or when V is present, V comprises oxygen or sulfur;
W comprises oxygen or sulfur;
Z comprises oxygen, sulfur, NR¹, CH₂, CHF, CF₂, or CHOR²;
each R¹ comprises, independently, hydrogen, a branched or straight chain C₁ to C₂₅ alkyl group, a cationic counterion, or both R¹ form a cyclic or heterocyclic group;
R² comprises hydrogen, a branched or straight chain C₁ to C₂₅ alkyl group, a cycloalkyl group, a heterocycloalkyl group, an aryl group, a heteroaryl group or a protecting group;
R³ comprises a branched or straight chain C₁ to C₂₅ alkyl group, a cycloalkyl group, a heterocycloalkyl group, an aryl group, a heteroaryl group, or the pharmaceutically acceptable salt or ester thereof, wherein when Y¹ and Y² in formula I are different groups, the stereochemistry at carbon a is either substantially R or substantially S, and wherein the compound having the formula I is not 1-acyl-sn-glycerol 3-phosphate and 2-acyl-sn-glycerol 3-phosphate; and
b) measuring the same activity of lysophosphatidic acid or phosphatidic acid.

87. The method of claim 86, wherein the method comprises identifying agonists or antagonists of lysophosphatidic acid binding to or activating lysophosphatidic acid receptors of the edg class in a cell.

88. The method of claim 86, wherein the method comprises identifying agonists or antagonists of lysophosphatidic acid binding to or activating lysophosphatidic acid receptors of the non-edg class in a cell.


SEQUENCE LISTING (2 sequences)

SEQ ID NO: 1 - protein, 6 aa, Artificial Sequence
  Description of Artificial Sequence; note = synthetic construct
ZYRGDS

SEQ ID NO: 2 - protein, 4 aa, Artificial Sequence
  Description of Artificial Sequence; note = synthetic construct
GFLG